(19) United States
(12) Patent Application Publication
(10) Pub. No.: US 2024/0122952 A1
(43) Pub. Date: Apr. 18, 2024

(54) TOPICAL HUMAN MILK FORMULATIONS

(71) Applicant: PROLACTA BIOSCIENCE, INC., Duarte, CA (US)

(72) Inventors: Biranchi PATRA, Duarte, CA (US); Adam SUN, Duarte, CA (US); Victoria NIKLAS, Duarte, CA (US)

(21) Appl. No.: 18/262,344

(22) PCT Filed: Jan. 21, 2022
(86) PCT No.: PCT/US2022/013313

§ 371 Date: Jul. 20, 2023

Related U.S. Application Data
(60) Provisional application No. 63/195,392, filed on Jun. 1, 2021.
(60) Provisional application No. 63/140,544, filed on Jan. 22, 2021.

(51) Int. Cl. A61K 31/702; A61K 9/00; A61K 9/06; A61K 9/107; A61K 35/20; A61P 17/04; A61P 17/06

(52) U.S. Cl. CPC A61K 31/702; A61K 9/0014; A61K 9/06; A61K 9/107; A61K 35/20; A61P 17/04; A61P 17/06

(57) ABSTRACT

Provided herein are compositions, such as topical formulations, that contain one or more fractions, portions, or components of human milk and methods of using and manufacturing the same. In some aspects, the provided compositions include human milk cream or one or more fatty acids found in or native to human milk. Also included are methods for treating or preventing skin conditions, such as atopic dermatitis or psoriasis.

### CROSS-REFERENCE TO RELATED APPLICATIONS

This application claims priority from U.S. provisional application No. 63/140,544 filed Jan. 22, 2021, entitled “TOPICAL HUMAN MILK FORMULATIONS” and U.S. provisional application No. 63/195,392 filed Jun. 1, 2021, entitled “TOPICAL HUMAN MILK FORMULATIONS”; the contents of both are incorporated by reference in their entirety.

### FIELD OF THE INVENTION

Provided herein are compositions, such as topical formulations, that contain one or more fractions, portions, or components of human milk and methods of using and manufacturing the same. In some aspects, the provided compositions include human milk cream or one or more fatty acids found in or native to human milk. Also included are methods for treating or preventing skin conditions, such as atopic dermatitis or psoriasis.

### BACKGROUND OF THE INVENTION

The skin serves numerous functions, but its primary function is as a protective layer or barrier. An important role of the skin for terrestrial animals is to protect the water-rich internal organs from the dry external environment. In addition, the skin protects internal tissues from harmful chemical and physical forces as well as from the penetration of pathogens.

Existing creams, lotions, ointments, and other product formulations for skin care may, in some cases, improve skin appearance but fail to improve barrier function. In other instances, such treatments may be formulated to provide antioxidant, anti-inflammatory, or free-radical-scavenging effects but may fail to do so, e.g., due to an inability to penetrate or successfully integrate within the barrier. Other skin treatments, such as corticosteroids, may actually weaken barrier function. Others still, while purporting to be natural, are derived from potentially allergenic plant extracts or animal proteins. There exists a need for topical formulations that can treat and/or prevent skin conditions, promote wound healing, or decrease inflammation while avoiding unwanted secondary effects.

### SUMMARY OF THE INVENTION

Provided herein is a topical formulation comprising one or more human milk fractions, and one or more excipients, e.g., pharmaceutically acceptable excipients, wherein the one or more human milk factions comprise one or more of human milk cream or sub-fractions thereof, human skim milk, human milk permeate, or human milk retentate, wherein the human milk permeate and the human milk retentate are obtained from filtration of human skim milk. In some embodiments, the topical formulation comprises human milk cream. In certain embodiments, the topical formulation comprises a sub-fraction obtained or derived from human milk cream. In some embodiments, the sub-fraction is butteroil. In certain embodiments, the sub-fraction is a milk fat globule membrane solids sub-fraction. In some embodiments, the topical formulation comprises human milk fatty acids.

In some embodiments, the topical formulation comprises one or more of oleic acid, palmitic acid, linoleic acid, omega 3 fatty acid, omega 6 fatty acid, omega 9 fatty acid, stearic acid, myristic acid, lauric acid, palmitoleic acid, vaccenic acid, myristolic acid, heptadecenoic acid, gamma linolenic acid, docosapentaenoic acid, eicosapentaenoic acid, arachidonic acid, and docosahexaenoic acid. In some embodiments, wherein the topical formulation comprises oleic acid, palmitic acid, linoleic acid, omega 3 fatty acid, omega 6 fatty acid, omega 9 fatty acid, stearic acid, myristic acid, lauric acid, palmitoleic acid, vaccenic acid, myristolic acid, heptadecenoic acid, gamma linolenic acid, docosapentaenoic acid, eicosapentaenoic acid, arachidonic acid, and docosahexaenoic acid.

In certain embodiments, the topical formulation comprises human milk permeate, wherein the human milk permeate is obtained from filtration of human skim milk. In some embodiments, the filtration is or comprises ultra-filtration. In some embodiments, the topical formulation comprises human milk fatty acids and human milk permeate. In particular embodiments, the topical formulation comprises a mixture of human milk oligosaccharides, wherein the mixture of human milk oligosaccharides comprises two or more different HMOs.

In certain embodiments, the topical formulation comprises from 1% to 30% or 1% to 40% human milk fat, fatty acids, or lipids as a percent of fat, fatty acid, or lipid weight over total weight (w/w). In particular embodiments, the topical formulation comprises from 5% to 25% human milk fat, fatty acids, or lipids (w/w). In some embodiments, the topical formulation comprises less than or equal to 30%, 25%, or 10% human milk fat, fatty acids, or lipids (w/w). In some embodiments, the topical formulation comprises between 1% and 10% human milk fat, fatty acids, or lipids (w/w). In certain embodiments, the topical formulation comprises at least or about 10% human milk fat, fatty acids, or lipids (w/w). In particular embodiments, the topical formulation comprises at least or about 20% human milk fat, fatty acids, or lipids (w/w). In certain embodiments, the topical formulation comprises at least or about 30% human milk fat, fatty acids, or lipids (w/w). In some embodiments, the topical formulation comprises one or more human milk oligosaccharides.

Also provided herein is a topical formulation comprising human milk cream and human milk permeate, wherein the topical formulation comprises between 1% and 10% or from 1% to 40% or 10% to 40% human milk fat, lipids, or fatty acids (w/w), wherein the human milk permeate is obtained from the ultra-filtration of human skim milk. In some embodiments, the topical formulation comprises at least 0.3% human milk oligosaccharides (w/w). In some embodiments, the topical formulation comprises at least 0.3% human milk oligosaccharides (w/w). In some embodiments, the topical formulation comprises between 0.1% and 0.5% human milk oligosaccharides (w/w).

In some embodiments, the topical formulation comprises a mixture of at least two human milk oligosaccharides. In certain embodiments, the topical formulation comprises a mixture of at least 25, 50, 75, or 100 human milk oligosaccharides. In some embodiments, the topical formulation comprises one or more of 2′-fucosyl-lactose, 3-fucosyl-lactose, 3′-sialyl-lactose, 6′-sialyl-lactose, lacto-N-tetraose, lacto-N-difucohexaose I, lactodifucotetraose, lacto-N-fucopentaose I, sialylacto-N-tetraose c, sialylacto-N-tetraose b, and disialyllacto-N-tetraose. In some embodiments, the topical formulation comprises 2′-fucosyl-lactose, 3-fucosyl-lactose, 3′-sialyl-lactose, 6′-sialyl-lactose, lacto-N-tetraose, lacto-N-difucohexaose I, lactodifucotetraose, lacto-N-fucopentaose I, sialylacto-N-tetraose c, sialylacto-N-tetraose b, and disialyllacto-N-tetraose.

In some embodiments, the topical formulation comprises human milk retentate, wherein the human milk retentate is obtained from filtration of human skim milk. In some embodiments, the filtration is or comprises ultra-filtration. In some embodiments, the topical formulation comprises one or more human milk proteins. In particular embodiments, the topical formulation comprises one or more of casein, α-lactalbumin, lactoferrin, secretory immunoglobulin IgA, lysozyme, serum albumin, lactadherin, dermcidin, immunoglobulins, cytokines, mucins, or growth factors.

In some embodiments, the topical formulation comprises between 0.1% and 0.5% human milk oligosaccharides (w/w) and between 1% and 30% human milk fat, lipids, or fatty acids (w/w). In certain embodiments, the topical formulation comprises about 0.3% % human milk oligosaccharides (w/w) and about 20% human milk fat, fatty acids, or lipids (w/w). In some embodiments, the topical formulation comprises about 0.3% % human milk oligosaccharides (w/w) and about 10% human milk fat, lipids, or fatty acids (w/w). In some embodiments, the one or more excipients comprise one or more of a hydrocolloid, a thickening agent, an antioxidant, a preservative, or a gelling agent.

In some embodiments, the topical formulation is or comprises an oil-in water emulsion, a water-in-oil emulsion, a nanoemulsion, an emulsified gel, an oleaginous ointment, a hydrocolloid system, or a cream preparation. In some embodiments, the topical formulation is or comprises an oil-in-water emulsion.

Provided herein is a method of treating a disorder or condition, comprising administering any of the topical formulations described herein to a subject in need thereof. In some embodiments, the subject displays or is at risk of displaying one or more symptoms associated with a skin condition or disorder. In some embodiments, the application comprises applying the topical formulation locally to a region of skin displaying or at risk of displaying the one or more symptoms.

In some embodiments, the skin condition or disorder comprises one or more of dermatitis, atopic dermatitis, contact dermatitis, hand dermatitis, allergic dermatitis, gravitational dermatitis, asteatotic dermatitis, nummular dermatitis, seborrhoeic dermatitis, infective dermatitis, acne, acne vulgaris, rosacea, epidermolysis bullosa simplex, chronic superficial scaly acrodermatitis, chondrodermatitis, perioral dermatitis, dermatomyositis, neutrophilic dermatosis, transient acantholytic dermatosis, eczema, ichthyosis, xerosis, asteatosis, psoriasis, lupus erythematosus, exfoliative keratolysis, impetigo, allergic inflammation of the skin, urticaria, laceration, burn, or a wound resulting from a surgical incision or a biopsy. In some embodiments, the skin condition or disorder comprises psoriasis. In some embodiments, the skin condition or disorder comprises atopic dermatitis.

In particular embodiments, the one or more symptoms associated with the skin condition or disorder comprise one or more of dryness, lesions, cracks, fissures, scaling, discoloration, thickening, redness, bleeding, swelling, flaking, ulcers, sores, wounds, blistering, rashes, inflammation, infection, or loss of flesh. In some embodiments, administering the topical formulation comprises locally or directly applying the topical formulation to a region of skin having or at risk of having the one or more symptoms.

Provided herein is a method for reducing or preventing inflammation in a subject in need thereof, comprising applying a topical formulation described herein locally or directly to a region on the subject's skin or in the subject's mouth that is or is at risk of being inflamed. In some embodiments, the one or more symptoms associated with the skin condition or disorder comprise inflammation.

In some embodiments, provided herein is a method of treating a wound comprising applying the topical formulation of any of claims 1-29 to a subject in need thereof locally at a wound, optionally to promote healing or reduce probability or degree of scarring. In some embodiments, the subject is or is at risk of experiencing pain associated with breastfeeding. In some embodiments, the administration comprises applying the topical formulation to the subject's breast or nipple.

Provided herein is a method of reducing transepidermal water loss in a subject in need thereof, comprising applying a topical formulation described herein to a region on the subject's skin. In some embodiments, the subject displays or is at risk of displaying one or more symptoms associated with mucositis. In certain embodiments, the one or more symptoms associated with mucositis comprises one or more of pain, inflammation, or ulceration. In some embodiments, the administration comprises oral administration of the topical formulation.

In some embodiments, provided herein is a use of the topical formulation in the treatment or prevention of one or more symptoms associated with a skin condition or disorder. In some embodiments, provided herein is a use of a topical formulation described herein in the manufacture of a medicament for the treatment or prevention of one or more symptoms associated with a skin condition or disorder. In some embodiments, provided herein is a method for improving the appearance of damaged or aged skin, comprising administering a topical formulation described herein to a subject in need thereof.

In some embodiments, administering the topical formulation comprises locally or directly applying the topical formulation to the aged or damaged skin. In some embodiments, the aged or damaged skin comprises one or more of inflammation, irritation, and erythema of the skin, wrinkles, skin blemishes, “marionette” lines, smile lines, deep nasolabial fold lines, crow's feet, fine lines, vertical lines between the eyebrows, horizontal forehead lines, sagging thin/frail skin, dryness, dullness, loss of elasticity, lack of radiance, appearance of spider vessels or red blotchiness, exaggerated lines, and wrinkles.

Provided herein is a method of making a topical formulation comprising formulating one or more human milk fractions with one or more excipients, e.g., pharmaceutically acceptable excipients, wherein the one or more human milk fractions comprise one or more of human milk cream, human milk butteroil, human milk fat globule membrane solids, human skim milk, human milk permeate, and human milk retentate, wherein the human milk permeate and the human milk retentate are obtained from filtration of human skim milk; thereby making a topical formulation.

In some embodiments, the human milk fractions are obtained from a pool of human milk collected from multiple human milk donors. In some embodiments, the pool of human milk comprises human milk collected from at least 5, 10, 25, 50, or 100 human milk donors. In some embodiments, the one or more human milk fractions comprises human milk cream or sub-fractions obtained from or derived from human milk cream, e.g., human milk butteroil or milk fat globule membrane solids. In some embodiments, the topical formulation is formulated with about or at least 1%, 3%, 5%, 10%, or 20% human milk cream or human milk butteroil. In some embodiments, the topical formulation is formulated with less than 75%, 60%, or 50% human milk cream or human milk butteroil. In certain embodiments, the topical formulation is formulated with from 20% to 70% human milk cream or human milk butteroil. In particular embodiments, the human milk cream or butteroil is mixed in an amount sufficient to provide a final concentration of from 1% to 30% human milk fat, fatty acids, or lipids (w/w). In some embodiments, the amount of human milk cream or butteroil is sufficient to provide a final concentration of from 5% to 25% human milk fat, fatty acids, or lipids (w/w). In certain embodiments, the amount of human milk cream or butteroil is sufficient to provide a final concentration of about 20% human milk fat, fatty acids, or lipids (w/w). In particular embodiments, the amount of human milk cream or butteroil is sufficient to provide a final concentration of about 10% human milk fat, fatty acids, or lipids (w/w).

In some embodiments, the one or more human milk fractions comprises human milk permeate obtained from filtration of human skim milk. In some embodiments, the filtration is or comprises ultra-filtration. In some embodiments, the permeate comprises at least 0.1%, 03%, 0.5% or 1.0% human milk oligosaccharides (w/w). In some embodiments, prior to mixing the permeate with the one or more excipients, the permeate undergoes one or more steps for concentrating human milk oligosaccharide content to at least 0.5%, 1%, 3%, 5%, or 10% human oligosaccharides (w/w). In some embodiments, the one or more steps for concentrating human milk oligosaccharide content comprises one or both of nanofiltration or reverse osmosis.

In some embodiments, prior to mixing the permeate with the one or more excipients, the permeate undergoes one or more steps to reduce the lactose content. In some embodiments, the one or more steps to reduce lactose content comprises (i) contacting the permeate with a lactase enzyme to produce a permeate/lactase mixture, (ii) incubating the permeate/lactase mixture at a temperate of between 45° C. and 55° C., and (iii) filtering the permeate/lactase mixture to remove at least 90% of the lactase from the permeate, thereby reducing the lactose content of the permeate.

In some embodiments, the topical formulation is formulated with at least 5%, 10%, or 25% permeate obtained from the ultra-filtration of human skim milk. In some embodiments, the topical formulation is formulated with less than 90%, 80%, or 75% permeate obtained from the ultra-filtration of human skim milk. In some embodiments, the human milk permeate is mixed in an amount sufficient to provide a final concentration of at least 0.1% human milk oligosaccharides (w/w). In certain embodiments, the amount of the human milk permeate sufficient to provide a final concentration of at least 0.3% human milk oligosaccharides (w/w). In particular embodiments, the amount of the human milk permeate sufficient to provide a final concentration of from 0.1% and 0.5% human milk oligosaccharides (w/w). In some embodiments, the amount of the human milk permeate sufficient to provide a final concentration of from 0.3% human milk oligosaccharides (w/w). In certain embodiments, the topical formulation is formulated with between 1% and 60% human milk cream or human milk butteroil and between 25% and 80% human milk permeate obtained from the ultra-filtration of human skim milk. In particular embodiments, the topical formulation is formulated with between 20% and 60% human milk cream or butteroil and between 25% and 80% human milk permeate obtained from the ultra-filtration of human skim milk.

In some embodiments, the one or more excipients comprise one or more of a hydrocolloid, an emulsifier, a structuring agent, an antioxidant, a silicone containing compound, a thickening agent, a preservative, an additional skin conditioning agent, an emollient, a humectant, or an essential oil. In some embodiments, the topical formulation is or comprises an oil-in water emulsion, a water-in-oil emulsion, a nanoemulsion, an emulsified gel, an oleaginous ointment, a hydrocolloid system, or a cream preparation.

Provided herein is a method of making an oil-in-water emulsion comprising one or more human milk fractions, the method comprising: combining an aqueous phase and an oil phase to produce an oil-in-water emulsion, wherein (i) the aqueous phase comprises water, human milk cream or a sub-fraction thereof, e.g., a human milk butteroil or a human milk fat globule membrane solid sub-fraction, and one or more excipients that are hydrophilic or soluble in water and wherein (ii) the oil phase comprises one or more excipients that are hydrophobic or soluble in oil; thereby making an oil-in-water emulsion comprising one or more human milk fractions.

Provided herein is a method of making an oil-in-water emulsion comprising one or more human milk fractions, the method comprising: combining an aqueous phase and an oil phase to produce an oil-in-water emulsion, wherein (i) the aqueous phase comprises human milk permeate and one or more excipients that are hydrophilic or soluble in water, wherein the human milk permeate is obtained from the ultra-filtration of human skim milk; and wherein (ii) the oil phase comprises one or more excipients that are hydrophobic or soluble in oil; thereby making an oil-in-water emulsion comprising one or more human milk fractions.

In some embodiments, the aqueous phase comprises human milk cream or a sub-fraction thereof, e.g., human milk butteroil. In some embodiments, one or more thickening agents are added to the oil-in-water emulsion. In some embodiments, human milk cream or sub-fraction thereof is added to the oil-in-water emulsion after combining the aqueous phase and the oil phase. In some embodiments, the aqueous phase comprises one or more of an emulsifying agent, an antimicrobial agent, and a humectant. In some embodiments, the oil phase comprises one or more of an emollient, an emulsifying agent, and an antioxidant.

Provided herein is a method of making an oil-in-water emulsion comprising one or more human milk fractions comprising; combining an aqueous phase and an oil phase to produce an oil-in-water emulsion, wherein (i) the aqueous phase comprises an emulsifying agent, an antimicrobial agent, and human milk permeate, wherein the human milk permeate is obtained from an ultra-filtration of human skim milk; (ii) the oil phase comprises one or more excipients, e.g., pharmaceutically acceptable excipients, that are hydrophobic or soluble in oil, thereby making an oil-in-water emulsion; adding human milk cream or a sub-fraction thereof to the oil-in-water emulsion; and adding a thickening agent to the oil-in-water emulsion; thereby making an oil-in-water emulsion comprising one or more milk fractions. In some embodiments, the oil-in-water emulsion comprising one or more milk fractions comprises: between 3% and 30% human milk fat, fatty acids, or lipids (w/v). In certain embodiments, the oil-in-water emulsion comprises about 20% human milk fat, fatty acids, or lipids (w/v). In some embodiments, the oil-in-water emulsion comprises about 10% human milk fat, fatty acids, or lipids (w/v).

### BRIEF DESCRIPTION OF THE DRAWINGS

FIGS. 1A and 1B depict a three-dimensional in vitro human skin model. A schematic of the cell culture process is shown in FIG. 1A. A stained section of three-dimensional in vitro skin model with labels indicating cell layers corresponding to human skin is shown in FIG. 1B.

FIG. 2 shows skin barrier function as a percentage of the transepithelial/transendothelial electrical resistance (TEER) as measured in Ohm*cm² at 1,000 Hz following in a three-dimensional in vitro human skin model incubated with vehicle containing PBS with 1% DMSO (D); human milk cream and permeate (PC); 30% cream (CI 30%); 1% human milk permeate (P 1%), or 30% human milk retentate (UF 30%).

FIG. 3 shows levels of IL-8 measured in media of three-dimensional human skin models following incubation in an untreated control group (NC) or following treatment with TNF-alpha and incubation with a vehicle containing PBS with 1% DMSO (D), 1% human milk cream (CI 1%), 5% permeate (P 5%), or 1% human milk retentate (UF).

FIG. 4 shows barrier function three-dimensional human skim models as a percentage of TEER as compared to measurements collected from vehicle only controls prior to prior to mechanical disruption. Barrier function is shown before and immediately after or 3, 5, or 8 days following a mechanical disruption in cell culture models incubated in PBS containing 1% DMSO (D), a combination of human milk cream and permeate (PC), or 30% skim milk (SM30%).

FIG. 5 shows TEER measurements collected from three-dimensional skin models incubated with in a vehicle containing PBS with 1% DMSO (D) or 10 human milk retentate (10% UF) before, immediately after (After), and at various time points after a dermal punch removal.

FIGS. 6A-C show levels of IL-8 measured in media of three-dimensional human skin models in an untreated control (untreated) or following treatment with TNF-alpha and a 24-hour (FIG. 6A) and 72 hour (FIG. 6B) incubation with a vehicle oil-in-water emulsion or oil-in-water emulsions containing a positive control, human milk cream, human milk permeate, or both human milk cream and permeate. IL-8 levels measured 48 hours after removal of the emulsions are shown in FIG. 6C.

### DETAILED DESCRIPTION

Provided herein are compositions, e.g., topical formulations such as ointments, skin creams, lotions, or emulsions, that contain or include milk, e.g., human milk, or a fraction, portion, or component thereof. In some embodiments, the milk is human milk. In certain embodiments, the composition is or includes a topical formulation containing a fraction of human milk. In particular embodiments, the composition is a topical formulation that is or includes one or more of human milk cream, human skim milk, or permeate or retentate resulting from the filtration of human skim milk (also referred to herein as human milk permeate or human milk retentate, respectively). Also provided are methods for manufacturing compositions, e.g., topical formulations, containing one or more human milk fractions, portions, or components, as well as methods of use for the provided compositions to treat or prevent a skin condition or disorder.

Currently there are many treatments for skin conditions and disorders ranging from special diets to emollients and immunosuppressive ointments e.g., corticosteroid ointments. While corticosteroids, such as hydrocortisone or clobetasol propionate (topical, oral, or intradermal administration) may bring about improvements, they also often have side effects. Prolonged use is thought to increase the risk of these side effects, the most common of which is the skin becoming thin and fragile (atrophy). Because of this, if used on delicate skin, a low-strength steroid should be used or applied less frequently. Additionally, high-strength steroids used over large areas, or under occlusion, may be absorbed into the body, causing hypothalamic-pituitary-adrenal axis suppression (HPA axis suppression) and all of the side effects normally associated with systemic steroid use.

An increased risk of skin infections with bacteria such as Staphylococcus aureus or fungi may result from skin conditions or disorders such as atopic dermatitis or eczema. Therefore, in more severe cases, dermatologists may also prescribe either topical or oral conventional antibiotics such as penicillin, streptomycin, and chloramphenicol. While the effectiveness of such treatment may vary from person to person, there are well-known disadvantages of conventional antibiotics, such as a-specificity, e.g., toxicity to nonpathogenic or beneficial bacteria, and the risk of resistance, not only by the target bacteria but also by other pathogenic bacteria. Furthermore, conventional, systemic antibiotic treatment can interact with other drugs, and certain antibiotics cannot be combined with the use of alcohol. Accordingly, there is a need for skin treatments that avoid such drawbacks, while promoting effective wound healing and/or reducing inflammation.

The provided compositions address these needs. In some aspects, the provided compositions improve barrier function, promote healing and regeneration, and/or reduce inflammation in human skin with a high degree of effectiveness. In certain aspects, the provided compositions contain natural human lipids, oligosaccharides, or proteins, and therefore reduce likelihood of sensitization, irritation, or allergy, for example as compared to skin treatments containing active ingredients that are non-human in origin. In certain aspects, the provided compositions reduce symptoms associated with inflammation while also strengthening the skin, such as by improving the barrier function.

Human milk contains a complex mixture of bioactive components including, lipids, vitamins, amino acids, oligosaccharides, sugars, cofactors, nucleotides, amino acids, and energy intermediates, each of which may benefit aspects of skin health. The provided compositions include formulations that incorporate some or all of these components into formulations, e.g., topical formulations that allow for localized and/or direct application to human skin. Unlike currently available skin treatments, the provided compositions may contain natural human lipids, proteins, and oligosaccharides that support skin health while minimizing adverse effects. For example, in some aspects, the human origin of the active ingredients reduces the probability or likelihood of adverse effects arising from sensitization or allergic reactions.

The provided compositions are or include topical formulations that may be applied to damaged skin, for example to a region exhibiting one or more symptoms associated with a skin disease or disorder. In some aspects, the provided compositions are especially useful for reducing skin inflammation while also strengthening the barrier function of the skin. Such effects are, in some aspects, beneficial over alternative skin treatments such as corticosteroids that may reduce inflammation but weaken barrier function.

In addition to boosting the immune functions of infants, human milk contains antimicrobial and antibacterial properties as well. For example, in certain aspects, human milk may contain antibodies and oligosaccharides that may kill or prevent the growth of pathogenic bacteria or otherwise promote a healthy skin microbiome. In some aspects, antimicrobial human milk components are formulated into a stable composition that may be administered or applied to a subject, e.g., topically, to treat or prevent infection without any unwanted side effects that are associated with acute or prolonged treatment with antibiotics.

In certain aspects, sensitization may be a concern with some existing skin treatments. In some aspects, skin sensitization may manifest as allergic response to a substance after skin contact, such as in the form of allergic contact dermatitis (ACD). In some estimates, up to about 20% of the general population is sensitive to at least one allergen (Thyssen et al., Contact Dermatitis 2007; 57: 287-299.). ACD is the clinical condition caused by an allergic immune response following skin exposure to a large subset of small reactive chemicals (haptens) that can be found in the environment and in many household products. In certain embodiments, the provided compositions and formulations are formulated or composed with proteins, lipids, oligosaccharides, or other macromolecules that are natural and of human origin. In particular aspects, such formulations reduce the possibility of sensitization and irritation to the skin as compared to alternative skin treatments, particularly those containing natural plant extracts and/or non-human, animal based constituents.

In particular aspects, the provided topical formulations include one or more ingredients, components, portions, or fractions from human milk. In some aspects, the topical formulations have advantages over alternative topical formulations that may contain certain compounds such as lipids, fats, fatty acids, proteins, or oligosaccharides that that may be present in human milk but are synthesized or derived from non-human milk sources. Such advantages may include anti-inflammatory activity or promotion skin of skin repair to a greater degree than what might be observed from these alternative topical formulations. In some such embodiments, the combinations of compounds such as (but not limited to) lipids, fats, fatty acids, proteins, and/or oligosaccharides found in topical formulations derived from or formulated with human milk fractions or components contain a variety of human milk compounds, e.g., that may act synergistically, to provide such advantages, e.g., anti-inflammation or skin repair, over the alternative topical formulations.

In some aspects, the provided compositions, e.g., topical formulations, overcome difficulties that may be associated with formulating compositions containing cream from human milk. For example, in some aspects, compositions containing human milk cream may be prone to spoilage, phase separation, denaturization, or discoloration (such as discoloration that indicates breakdown of macromolecules, proteins, oligosaccharides, or lipids) when it is prepared in a typical emulsion, such as those containing excipients that may be suitable for formulating creams or lipids from other sources, e.g., bovine or plant sources. In particular embodiments, the provided compositions and methods overcome such problems with specific formulations tailored to stabilize compositions containing human milk cream, such as oil-in-water emulsions, lotions, creams, or ointments. Such formulations result in stable formulations that do not spoil, discolor, or undergo phase separation for weeks, months, or even years.

All publications, including patent documents, scientific articles, and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications, and other publications that are herein incorporated by reference, the definition set forth herein prevails over the definition that is incorporated herein by reference.

The section headings used herein are for organizational purposes only and are not to be construed as limiting the subject matter described.

### I. Topical Formulations Containing Human Milk Fractions, Portions, or Components

Provided herein are compositions that are or include topical formulations that may be locally and/or directly applied to the skin or mucous membranes. In some embodiments, the topical formulations may be applied to the skin for cosmetic purposes. In certain embodiments, the topical formulations may be applied locally and/or directly to the skin or mucous membranes to treat or prevent a disease or disorder. In certain embodiments, the topical formulations include at least one ingredient that is derived or originates from milk. In some embodiments, the topical formulation includes one or more of a portion, fraction, or component that is derived or originates from human milk. In particular embodiments, milk or portions, fractions, or components thereof are formulated into topical formulations, such as by mixing, combining, or formulating with one or more pharmaceutically acceptable excipients.

In some embodiments, the milk is a mammalian milk. In particular embodiments, the milk is or includes cow milk, donkey milk, goat milk, sheep milk, camel milk, mare's milk, or buffalo milk. In some embodiments, the milk is or includes cow milk. In particular embodiments, the milk is or includes camel milk. In various embodiments, the topical formulations include one or more ingredients that originate or are derived from human milk. In particular embodiments, the topical formulations include human milk or portions, fractions, or components thereof.

In certain embodiments, the topical formulation is or includes whole human milk. In some embodiments, the topical formulation is or includes at least 1%, 5%, 10%, 20%, 25%, 30%, 40%, 50%, 60%, 70%, 75%, or 80% or more whole human milk. In certain embodiments, topical formulation contains no more than 75%, 70%, 60%, 50%, 40%, 30%, 25%, 20%, or 10% whole human milk. In some embodiments, the topical formulation contains between or between about 1% and 75%, 5% and 50%, 10% and 50%, 10% and 30%, 10% and 20%, 30% and 50%, 40% and 60%, or 50% and 75% whole human milk, e.g., by weight/weight (w/w) or weight/volume (w/v).

Human Milk Cream

In some embodiments, the topical formulation is formulated with cream, e.g., human milk cream. In some embodiments, the topical formulation is or includes cream, e.g., cream obtained from or separated from whole milk. In particular embodiments, the topical formulation is or includes human milk cream (also referred to herein as human cream or cream from human milk). In some embodiments, the composition contains a portion or fraction of human milk cream (also referred to herein as a sub-fraction of human milk cream), e.g., a portion or component of the human milk cream that has been isolated or concentrated from human milk cream.

In some embodiments, the cream, e.g., human milk cream, is obtained from a method or technique that separates human milk cream from human skim milk. Particular embodiments contemplate that methods for separating cream from skim may be performed as a matter of routine by any known method in the art. In some embodiments, the cream is separated from skim by a method or technique that is or includes creaming separation or centrifugation, such as any of those described herein e.g., in Section III. In some embodiments, the human milk cream is a or includes a human milk fraction that contains a high concentration of fat, e.g., from 20% to 60% fat w/v or w/w that is separated from a human skim milk fraction of whole human milk.

In particular embodiments, the human milk cream contains only human milk components, e.g., lipids, fatty acids, fats, carbohydrates, sugars, oligosaccharides, or proteins that are concentrated, isolated, or derived from, native to, or found in human milk. In particular embodiments, the human milk cream is free or essentially free of non-human lipids, fatty acids, fats, carbohydrates, sugars, oligosaccharides, or proteins, e.g., lipids, fatty acids, fats, carbohydrates, sugars, oligosaccharides, or proteins that are not concentrated, isolated, or derived from, native to, or found in human milk. In certain embodiments, the human milk cream is free or essentially free of xenogeneic components or compounds, such as including but not limited to lipids, fatty acids, fats, carbohydrates, sugars, oligosaccharides, or proteins.

In some embodiments, the human milk cream is or includes from about 20% to 60% human milk lipids, fat, or fatty acids, e.g., as a percent by weight/weight (w/w). In various embodiments, the human milk cream is a milk fraction that is separated from whole milk by or during the process for generating human skim milk from human whole milk, e.g., by centrifugation, and, in certain embodiments, contains between or between about 20% and 60% human milk lipids, fat, or fatty acids, e.g., w/w. In some embodiments, the human milk cream is or includes between 30% and 70%, 40% and 65%, 45% and 65%, 48% and 62%, or 50% and 60% lipids, fat or fatty acids, e.g., w/w.

In certain embodiments, the topical formulation is, includes, or is formulated with at least 1%, 5%, 10%, 20%, 25%, 30%, 40%, 50%, 60%, 70%, 75%, or 80% or more human milk cream, e.g., w/w or v/w. In certain embodiments, the topical formulation is or includes no more than 75%, 70%, 60%, 50%, 40%, 30%, 25%, 20%, or 10% human milk cream. In some embodiments, the topical formulation contains between or between about 1% and 75%, 5% and 50%, 10% and 50%, 10% and 30%, 10% and 20%, 30% and 50%, 40% and 60%, or 50% and 75%, human milk cream, each inclusive.

In certain embodiments, the topical formulation contains between or between about 10% and 30% human milk cream, e.g., w/v or v/v. In certain embodiments, the topical formulation contains between or between about 50% and 75% human milk cream. In certain embodiments, topical formulation contains or contains about 10% human milk cream. In some embodiments, the topical formulation contains or contains about 30% human milk cream. In particular embodiments, the topical formulation contains or contains about 50% human milk cream.

In particular embodiments, an amount of human milk cream is mixed, added, or incorporated into a topical formulation. In certain embodiments, the amount is sufficient to achieve a target amount or content of human milk lipids, fat, or fatty acids. In certain embodiments, the human milk cream is mixed, added, or incorporated into a topical formulation in an amount sufficient to achieve a topical formulation containing a human milk lipid, fat, or fatty acid content of at least 1%, 2%, 3%, 4%, 5%, 10%, 15%, 20%, 25%, or 30%, or between 1% and 40%, 5% and 30%, 10% and 30%, 5% and 15%, or 7.5% and 12.5%, each inclusive, e.g., w/w or w/v. In some embodiments, an amount of human milk cream is mixed, added, or incorporated to achieve a topical formulation containing about 10% human milk fat, e.g., w/w or w/v. In certain embodiments, an amount of human milk cream is mixed, added, or incorporated to achieve a topical formulation containing about 20% human milk fat, e.g., w/w or w/v. In particular embodiments, an amount of human milk cream is mixed, added, or incorporated to achieve a topical formulation containing about 30% human milk fat, e.g., w/w or w/v.

In some embodiments, the topical formulation is free or essentially free of non-human lipids. In certain embodiments, the topical formulation is free or essentially free of mammalian milk lipids that are not human milk lipids.

In particular embodiments, the topical formulation contains or includes one or more milk lipids, fat, or fatty acids. In certain embodiments, the topical formulation contains or includes at least 1%, 2%, 3%, 4%, 5%, 10%, 15%, 20%, 25%, 30%, 35%, or 40% human milk lipids, e.g. w/w or w/v. In certain embodiments, the topical formulation contains or includes at least 1%, 2%, 3%, 4%, 5%, 10%, 15%, 20%, 25%, 30%, 35%, or 40% human milk fat, e.g. w/w or w/v. In certain embodiments, the topical formulation contains or includes at least 1%, 2%, 3%, 4%, 5%, 10%, 15%, 20%, 25%, 30%, 35%, or 40% human milk fatty acids, e.g. w/w or w/v. In particular embodiments, the topical formulation contains between 1% and 60%, 1% and 40%, 25% and 50%, 5% and 30%, 10% and 30%, 5% and 15%, or 7.5% and 12.5% human milk lipids, fat, or fatty acids, each inclusive, e.g., w/w or w/v. In some embodiments, the topical formulation contains between 1% and 60%, 25% and 50%, 5% and 30%, 5% and 15%, or 7.5% and 12.5% human milk fat, lipids, or fatty acids, each inclusive, e.g., w/w or w/v.

In particular embodiments, the topical formulation contains one or more human milk lipids, e.g., lipids that are found in or native to human milk. In some embodiments, the topical formulation contains one or more neutral complex lipids (e.g., free fatty acids, cholesteryl esters, diacylglycerols, triacylglycerols, monoacylglycersols), sphingolipids (e.g., ceramides, dihyroceramindes, hexosylceramides, lactosylceramides, sphingomyelins), phospholipids (e.g., phosphatidylcholines, lysophosphatidylcholines, phosphatidylethanolamines, lysophophatidylethanolamines, phosphatidylinositols), or a combination any or all of the foregoing, that are found in or native to human milk.

In certain embodiments, the topical formulation contains one or more human lipids that are found in or native to human stratum corneum. In some embodiments, the composition, e.g., topical composition, contains one or more neutral complex lipids (e.g., free fatty acids, cholesteryl esters, diacylglycerols, triacylglycerols, monoacylglycersols), sphingolipids (e.g., ceramides, dihyroceramindes, hexosylceramides, lactosylceramides, sphingomyelins), phospholipids (e.g., phosphatidylcholines, lysophosphatidylcholines, phosphatidylethanolamines, lysophophatidylethanolamines, phosphatidylinositols), or a combination any or all of the foregoing, that are found in or native to human stratum corneum.

In certain embodiments, the topical formulation comprises one or more fatty acids that are concentrated, isolated, or derived from human milk or are native to or found in human milk or human milk cream. In some embodiments, the human milk oligosaccharides include, but are not limited to oleic acid, palmitic acid, linoleic acid, omega 3 fatty acid, omega 6 fatty acid, omega 9 fatty acid, stearic acid, myristic acid, lauric acid, palmitoleic, vaccenic acid, myristolic acid, heptadecenoic acid, gamma linolenic acid, docosapentaenoic acid, eicosapentaenoic acid (EPA), arachidonic acid (AA) and docosahexaenoic acid (DHA).

In certain embodiments, the topical formulation is or includes at least 1%, 2%, 3%, 4%, 5%, 10%, 15%, 20%, 25%, or 30% human milk lipids, fat, or fatty acids. In some embodiments, the topical formulation is or includes at least 1%, 2%, 3%, 4%, 5%, 10%, 15%, 20%, 25%, or 30% human milk fat. In certain embodiments, the topical formulation is or includes between 20% and 50%, 1% and 40%, 5% and 30%, 10% and 30%, 5% and 15%, or 7.5% and 12.5% human milk lipids, fat, or fatty acids, each inclusive, e.g., w/w or w/v. In some embodiments, the topical formulation is or includes between 5% and 30%, 5% and 15%, or 7.5% and 12.5% human milk fat, e.g., w/w. In certain embodiments, the topical formulation is or includes about 10% human milk fat, e.g., w/w. In particular embodiments, the topical formulation is or includes about 20% human milk fat, e.g., w/w. In certain embodiments, the topical formulation is or includes about 30% human milk fat, e.g., w/w.

In some embodiments, the topical formulation is formulated with a sub-fraction obtained or derived from human milk cream. In certain embodiments, the topical formulation is formulated with human milk butteroil. In certain embodiments, the human milk butteroil results from removal of some or all of the milk solids and water from the human milk cream. In particular embodiments, the butteroil is anhydrous milk fat. In some embodiments, the human milk butteroil is a fraction of human milk that contains at least 80%, 85%, 90%, 95%, 98%, 99%, or 99.8% human milk fat, fatty acids, or lipids. Particular embodiments contemplate that a butteroil sub-fraction of human milk cream may be obtained or derived from human milk cream by any known suitable method as a matter of routine.

In particular embodiments, the topical formulation is, includes, or is formulated with at least 1%, 5%, 10%, 20%, 25%, 30%, 40%, 50%, 60%, 70%, 75%, or 80% or more human milk butteroil, e.g., w/w or w/v. In certain embodiments, the topical formulation is or includes no more than 75%, 70%, 60%, 50%, 40%, 30%, 25%, 20%, or 10% human milk butteroil. In some embodiments, the topical formulation contains from 1% to 60%, 10% and 30%, 30% and 60%, or 20% and 40% human milk butteroil w/w or w/v.

In some embodiments, an amount of human milk butteroil is mixed, added, or incorporated into a topical formulation. In certain embodiments, the amount is sufficient to achieve a target amount or content of human milk lipids, fat, or fatty acids. In certain embodiments, the butteroil is mixed, added, or incorporated into a topical formulation in an amount sufficient to achieve a final concentration of human milk lipid, fat, or fatty acid of about or at least 1%, 5%, 10%, 20%, 30%, or 40%, or from 5% to 50% or 10% to 30%, e.g., w/w or w/v. In particular embodiments, the human milk butteroil is added to achieve a topical formulation containing about 10% human milk fat. In certain embodiments, the human milk butteroil is added to achieve a topical formulation containing about 20% human milk fat. In some embodiments, the human milk butteroil is added to achieve a topical formulation containing about 30% human milk fat.

In some embodiments, topical formulation is formulated with a sub-fraction of human milk cream that is or includes human milk fat globule membrane solids. In some embodiments, the human milk fat globule membrane solids sub-fraction is obtained by any suitable known means, including but not limited to methods described herein, e.g., in Section III and/or in Example 17.

In particular embodiments, the topical formulation is, includes, or is formulated with at least 1%, 5%, 10%, 20%, 25%, 30%, 40%, 50%, 60%, 70%, 75%, or 80% or more w/w or w/v human milk fat globule membrane solids obtained or derived from human milk cream. In certain embodiments, the topical formulation is or includes no more than 75%, 70%, 60%, 50%, 40%, 30%, 25%, 20%, or 10% human milk fat globule membrane solids obtained or derived from human milk cream. In some embodiments, the topical formulation contains from 1% to 60%, 1 to 40%, 10% to 30%, 30% to 60%, or 20% to 40%, w/w or w/v.

In some embodiments, an amount of human milk fat globule membrane solids derived or obtained from human milk cream, e.g., by centrifugation, is mixed, added, or incorporated into a topical formulation. In certain embodiments, the amount is sufficient to achieve a target amount or content of human milk lipids, fat, or fatty acids. In certain embodiments, the human milk fat globule membrane solids is mixed, added, or incorporated into a topical formulation in an amount sufficient to achieve a final concentration of human milk lipid, fat, or fatty acid of about or at least 1%, 5%, 10%, 20%, 30%, or 40%, or from 5% and 50% or 10% to 30%, e.g., w/w or w/v.

Human Skim Milk

In some embodiments, the topical formulation is formulated with skim milk. In particular embodiments, the topical formulation is formulated with human skim milk. In certain embodiments, the skim milk is obtained from a method or technique that is or includes centrifugation or creaming separation. In some embodiments, the skim may be further processed, e.g., with additional centrifugations, to further remove fat globules, lipids, and/or fatty acids from the skim.

In some embodiments, the topical formulation contains human skim milk. In certain embodiments, the topical formulation contains, contains about, or contains at least 1%, 5%, 10%, 20%, 25%, 30%, 40%, 50%, 60%, or 70% or more human skim milk, e.g., w/w or w/v. In certain embodiments, the topical formulation contains no more than 75%, 70%, 60%, 50%, 40%, 30%, 25%, 20%, or 10% human skim milk. In some embodiments, the topical formulation contains between or between about 1% and 75%, 5% and 50%, or 10% and 50% human skim milk, each inclusive, e.g., w/w or w/v.

In certain embodiments, the topical formulation is formulated as a standard formulation, e.g., a formulation such as an emulsion, gel, or lotion that is typical, standard, or known in the art, where all or some of the water, or buffer solution including water such as saline or PBS, typically used to formulate topical formulation is replaced with skim milk.

Human Milk Permeate

In some embodiments, the topical formulation contains a permeate fraction of milk. In certain embodiments, the permeate results from filtration, e.g., ultrafiltration, of skim milk. In some embodiments, the permeate is or includes the portion of the skim milk that passes through a filter, e.g., an ultra-filter. In some embodiments, the permeate is enriched with milk oligosaccharides, e.g., enriched with respect to whole milk.

In certain embodiments, the topical formulation contains a permeate fraction of human milk (also referred to herein as “human milk permeate” or “permeate”). In some embodiments, the human milk permeate results from filtration, e.g., ultrafiltration, of human skim milk. In particular embodiments, the permeate is or includes the portion of the skim milk that passes through a filter, e.g., an ultra-filter. In some embodiments, the permeate is enriched with human milk oligosaccharides (HMOs), e.g., enriched with respect to whole human milk.

In some embodiments, the topical formulation contains human milk permeate. In certain embodiments, the topical formulation contains, contains about, or contains at least 1%, 5%, 10%, 20%, 25%, 30%, 40%, 50%, 60%, or 70% or more human milk permeate, e.g., w/w or w/v. In some embodiments, the topical formulation includes, contains, or is formulated with between 50% and 95%, 60% and 90%, or 60% and 80% human milk permeate, each inclusive. In particular embodiments, the topical formulation includes, contains, or is formulated with about or at least 50%, 60%, or 70% human milk permeate or between 50% and 75%, 60% and 80%, or 65% and 75%, each inclusive, e.g., w/w or w/v.

In certain embodiments, the topical formulation contains a fragment or a portion that originates from or is derived from human milk permeate. In some embodiments, the permeate or fragment or portion thereof is, includes, or is enriched for one or more HMOs. In certain embodiments, the human milk permeate, or fraction or portion thereof, is or includes one or more cofactors, vitamins, amino acids, carbohydrates, sugars, and/or nucleosides present in or native to human milk. In some embodiments, the permeate, or fraction or portion thereof, is enriched for one or more compounds present in human milk or human milk permeate. Such compounds may include but are not limited to proteins (e.g., low molecular weight proteins), cofactors, vitamins, amino acids, carbohydrates, sugars, polynucleotides, oligonucleotides (e.g., human milk oligosaccharides).

In particular embodiments, the human milk permeate, or fraction or portion thereof, is or includes a concentration of at least 0.1%, 0.2%, 0.3%, 0.4%, 0.5%, 0.75%, or 1% human milk oligosaccharides, e.g., w/w. In certain embodiments, human milk permeate that includes or contains at least 0.1%, 0.2%, 0.3%, 0.4%, 0.5%, 0.75%, or 1% human milk oligosaccharides, e.g., w/w, is added, mixed, or formulated with, or to arrive at, the topical formulation. In certain embodiments, the permeate includes or contains at least 0.5% HMO, e.g., w/w, prior to incorporation into the topical formulation.

In particular embodiments, the topical formulation contains, includes, or is formulated with a relatively small amount of human milk permeate. In certain embodiments, the topical formulation includes, contains, or is formulated at least 0.001%, 0.001%, 0.01%, 0.1%, 0.5%, 1.0%, 2.0%, 2.5%, 5.0%, 7.5%, or 10% or human milk permeate, or a fraction or portion thereof. In certain embodiments, the topical formulation contains no more than 20%, 10%, 5.0%, 2.5%, 1.0%, or 0.5% of human milk permeate or a portion or fraction thereof. In some embodiments, the topical formulation contains between or between about 0.001% and 20%, 0.01% and 5%, or 0.1% and 2% human milk permeate, each inclusive.

In certain embodiments, the topical formulation is formulated with permeate in place of water. Thus, in some embodiments, formulations such as emulsions, gels, ointments that are typically formulated with water are formulated with human milk permeate in place of water. Thus, in some embodiments, the topical formulation is or includes at least 30%, 40%, 50%, 60%, 70%, 75%, 80%, or 90% permeate.

In some embodiments, the human milk permeate, or fraction or portion thereof, is further treated or processed prior to adding, mixing, or formulating into the topical formulation. In certain embodiments, the permeate is treated to remove one or more substances, such as by filtration and/or the addition of an enzyme, e.g., lactase, to remove or digest unwanted compounds, e.g., sugars such as lactose. In some embodiments, the permeate is further treated to increase the concentration of HMOs, such as by filtration, e.g., ultra-filtration or nanofiltration, prior to adding, mixing, or formulating into the topical formulation.

In particular embodiments, the human milk permeate is processed prior to its incorporation into the topical formulation. Such processing steps may serve to enrich or increase the concentration of human milk oligosaccharides and/or to reduce the content or concentration of one or more sugars, e.g., lactose. In some embodiments, the human milk permeate is processed into a fraction or portion of human milk permeate that is or includes an increased concentration of HMOs, e.g., relative to whole human milk or unprocessed human milk permeate, and/or reduced content or concentration of one or more sugars, e.g., lactose, e.g., relative to whole human milk or unprocessed human milk permeate.

In some embodiments, permeate is processed to include less than about 10%, 9%, 8%, 7%, 6%, 5%, 4%, 3%, 2%, or 1% w/w lactose prior to its incorporation into the topical formulation. In particular embodiments, the permeate is processed to include less than 2% or 1% lactose. In certain embodiments, permeate is processed to include an HMO or total HMOs concentration at least 0.5%, 1.0%, 2.0%, 3.0% 4.0%, 5.0%, 7.5%, or 10%, 15%, or 20%, or between 1.0% and 2.0%, 2.0% and 4.0%, 4.0% and 5.0%, 1.0% and 5.0%, 5.0% and 10.0%, or 5.0% and 7.5% w/v HMO, each inclusive.

In some embodiments, the permeate has or includes an HMO profile that is substantially similar both structurally and functionally to the profile of HMOs observed across the population of whole human milk. That is to say, in some aspects, since the permeate may be obtained from a source of human milk derived from a pool of donors, rather than an individual donor, the array of HMOs will be more diverse than in any one typical individual, and will represent or more closely represent the spectrum of HMOs that are found in human milk as opposed to the spectrum of HMOs that are found or typically found in the human milk produced by any particular individual.

In some embodiments, the permeate includes a greater amount of different individual HMOs than the number of different individual HMOs found in human milk from an individual donor. In certain embodiments, the permeate includes at least 1, 2, 3, 4, 5, 10, 15, 20, 25, 30, 35, 40, 45, or 50 more individual HMOs than the number of different individual HMOs found in human milk from an individual donor. In particular embodiments, the permeate includes a greater amount of different individual HMOs than the mean or median number of different individual HMOs found in a plurality of human milk samples from individual donors.

In some aspects, one of the biggest variables in HMO diversity derives from the mother's Lewis blood group and specifically whether or not she has an active fucosyltrasferase 2 (FUT2) and/or fucosyltrasferase 3 (FUT3) gene. When there is an active FUT2 gene, an α1-2 linked fucose is produced, whereas fucose residues are α1-4 linked when the FUT3 gene is active. The result of this “secretor status” is, generally, that “secretors” (i.e. those with an active FUT2 gene) produce a much more diverse profile of HMOs dominated by α1-2 linked oligosaccharides, whereas “non-secretors” (i.e. those without an active FUT2 gene) may comprise a more varied array of, for example α1,-4 linked oligosaccharides (as compared to secretors), but comprise an overall decrease in diversity since they are unable to synthesize a major component of the secretor's HMO repertoire. In some embodiments, the human milk permeate includes human milk oligosaccharides that include α1-2 linked fucose and human milk oligosaccharides that include α1-4 linked fucose. In certain embodiments, the topical formulation includes human milk oligosaccharides that include α1-2 linked fucose and human milk oligosaccharides that include α1-4 linked fucose.

In some embodiments, the topical formulation contains one or more HMOs. In particular embodiments, the topical formulation is or includes a plurality of HMOs, e.g., a plurality of, of about, or of at least 10, 25, 50, 75, 100, or 150 HMOs. In some embodiments, the topical formulation includes some or all of 2′-fucosyl-lactose, 3-fucosyl-lactose, 3′-sialyl-lactose, 6′-sialyl-lactose, Lacto-N-tetraose, lacto-N-difucohexaose I, lactodifucotetraose, Lacto-N-fucopentaose I, sialylacto-N-tetraose c, sialylacto-N-tetraose b, and disialyllacto-N-tetraose. In particular embodiments, the topical formulation includes all of 2′-fucosyl-lactose, 3-fucosyl-lactose, 3′-sialyl-lactose, 6′-sialyl-lactose, Lacto-N-tetraose, lacto-N-difucohexaose I, lactodifucotetraose, Lacto-N-fucopentaose I, sialylacto-N-tetraose c, sialylacto-N-tetraose b, and disialyllacto-N-tetraose.

In certain embodiments, the topical formulation includes one or more of 2′-fucosyl-lactose, 3-fucosyl-lactose, 3′-sialyl-lactose, 6′-sialyl-lactose, lacto-N-tetraose, lacto-N-neo-tetraose, lacto-N-fucopentaose I, lacto-N-fucopentaose II, lacto-N-fucopentaose III, sialyl-lacto-N-tetraose a, sialyl-lacto-N-tetraose b, sialyl-lacto-N-tetraose c, lacto-N-difuco-hexaose I, lacto-N-difuco-hexaose II, lacto-N-hexaose, para-lacto-N-hexaose, disialyllacto-N-tetraose, fucosyl-lacto-N-hexaose, difucosyl-Lacto-N-hexaose a, difucosyl-Lacto-N-hexaose b, lactodifucotetraose (LD), 6′galactosyllactose, 3′galactosyllactose, 3-Sialyl-3-fucosyllactose, Sialylfucosyllacto-N-tetraose, Sialyllacto-N-fucopentaose V, disialyl-lacto-n-fucopentaose II, disialyl-lacto-n-fucopentaose V, lacto-N-neo-difucohexaose II, 3-fucosyl-sialylacto-N-tetraose c, para-lacto-N-neohexose, lacto-N-octaose, lacto-N-neooctaose, lacto-N-neohexaose, lacto-N-fucopentaose V, iso-lacto-N-octaose, para-lacto-N-octaose, lacto-decaose, and sialyl-lacto-N-fucopentaose I. In particular embodiments, the topical formulation includes all of 2′-fucosyl-lactose, 3-fucosyl-lactose, 3′-sialyl-lactose, 6′-sialyl-lactose, lacto-N-tetraose, lacto-N-neo-tetraose, lacto-N-fucopentaose I, lacto-N-fucopentaose II, lacto-N-fucopentaose III, sialyl-lacto-N-tetraose a, sialyl-lacto-N-tetraose b, sialyl-lacto-N-tetraose c, lacto-N-difuco-hexaose I, lacto-N-difuco-hexaose II, lacto-N-hexaose, para-Lacto-N-hexaose, disialyllacto-N-tetraose, fucosyl-lacto-N-hexaose, difucosyl-lacto-N-hexaose a, difucosyl-lacto-N-hexaose b, lactodifucotetraose, 6′galactosyllactose, 3′galactosyllactose, 3-sialyl-3-fucosyllactose, sialylfucosyllacto-N-tetraose, sialyllacto-N-fucopentaose V, disialyl-lacto-n-fucopentaose II, disialyl-lacto-n-fucopentaose V, lacto-N-neo-difucohexaose II, 3-Fucosyl-sialylacto-N-tetraose c, para-Lacto-N-neohexose, lacto-N-octaose, lacto-N-neooctaose, lacto-N-neohexaose, lacto-N-fucopentaose V, iso-lacto-N-octaose, para-lacto-N-octaose, lacto-decaose, and sialyl-lacto-N-fucopentaose I.

In certain embodiments, the topical formulation includes or contains at least 10, 25, 50, 100, or 150 HMOs which include all of 2′-fucosyl-lactose, 3-fucosyl-lactose, 3′-sialyl-lactose, 6′-sialyl-lactose, lacto-N-tetraose, lacto-N-difucohexaose I, lactodifucotetraose, lacto-N-fucopentaose I, sialylacto-N-tetraose c, sialylacto-N-tetraose b, and disialyllacto-N-tetraose. In particular embodiments, the topical formulation includes or contains at least 25, 50, 100, or 150 HMOs which include all of 2′-fucosyl-lactose, 3-fucosyl-lactose, 3′-sialyl-lactose, 6′-sialyl-lactose, lacto-N-tetraose, lacto-N-neo-tetraose, lacto-N-fucopentaose I, lacto-N-fucopentaose II, lacto-N-fucopentaose III, sialyl-lacto-N-tetraose a, sialyl-lacto-N-tetraose b, sialyl-lacto-N-tetraose c, lacto-N-difuco-hexaose I, lacto-N-difuco-hexaose II, lacto-N-hexaose, para-lacto-N-hexaose, disialyllacto-N-tetraose, fucosyl-lacto-N-hexaose, difucosyl-lacto-N-hexaose a, difucosyl-lacto-N-hexaose b, lactodifucotetraose (LD), 6′galactosyllactose, 3′galactosyllactose, 3-Sialyl-3-fucosyllactose, sialylfucosyllacto-N-tetraose, sialyllacto-N-fucopentaose V, disialyl-lacto-n-fucopentaose II, disialyl-lacto-n-fucopentaose V, lacto-N-neo-difucohexaose II, 3-fucosyl-sialylacto-N-tetraose c, para-lacto-N-neohexose, lacto-N-octaose, lacto-N-neooctaose, lacto-N-neohexaose, lacto-N-fucopentaose V, iso-lacto-N-octaose, para-lacto-N-octaose, lacto-decaose, and sialyl-lacto-N-fucopentaose I.

In particular embodiments, the topical formulation is or includes at least 10, 25, 50, 75, 100, 125, 150, of the different HMOs found, present, or detected in pooled human milk (e.g., pooled from the milk of at least 10, 25, 50, or 100 individual donors) or in permeate (e.g., permeate resulting from ultra-filtering human milk skim) obtained from pooled human milk. In some embodiments, the topical formulation includes at least 50%, 60%, 70%, 80%, 85%, 90%, 95%, 97%, 99%, or 99.9% of the different HMOs found, present, or detected in pooled human milk or in permeate) obtained from pooled human milk. In certain embodiments, the topical formulation includes at least 50%, 60%, 70%, 80%, 85%, 90%, 95%, 97%, 99%, or 99.9% of the individual HMOs that may be found, present, or detected across samples of human milk. In some embodiments, the topical formulation includes the same or substantially the same HMOs found, present, or detected in pooled human milk or in permeate obtained from pooled human milk.

In certain embodiments, the topical formulation contains or includes a permeate resulting from the ultrafiltration of human whole or skim milk pooled from at milk collected from at least 10, 25, 50, or 100 individual human milk donors that is further concentrated, e.g., by nanofiltration or reverse osmosis, to increase the concentration of total HMO (e.g., by w/w). In some embodiments, the concentration of total HMO is increased to at least 0.5%, 1%, 2%, 3%, 4%, 5%, 6%, 7%, 8%, 9%, or 10%. In certain embodiments, the concentration of total HMO is increased to at least 5% (w/w). In certain embodiments, the concentration of total HMO is increased to between 8% and 12% (w/w), inclusive.

In particular embodiments, the topical formulation contains one or more cofactors or vitamins found in or native to human milk. In certain embodiments, the topical formulation contains one or more cofactors or vitamins selected from threonate, pantothenante (vitamin B5), nicotinamide, N1-Methyl-2-pyridone-5-carboxamide, pyridoxal dehydroascorbate, nicotinamide ribonucleotide, and pyridoxamine. In some embodiments, the topical formulation threonate, pantothenante (vitamin B5), nicotinamide, N1-Methyl-2-pyridone-5-carboxamide, pyridoxal dehydroascorbate, nicotinamide ribonucleotide, and pyridoxamine.

In some embodiments, the topical formulation contains one or more amino acids native to or found in human milk. In certain embodiments, the topical formulation contains at least one of creatine, creatinine, or arginine. In some embodiments, the topical formulation contains creatine, creatinine, and arginine.

In some embodiments, the topical formulation contains one or more sugars, e.g., lactose, or energy sources native to or found in human milk. In certain embodiments, the topical formulation contains one or more of citrate, alpha-ketoglutarate, succinate, or phosphate. In particular embodiments, the topical formulation contains citrate, alpha-ketoglutarate, succinate, and phosphate.

Human Milk Retentate

In certain embodiments, the topical formulation contains or includes a retentate fraction of human milk or a portion or fraction thereof. In some embodiments, the retentate results from filtration, e.g., ultrafiltration, of human skim milk. In certain embodiments, the retentate, e.g., human milk retentate, is or includes the fraction of the skim milk that is retained by the filter, e.g., ultrafilter. In some embodiments, the retentate fraction, e.g., human milk retentate, contains enriched or concentrated human milk proteins, e.g., enriched or more concentrated with respect to whole human milk. In some embodiments, the retentate, e.g., human milk retentate, contains enriched or concentrated human milk proteins having a molecular weight greater than what may pass through the filter, e.g., ultrafilter. In some embodiments, the retentate fraction contains enriched or concentrated human milk proteins having a molecular weight of at least or above 1 kDa, 5 kDa, 10 kDa, 20 kDa, 25 kDa, 40 kDa, 50 kDa, 100 kDa, 250 kDa, or 500 kDa. In some embodiments, the retentate fraction contains enriched or concentrated human milk proteins that have a molecular weight of at least or above between or between about 1 kDa and 40 kDa, inclusive.

In certain embodiments, the topical formulation contains retentate, e.g., human milk retentate, or a portion or fraction thereof. In certain embodiments, the topical formulation contains, includes, or is formulated with at least 1%, 5%, 10%, 20%, 25%, 30%, 40%, 50%, 60%, or 70% or more retentate w/w or w/v, e.g., human milk retentate, or a fraction or portion thereof. In certain embodiments, the topical formulation contains no more than 30%, 25%, 20%, 10%, 5%, 1%, 0.5%, 0.1%, 0.01%, or 0.001% retentate w/w or w/v, e.g., human milk retentate, or a fraction or portion thereof. In some embodiments, the topical formulation contains between or between about 0% and 75%, 5% and 50%, or 10% and 50% retentate, e.g., human milk retentate, w/w or w/v, each inclusive.

In certain embodiments, the topical formulation contains, contains about, or contains at least 0.001%, 0.001%, 0.01%, 0.1%, 0.5%, 1.0%, 2.0%, 2.5%, 5.0%, 7.5%, or 10% or more retentate, e.g., human milk retentate, w/w or w/v. In certain embodiments, the topical formulation contains no more than 20%, 10%, 5.0%, 2.5%, 1.0%, or 0.5% retentate, e.g., human milk retentate, w/w or w/v. In some embodiments, the topical formulation contains between or between about 0.1% and 20%, 1% and 5%, or 0.1% and 2% retentate, e.g., human milk retentate, w/w or w/v, each inclusive.

In certain embodiments, the topical formulation contains or includes one or more proteins found in or native to human milk. In some aspects, human milk proteins include or may be divided into whey and casein fractions or complexes. In certain embodiments, the human milk proteins include but are not limited to casein, α-lactalbumin, lactoferrin, secretory immunoglobulin IgA, lysozyme, and serum albumin. In some embodiments, the topical formulation includes one or more human milk proteins, such as but are not limited to immunoglobulins, e.g., IgA, sIgA, IgG, and IgM; cytokines, e.g., IL-6, IL-7, IL-8, IL-10, IFN-gamma, TGF-beta, TNF-alpha; chemokines, e.g., G-CSF and MIF; growth factors, e.g., EGF, VEGF, NGF, IGF, TNFRI and TNFRII, and erythropoietin; anti-microbials, e.g., lactoferrin, lactadherin; and peptide hormones, e.g., somatostatin, calcitonin, adiponectin, leptin, and ghrelin.

Exemplary Topical Formulations

In some embodiments, the topical formulation is or includes one or more human milk fractions, portions, or components and one or more pharmaceutically acceptable excipients. In certain embodiments, the topical formulation is or includes one or more human milk fractions, portions, or components that are formulated into a pharmaceutically acceptable topical formulation, such as an oil-in water emulsion, a water-in-oil emulsion, a nanoemulsion, an emulsified gel, an oleaginous ointment, a hydrocolloid system, or a cream preparation.

In particular embodiments, the topical formulation is or includes more than one human milk fraction, portion, or component. In certain embodiments, the topical formulation is or includes human milk cream or a fraction, sub-fraction, or portion thereof. In certain embodiments, the topical formulation is or includes human milk permeate or a portion or fraction thereof. In some embodiments, the topical formulation is or includes human milk retentate or a portion or fraction thereof. In certain embodiments, the topical formulation is or includes two or more of human milk cream, skim, permeate, or retentate. In various embodiments, the topical formulation is or includes human milk cream and human milk permeate.

In particular embodiments, the topical formulation is or includes human milk cream or a sub-fraction thereof and human milk permeate. In certain embodiments, the topical formulation is or includes human milk cream and human milk permeate. In some embodiments, the topical formulation is or includes human milk butteroil and human milk permeate. In some embodiments, the topical formulation is or includes from 20% to 30% human milk fat (w/w or w/v).

In some embodiments, the topical formulation is or includes one or more human milk lipids, fats, or fatty acids. In certain embodiments, the topical formulation is or includes one or more human milk fatty acids. In certain embodiments, the topical formulation includes a plurality of human milk fatty acids. In some embodiments, the topical formulation is or includes one or more human milk oligosaccharides. In particular embodiments, the topical formulation is or includes a plurality of human milk oligosaccharides. In certain embodiments, the topical formulation is or includes one or more human milk proteins. In particular embodiments, the topical formulation includes one or more human milk fatty acids and one or more human milk oligosaccharides. In particular embodiments, the human milk formulation is or includes a plurality of human milk fatty acids and a plurality of human milk oligosaccharides.

In particular embodiments, the human milk formulation is or includes at least 1%, 2.5%, 5%, 7.5%, 10%, 15%, or 20% or between 1% and 50%, 10 and 40%, 1% and 40%, 10% and 30%, 1% and 25%, 5% and 20%, or 5% and 15% human milk lipids, fat, or fatty acids, e.g., w/v or w/w, and at least 0.01%, 0.1%, or 0.25% or between 0.1% and 1% human milk oligosaccharides, e.g., w/v or w/w, each inclusive. In certain embodiments, the topical formulation is or includes between 5% and 15% human milk lipids, fat, or fatty acids and between 0.1% and 1% human milk oligosaccharides, e.g., w/v or w/w. In particular embodiments, the topical formulation is or includes about 10% human milk lipids, fat, or fatty acids and between 0.1% and 0.5% human milk oligosaccharides, e.g., w/v or w/w. In some embodiments, the topical formulation is or includes between 15% and 30% human milk fat and between 0.1% and 1% human milk oligosaccharides, e.g., w/w. In particular embodiments, the topical formulation is or includes about 20% human milk fat and between 0.1% and 0.5% human milk oligosaccharides, e.g., w/w. In various embodiments, the topical formulation is or includes about 30% human milk fat and between 0.1% and 0.5% human milk oligosaccharides, e.g., w/w, each inclusive.

In some embodiments, the topical formulation is or includes a plurality of human milk lipids and one or more human milk oligosaccharides. In various embodiments, the topical formulation contains a plurality of human milk lipids that are or include one or more neutral complex lipids (e.g., free fatty acids, cholesteryl esters, diacylglycerols, triacylglycerols, monoacylglycersols), sphingolipids (e.g., ceramides, dihyroceramindes, hexosylceramides, lactosylceramides, sphingomyelins), phospholipids (e.g., phosphatidylcholines, lysophosphatidylcholines, phosphatidylethanolamines, lysophophatidylethanolamines, phosphatidylinositols), or a combination of any or all of the foregoing and one or HMOs.

In certain embodiments, the topical formulation is or includes a plurality of human milk fatty acids and one or more HMOs. In particular embodiments, the topical formulation contains or includes a plurality of human milk fatty acids that include some or all of oleic acid, palmitic acid, linoleic acid, omega 3 fatty acid, omega 6 fatty acid, omega 9 fatty acid, stearic acid, myristic acid, lauric acid, palmitoleic, vaccenic acid, Heptadecanoic acid, myristolic acid, heptadecenoic, docosahexaenoic, gamma linolenic, docosapentaenoic acid, eicosapentaenoic acid (EPA), arachidonic acid (AA) and docosahexaenoic acid (DHA) and one or more HMOs.

In particular embodiments, the topical formulation is or includes a plurality of human milk oligosaccharides and one or more human milk lipids, fats, fatty acids. In particular embodiments, the topical formulation is or includes a plurality of at least 10, 25, 50, or 100 human milk oligosaccharides including some or all of 2′-fucosyl-lactose, 3-fucosyl-lactose, 3′-sialyl-lactose, 6′-sialyl-lactose, Lacto-N-tetraose, lacto-N-difucohexaose I, lactodifucotetraose, Lacto-N-fucopentaose I, sialylacto-N-tetraose c, sialylacto-N-tetraose b, and disialyllacto-N-tetraose as well as one or more human milk lipids, fats, or fatty acids. In particular embodiments, the topical formulation is or includes a plurality of at least 10, 25, 50, or 100 human milk oligosaccharides including some or all of 2′-fucosyl-lactose, 3-fucosyl-lactose, 3′-sialyl-lactose, 6′-sialyl-lactose, Lacto-N-tetraose, lacto-N-difucohexaose I, lactodifucotetraose, Lacto-N-fucopentaose I, sialylacto-N-tetraose c, sialylacto-N-tetraose b, and disialyllacto-N-tetraose as well as one or more human milk lipids, fats, or fatty acids.

In various embodiments, the topical formulation is or includes a plurality of human milk oligosaccharides and a plurality of at least 10, 25, 50, or 100 human milk fatty acids. In particular embodiments, the topical formulation is or includes (i) a plurality of human milk fatty acids that include some or all of oleic acid, palmitic acid, linoleic acid, omega 3 fatty acid, omega 6 fatty acid, omega 9 fatty acid, stearic acid, myristic acid, lauric acid, palmitoleic, vaccenic acid, heptadecanoic acid, myristolic acid, heptadecenoic acid, gamma linolenic acid, docosapentaenoic acid, eicosapentaenoic acid, arachidonic acid, and docosahexaenoic acid; and (ii) a plurality of human milk oligosaccharides including some or all of 2′-fucosyl-lactose, 3-fucosyl-lactose, 3′-sialyl-lactose, 6′-sialyl-lactose, lacto-N-tetraose, lacto-N-difucohexaose I, lactodifucotetraose, lacto-N-fucopentaose I, sialylacto-N-tetraose c, sialylacto-N-tetraose b, and disialyllacto-N-tetraose.

In certain embodiments, the topical formulations are stable, e.g., may be stored for periods of time without spoilage or loss of effectivity or bioactivity. In some embodiments, the topical formulation may be stored refrigerated, e.g., at or between 2° C. and 8° C., for at least 6 weeks, 8 weeks, 10 weeks, 12 weeks, or at least 2 months, 3 months, 6 months, 9 months, or 12 months or longer, e.g., without spoilage and/or without a detectable loss of effectivity or bioactivity. In particular embodiments, the topical formulation may be stored at room temperature and/or ambient temperature, such as at or between 16° C. and 24° C. or at about 20° C., for at least 6 weeks, 8 weeks, 10 weeks, 12 weeks, or at least 2 months, 3 months, 6 months, 9 months, or 12 months or longer, e.g., without spoilage and/or without a detectable loss of effectivity or bioactivity. Particular embodiments contemplate that changes in the effectivity or bioactivity may be determined by those of skill in the art as a matter of routine, including by any of the methods or assays described herein, e.g., in the Examples. In some embodiments, stability is or includes a low degree of change in the composition of human milk components present in the topical formulation over time, such as for example the degree of change in the human milk fatty acid composition of the topical formulation. In some embodiments, a low degree of change is or includes a relative difference of less than 10%, 5%, or 1% in the concentration or amount of ten, fifteen, or twenty or more distinct human milk components, e.g., structurally distinct human milk fatty acids and/or human milk oligosaccharides, between two time points.

### II. Uses of the Provided Compostions and Formulations

Also provided herein are uses and methods for using topical formulations containing one or more portions or fractions of human milk, e.g., any one of the compositions described herein, such as in Section I. In some embodiments, the topical formulation is applied locally and/or directedly to the skin and/or a mucous membrane. In some embodiments, the topical formulation is applied locally and/or directly to skin to promote, improve, or increase hydration or barrier function. In some embodiments, the topical formulation is applied locally and/or directly to skin to improve appearance, e.g., reduce wrinkles or remove redness. In some embodiments, the topical formulation may be applied locally and/or directly to the skin to remove, remedy, or alleviate pain, irritation, or inflammation.

In particular embodiments, the topical formulation may be applied or administered to a subject to treat or prevent a disease or condition. In certain embodiments, the methods are or include administering or applying an effective amount of the provided topical formulation to a subject having or at risk of having a disease or condition, or exhibiting symptoms and/or clinical signs of a disease or condition. In certain embodiments, the disease or condition is a skin disorder or condition. In some embodiments, at least one symptom and/or clinical sign of the disease or condition, e.g., skin disorder or condition, is changed, reduced, alleviated, or ameliorated following administration or application of the topical formulation.

In certain embodiments, the skin disorder or condition is or includes acne, chronic skin ulcer, dermatitis, dermatoheliosis, infections, nipple fissure, psoriasis, rosacea, side effect of a skin cancer therapy, vitiligo, wound care, or xerosis. In some embodiments, the skin disorder or condition is or includes acne, signs of aging, cellulite, melasma, scaring, stretch marks, sun damage. In certain embodiments, the topical formulation may be applied as an anti-aging treatment, anti-cellulate treatment, cleanser treatment, face mask or body compress lip care, makeup remover, melasma reduction, moisturizer, scar reduction, skincare for pre or post-cosmetic procedures, stretch mark treatments, sun care, or toner.

In some embodiments, the skin disorder or condition is or includes dermatitis, such as but not limited to, atopic dermatitis, contact dermatitis, hand dermatitis, allergic dermatitis, gravitational dermatitis, asteatotic dermatitis, nummular dermatitis, seborrhoeic dermatitis, infective dermatitis, chronic superficial scaly acrodermatitis, chondrodermatitis and perioral dermatitis. In particular embodiments, the skin disorder or condition is acne, e.g., acne vulgaris, severe necrotizing acne, acne necrotica or necrotizing lymphocytic folliculitis, and/or varioliformis. In certain embodiments, the skin disorder or condition is rosacea, e.g., erythermatotelangietatic rosacea, papulopustular rosacea, phymatous rosacea, or ocular rosacea. In some embodiments, the skin disorder or condition is an autoimmune disorder or condition. In some embodiments, the skin condition or disorder is a drug induced skin disorder or condition. In certain embodiments, the skin disorder or condition is autoimmune or drug induced dermatomyositis, or a similar condition associated with internal pathologies such as neutrophilic dermatosis, dermatomyositis, or transient acantholytic dermatosis. In some embodiments, the skin disorders or condition is an eczema and/or is associated with inherited or metabolic conditions including but not limited to ichthyosis, xerosis or asteatosis. In some embodiments, the skin condition or disorder is a dry skin condition such as psoriasis, lupus erythematosus, exfoliative keratolysis, or any type of allergic inflammation of the skin, e.g., urticaria.

In particular embodiments, the skin condition or disorder is epidermolysis bullosa or epidermolysis bullosa simplex. In certain embodiments, the epidermolysis bullosa simplex may include, but is not limited to, epidermolysis bullosa simplex-Weber-Cockayne type, epidermolysis bullosa simplex-Koebner type, epidermolysis bullosa simplex with mottled pigmentation, epidermolysis bullosa simplex-Dowling-Meara type, or Epidermolysis bullosa simplex with muscular dystrophy.

In some embodiments, the skin condition or disorder is contagious, such as those associated with or triggered by a virus or bacteria. In some embodiments, the skin condition or disorder is or includes impetigo. In certain embodiments, the skin condition or disorder is or includes necrotizing fasciitis.

In some embodiments, the skin condition or disease is an epidermolytic or non-epidermolytic keratin-based skin disease. Exemplary types of keratin-based skin diseases include, but are not limited to, epidermolytic hyperkeratosis, ichtyosis bullosa of Siemens, pachyonychia congenita, epidermolytic or non-epidermolytic palmoplantar keratoderma (diffuse or focal) steatocystoma multiplex, Naegeli-Franceschetti-Jadassohn syndrome and dermatopathia pigmentosa reticularis.

In some embodiments, the skin condition or disorder is or results from a mechanical disruption, injury, wound, or insult. In some embodiments, the skin condition or disorder is or results from a medical or surgical treatment, such as a surgical cut or incision. In some embodiments, the skin condition or disorder is or results from a biopsy, such as but not limited to a biopsy performed with a needle or a trocar. In some embodiments, the biopsy is or includes an incisional biopsy. In certain embodiments, the biopsy is or includes a punch biopsy. In some embodiments, the skin condition or disorder is or results from a burn, such as from a radiation therapy.

In some embodiments, the skin condition or disorder is or includes contact dermatitis or contact sensitivity. In particular embodiments, the skin condition or disorder is or includes contact sensitivity to mercuric compounds (such as those found in ointments), chromate, nickel, turpentine, varnishes, resins, cosmetics, or dyes. In certain embodiments, the skin disorder or condition is or include nickel hypersensitivity. In some embodiments, the skin condition or disorder is or includes keratosis pilaris, keratodermas,

In certain embodiments, the skin disorder or condition is results from exposure to a plant, such as, for example, Toxicodendron diversilobum (poison oak), Toxicodendron radicans (poison ivy), Toxicodendron rydbergii (Rocky Mountain poison oak), Toxicodendron vernix (poison sumac or poison dogwood), Toxicodendron vernicifluum, (Japanese or Asian lacquer tree), Magnifera indica (mango tree), Anacardium occidentale (cashew tree) Gluta renghas (Rengas tree), Melanorrhoea usitata (Burmese lacquer tree), Metopium toxiferum or Comocladia dodnaea (both Caribbean shrubs), Semecarpus anacardium (India marking nut tree), or Ginkgo biloba. In some embodiments, the skin condition is or results from exposure to or bites from an arachnid or insect, e.g., a mosquito, gnat, sand fly, flea, biting fly, chigger, ant, spider, mite, or tick.

In some embodiments, the skin disorder or condition is atopic dermatitis. In certain embodiments, the pathogenesis of atopic dermatitis (and eczemas in general) involve or are associated with immunologically mediated leukocyte infiltration, such as infiltration of mononuclear cells, lymphocytes, neutrophils, and eosinophils, into the skin or dermis. In some aspects, chronic eczema also is associated with significant hyperproliferation of the epidermis. In some aspects, atopic dermatitis and eczema, if sufficiently severe, can lead to death. Less serious, but uncomfortable and often painful symptoms associated with atopic dermatitis may include but are not limited to itching, swelling, redness, blisters, crusting, ulceration, pain, scaling, cracking, hair loss, scarring, or oozing of fluid involving the skin, eye, or mucosal membranes. In particular embodiments, the topical formulation is administered or applied to the skin of a subject having atopic dermatitis. In particular embodiments, the topical formulation is administered or applied to a subject to treat, prevent, reduce, ameliorate, or alleviate atopic dermatitis. In certain embodiments, the topical formulation is administered or applied to a subject to treat, prevent, reduce, ameliorate, or alleviate one or more symptoms associated with atopic dermatitis.

In particular embodiments, the condition or disorder is psoriasis. In some aspects, psoriasis is one of the most prevalent autoimmune diseases, characterized by patches of abnormal skin. The affected skin patches are red, present scales and are itchy and irritated. In certain aspects, there are five main types of psoriasis: plaque, Guttate, inverse, pustular and erythrodermic, with plaque psoriasis is the most common. In particular embodiments, the topical formulation is administered or applied to a subject to treat, prevent, reduce, ameliorate, or alleviate psoriasis, e.g., plaque psoriasis, Guttate psoriasis, inverse psoriasis, pustular psoriasis, or erythrodermic psoriasis. In certain embodiments, the topical formulation is administered or applied to a subject to treat, prevent, reduce, ameliorate, or alleviate one or more symptoms associated with psoriasis e.g., plaque psoriasis, Guttate psoriasis, inverse psoriasis, pustular psoriasis, or erythrodermic psoriasis.

In some embodiments, the topical formulation is administered and/or applied topically. In some embodiments, the topical formulation is applied locally and/or directly to a region, e.g., a region of the skin, having or at risk of having one or more symptoms associated with a skin disorder or condition. In some embodiments, the topical formulation is administered and/or applied, e.g., directly and/or locally, to a region of the skin having or at risk of having one or more symptoms associated with a skin disorder or condition. In some embodiments, symptoms associated with a skin disorder or condition include, but are not limited to, dryness, lesions, cracks, fissures, scaling, discoloration, thickening, redness, bleeding, swelling, flaking, ulcers, sores, wounds, blistering, rashes, infection, or loss of flesh. In certain embodiments, the topical formulation is administered and/or applied to treat, prevent, reduce, ameliorate, or alleviate one or more symptoms associated with a skin disorder or condition.

In some embodiments, the topical formulation is applied to improve skin appearance, such as to reduce or prevent wrinkles or blemishes due to skin damage and/or age. In certain embodiments, application of the topical formulation to the skin may reduce inflammation, irritation, and erythema of the skin, along with an increased skin elasticity and suppleness. In certain embodiments, application of the topical formulation reduces the presence or severity of wrinkles and skin blemishes, including, but not limited to “marionette” lines, smile lines, deep nasolabial fold lines, crow's feet, fine lines/wrinkles, vertical lines between the eyebrows, horizontal forehead lines, sagging thin/frail skin, skin redness and dullness. In some aspects, improvements in barrier function is expected to improve the appearance of skin, and therefore increasing barrier function with the provided topical formulations prevent or reduce skin aging, dryness, dullness, loss of elasticity and lack of radiance. In some aspects, application or administration of the topical prevent, reduce, or slow the appearance of spider vessels or red blotchiness, exaggerated lines, and wrinkles.

In certain embodiments, the topical formulation is a moisturizer. In certain embodiments, the topical formulation is administered or applied for moisturizing skin or treating or preventing the appearing of dry skin, flaky skin, or chapped skin. In come embodiments, the topical formulations are administered or applied to treat (e.g., reduce severity, frequency, or appearance of) or to prevent skin conditions ranging from pruritus, spider veins, lentigo, age spots, senile purpura, keratosis, melasma, blotches, fine lines or wrinkles, nodules, sun damaged skin, dermatitis (including, but not limited to seborrheic dermatitis, nummular dermatitis, contact dermatitis, atopic dermatitis, exfoliative dermatitis, perioral dermatitis, and stasis dermatitis), psoriasis, folliculitis, rosacea, acne, impetigo, erysipelas, erythrasma, eczema, and other inflammatory skin conditions. In certain non-limiting aspects, the skin condition can be caused by exposure to UV light, age, irradiation, chronic sun exposure, environmental pollutants, air pollution, wind, cold, heat, chemicals, disease pathologies, smoking, or lack of nutrition. The skin can be facial skin or non-facial skin (e.g., arms, legs, hands, chest, back, feet, etc.).

In certain embodiments, the topical formulation is applied or administered to: increase the stratum corneum turnover rate of the skin; increase collagen synthesis in fibroblasts; increase cellular anti-oxidant defense mechanisms (e.g., to bolster, replenish, and/or prevent the loss of cellular antioxidants such as catalase and glutathione in skin cells (e.g., keratinocytes, melanocytes, Langerhans cells, etc.) which will reduce or prevent oxidative damage to the skin, cellular, proteins, and lipids); inhibit melanin production in melanocytes; and/or reduce or prevent oxidative damage to skin (including reducing the amount lipid peroxides and/or protein oxidation in the skin). In certain embodiments, the topical formulation is applied and/or administered to decrease the amount of internal oxidation and/or external oxidative damage in a cell. In certain embodiments, the topical formulations are administered or applied to increase collagen synthesis in a cell. In some embodiments, the topical formulation reduces skin inflammation, such as by reducing inflammatory cytokine production in a cell. Non-limiting examples of such cells include human epidermal keratinocyte, human fibroblast dermal cell, human melanocytes, three dimensional human cell-derived in vitro tissue equivalents comprising human keratinocytes, human fibroblasts, or human melanocytes, or any combination thereof (e.g., combination of human keratinocytes and human fibroblasts or a combination of human keratinocytes and human melanocytes).

In some embodiments, the topical formulation is a cosmetic formulation or composition. Non-limiting examples of cosmetic compositions or formulations include, but are not limited to, a moisturizer, a cream, a lotion, a skin softener, a foundation, a night cream, a lipstick, a cleanser, a toner, a sunscreen, a mask, or an anti-aging product.

In some embodiments, the topical formulation is or includes a cosmetic preparation that protects, moisturizes, and/or lubricates the skin. In some embodiments, the topical formulation is or includes an emollient. In certain embodiments, the topical formulation is or includes a moisturizer. In some embodiments, the topical formulation is applied or administered to the skin to perform one or more functions typically performed by sebum produced by healthy skin. In some embodiments, the topical formulation is administered or applied to the skin to prevent, reduce, or ameliorate transepidermal water loss (TEWL).

In some aspects, TEWL is normally about 4-8 g/m² h. In particular embodiments, application or administration of the topical formulation reduces or decreases the TEWL by at least 25%, 30%, 40%, 50%, 60%, 70%, 75%, 80%, or 90%, e.g., as compared to immediately before the topical formulation was applied or administered and/or as compared to untreated skin, for at least 30 minutes, 1 hour, 2 hours, 3 hours, 4 hours, 6 hours, 8 hours, 12 hours, or 24 hours.

In some embodiments, the subject is a vertebrate. In some embodiments, the subject is a mammal, such a human. In some embodiments, the subject is a human subject. In particular embodiments, the subject is an infant, a toddler, a child, an adolescent, teenager, or an adult.

Certain embodiments contemplate that the application of a topical formulation containing lipids, oligosaccharides, or proteins (or a combination thereof) derived from or native to human milk are more effective at preventing, reducing, ameliorating, or alleviating one or more symptoms in a human subject than a topical formulation containing lipids, oligosaccharides, or proteins derived from or native to a non-human source, e.g., bovine milk. In some embodiments, a “more effective” prevention, reduction, amelioration, or alleviation of one or more symptoms is or includes a greater degree of the prevention, reduction, amelioration, or alleviation of the one or more symptoms. In certain embodiments, the “more effective” prevention, reduction, amelioration, or alleviation of one or more symptoms is or includes shorter duration of time required to observe a prevention, reduction, amelioration, or alleviation following application or administration of the topical formulation. In some aspects, one of skill in the art is able to discern if a given topical formulation is more effective for the prevention, reduction, amelioration, or alleviation of the one or more symptoms as a matter of routine.

Particular embodiments contemplate that the application of a topical formulation containing lipids, oligosaccharides, or proteins (or a combination thereof) derived from or native to human milk are less likely to trigger or cause sensitization (e.g., contact dermatitis), skin irritation, inflammation, or an immune response in a human subject, e.g., on a human skin, than a topical formulation containing lipids, oligosaccharides, or proteins derived from or native to a non-human source, e.g., bovine milk. In some embodiments, the provided topical formulations are, are about, or are at least 30%, 40%, 50%, 60%, 70%, 80%, or 90% less likely to trigger or cause sensitization, skin irritation, inflammation, allergy, or an immune response in a human subject, e.g., on a human skin, than a topical formulation containing lipids, oligosaccharides, or proteins derived from or native to a non-human source, e.g., bovine milk. In some embodiments, the likelihood or probability of the topical formulation triggering or causing sensitization, skin irritation, inflammation, allergy or an immune response is less than or less than about 20%, 10%, 5%, 1%, 0.1%, or 0.01%.

In certain embodiments, the topical formulation is applied or administered to the subject to improve or increase barrier function of the skin. In certain embodiments, the subject has healthy skin. In some embodiments, the subject is experiencing one or more symptoms associated with a skin disease or condition. In particular embodiments, the topical formulation, e.g., topical formulation, improves or increases barrier function by at least 5%, 10%, 20%, 25%, 30%, 40%, 50%, 60%, 70%, 75%, 80%, 90%, 100% 150%, 200%, or 1-fold, 2-fold, 5-fold, 10-fold, 20-fold, or 50-fold, e.g., as compared to the barrier function prior to the administration or application of the topical formulation and/or as compared to untreated skin. In some aspects, the barrier function may be measured or assessed by any suitable known technique as a matter of routine, e.g., noninvasive methods that measure moisture content and loss through the skin surface such as corneometry or measurement of transepidermal water loss (TEWL).

In some embodiments, barrier function of the subject's skin may be measured or assessed by corneometry. Corneometry, in some aspects, indirectly measures barrier function by determining the capacitance of the skin e.g., a 10-20-μm thickness of the stratum corneum, due to its behavior as a dielectric medium. In some aspects, this measurement is related to the extent of hydration under various physiologic conditions in response to injury, metabolic phenomena, or topical therapies. For example, in some embodiments, changes in individual or mean skin hydration scores in a group of human subjects may serve as an index of skin health, with higher values typically considered more desirable. In some embodiments, the topical formulation, increases skin hydration as measured by corneometry by a detectable amount or by at least 5%, 10%, 20%, 25%, 30%, 40%, 50%, 60%, 70%, 75%, 80%, 90%, 100% 150%, 200%, or 1-fold, 2-fold, 5-fold, 10-fold, 20-fold, or 50-fold, e.g., as compared to the barrier function prior to the administration or application of the topical formulation and/or as compared to untreated skin.

In some embodiments, the integrity of barrier function, e.g., of the subject's skin, is assessed by measuring TEWL through the epidermal surface. In some aspects, the TEWL value is a measure of the rate of water lost through the skin (in g/h-m2) and is an estimate of the skin's ability to retain moisture. The method is based on the measure of water vapor density gradient established in a layer of 10 mm above the skin surface. It is an index of the extent of possible damage of the skin's water-barrier function. Because water loss through the skin normally occurs by passive diffusion through the epidermis, higher TEWL values indicate greater water loss and are consistent with increased damage of the barrier function of the stratum corneum such as may occur during irritant exposure or atopic dermatitis. In certain embodiments, the topical formulation increases TEWL by a detectable amount or by at least 10%, 20%, 25%, 30%, 40%, 50%, 60%, 70%, 75%, 80%, 90%, 95% 100%, e.g., as compared to the barrier function prior to the administration or application of the topical formulation.

In certain embodiments, the topical formulation, is applied or administered to the subject to treat, prevent, reduce, alleviate, or ameliorate inflammation, e.g., inflammation of the skin. In some embodiments, the inflammation is associated with a skin disorder or condition. In particular embodiments, application or administration of the topical formulation reduces the inflammation, e.g., chronic or acute inflammation. In certain embodiments, application or administration of the topical formulation reduces the amount or presence of one or more inflammatory cytokines, e.g., at or near an area where the topical formulation was administered or applied. In some embodiments, the inflammatory cytokines are or include IL-8.

In some embodiments, the topical formulation is administered or applied to a wound, e.g., a cut, scrape, lesion, laceration, or scar, such as to facilitate or improve healing or repair. In certain embodiments, the wound is healed or repaired within less time, e.g., at least 10%, 25%, or 50% less time than a similar wound that was not administered or applied the topical formulation. In particular embodiments, application of the topical formulation to a wound reduces or decreases the probability or likelihood of scarring, e.g., as opposed to similar wounds where the topical formulation is not applied.

In certain embodiments, the topical formulation is a nipple cream or a nipple balm. In certain embodiments, the topical formulation is applied to treat or prevent nipple fissure. In some embodiments, the topical formulation is applied or administered to a subject to treat or prevent soreness, skin irritation, or skin damage due to or associated with breastfeeding. In particular embodiments, a concern for nipple creams and nipple balms is that the breastfeeding infant may, e.g., inadvertently, ingest the nipple cream or nipple balm during a feeding. For example, many nipple creams or nipple balms contain lanolin, e.g., as an active ingredient. Lanolin is a by-product extract from sebum on sheep's wool after it is sheared. In addition to potential allergy risks of products derived from sheep, sheep are commonly treated with pesticides and insecticides, thereby increasing a risk exposing the infant to pesticide residues (discussed in Heikes and Craun, J. Agric. Food Chem. 1992, 40 (9), pp 1586-1590, hereby incorporated by reference in its entirety). In contrast, in some embodiments the provided topical formulations contain natural ingredients derived or originating from, or native to, human breast milk and is therefore safe for the infant and in some embodiments, may promote health benefits in the infant as well. In particular embodiments, the topical formulation is alcohol-free.

In certain embodiments, the topical formulation is administered or applied to the subject to treat, prevent, reduce, ameliorate, or alleviate stomatitis or mucositis. In certain embodiments, the topical formulation is administered or applied to the subject as an oral formulation, e.g., to be rinsed, swished, or swallowed. In some embodiments, the topical formulation is administered or applied to the subject to reduce one or more symptoms associated with stomatitis. In particular embodiments, the topical formulation is administered or applied to the subject to treat, prevent, reduce, alleviate, or ameliorate pain or inflammation anywhere in in the mouth, such as inside the cheeks, gums, tongue, lips, or palate. In some embodiments, the topical formulation is administered or applied to reduce or decrease the severity or amount of canker sores or cold sores.

In certain embodiments, the topical formulation is administered or applied to the subject to reduce one or more symptoms associated with mucositis. In particular embodiments, the topical formulation is administered or applied to the subject to treat, prevent, reduce, alleviate, or ameliorate pain, inflammation, or ulceration, anywhere along the mucous membranes lining the digestive tract. In some embodiments, the mucositis is associated with or results from a treatment for a cancer, e.g., chemotherapy, radiotherapy, or hematopoietic stem cell transplantation.

In some embodiments, the topical formulation is administered or applied to the subject to reduce one or more symptoms of a disease or condition localized to esophagus. In some embodiments, the topical formulation is administered or applied to the subject to treat, prevent, reduce, ameliorate, or alleviate one or more symptoms associated with eosinophilic esophagus. In particular embodiments, the topical formulation is administered or applied to the subject to reduce or decrease the severity or instances of swallowing difficulty, food impactation, vomiting, heartburn, or acid reflux.

### III. Manufacture of Compostions of Containing Portions or Fractions of Human Milk

In certain embodiments, the provided topical formulations are formulated, generated, or produced with one or more fractions, portions, or components of human milk. In some embodiments, one or more fractions or portions, e.g., cream, butteroil, milk fat globule membrane solids, skim milk, permeate, or retentate, of human milk are collected, isolated, or obtained from whole human milk, such as for the preparation, generation, or manufacture of the topical formulation. In certain embodiments, portions or fractions from whole human milk are obtained by methods that are similar or the same as those described in U.S. Pat. Nos. 8,545,920, 8,377,445, 9,149,052, 8,927,027, or PCT Publication Nos. WO 2014158911 and WO2018053535, hereby incorporated by reference in their entirety. In particular embodiments, one or more of the fractions, portions, or components of human milk are mixed, formulated, or incorporated into a topical formulation, e.g., a pharmaceutically acceptable topical formulation described herein.

In some embodiments, provided herein are methods of manufacturing topical formulations containing human milk or one or more portions, fractions, or components of thereof. In certain embodiments, the methods are or include isolating, separating, purifying, or obtaining a portion, fraction, or component of human milk and formulating or incorporating the portion, fraction, or component into a topical formulation such as an emulsion, cream, ointment, or lotion. In some embodiments, the portion, fraction, or component is or includes human milk cream, skim, retentate, or permeate, or a portion or fraction derived from or originating from cream, skim milk, retentate, or permeate. In some embodiments, the methods are or include formulating or incorporating two or more portions, fractions, or components of human milk (e.g., cream, skim milk, permeate, or retentate) into a topical formulation, e.g., a pharmaceutically acceptable topical formulation.

In certain embodiments, methods for manufacturing a topical formulation are or include isolating, separating, purifying, or obtaining a fraction, portion, or component from whole human milk. In certain embodiments, the fraction or portion is or includes human milk cream, butteroil, skim, retentate, or permeate (or a fragment or portion originating from or derived from cream, skim milk, retentate, or permeate). In some embodiments, the methods are or include formulating or incorporating human milk cream, skim milk, retentate, or permeate, (or a sub-fraction, fragment, or portion originating from or derived from cream such as butteroil or milk fat globule membrane solids), skim milk, retentate, or permeate) into a topical formulation such as a cream, ointment, or lotion. In particular embodiments, the isolated, separated, purified, or obtained fraction may be further processed, such as to further purify or enrich one or more components of the fraction (e.g., to purify or enrich lipids, human milk oligosaccharides, or proteins above or below a certain molecular weight) prior to formulating or incorporating into the topical formulation.

In particular embodiments, the methods for manufacturing a topical formulation are or include measuring the content, level, or concentration of one or more of macronutrients, compounds, vitamins, proteins, growth factors, fats, fatty acids, or lipids of a fraction, portion, or component from whole human milk. In some embodiments, the fraction, portion, or component of whole human milk is added to a formulation to achieve a target amount, level, or concentration of the one or more of one or more of macronutrients, compounds, vitamins, proteins, growth factors, fats, fatty acids, or lipids of a fraction, portion, or component from whole human milk. In particular aspects, the content, level, or concentration of one or more of macronutrients, compounds, vitamins, proteins, growth factors, fats, fatty acids, or lipids present in a fraction, portion, or component of whole human milk may be measured as a matter of routine by those of skill, including by any means described herein, such as in the Examples.

In particular embodiments, the manufacturing is or includes isolating, separating, purifying, or obtaining cream, skim milk, retentate, or permeate, and then further processing the cream, skim milk, retentate, or permeate, to enrich or purify one or more fractions, portions, or substances within the cream, skim milk, retentate, or permeate prior to incorporation or formulation into the topical formulation. In certain embodiments, human milk cream is processed to generate sub-fractions, e.g., butteroil and/or milk fat globule membrane solids.

In certain embodiments, provided herein are topical formulations that contain one or more fractions of human milk. In particular aspects, the topical formulation contains a fraction or a portion of human milk that is obtained or derived from a whole human milk source. In some embodiments, the whole human milk source is whole human milk obtained from one or more individual human donors. In certain embodiments, the source is whole human milk pooled from multiple individual donors.

In certain embodiments, the methods of manufacturing are or include isolating, separating, purifying, or obtaining one or more fractions from human milk, such as from a whole human milk source, e.g., whole human milk from an individual donor or pooled from multiple donors. In particular embodiments, samples are collected from the human milk source, e.g., pooled human milk, to test for contamination. Tests for contamination may include, but are not limited to, PCR tests for viruses such as HIV, HBV, and HCV.

In some embodiments, the human milk or the portions, fractions, or components thereof are processed to reduce bioburden. In some embodiments, the process to reduce bioburden may include one or both of filtration and pasteurization (or any other suitable known method of reducing bioburden). In certain embodiments, the human milk or fractions or portions thereof may be processed for reducing bioburden, e.g., filtered or pasteurized, at any stage of the process for manufacturing topical formulations. In some embodiments, human milk or fractions or portions thereof may undergo more than one process for reducing bioburden. In particular embodiments, the human milk or fractions or portions thereof undergo a process for reducing bioburden more than once, e.g., a first filtration and/or pasteurization of whole human milk and a second filtration and/or pasteurization of a fraction or portion of human milk subsequently purified, separated, or removed from the whole human milk.

In some embodiments, the temperature of the human milk and any fractions or portions thereof is continuously monitored during the process for manufacturing topical formulation, for example to insure consistency between each batch and to verify that the human milk or its fractions or portions thereof do not reach temperatures outside of threshold temperatures that may exist at each phase of the process.

In some embodiments, human milk from multiple individual donors is pooled and filtered through a large micron filter or screen, such as to remove clumps or large particulate matter. In some embodiments, the pooled whole human milk is filtered through a filter or a screen. In certain embodiments, the filter or screen contains a pore size, e.g., a mean or median pore size, of between 100 μm and 1,000 μm. In particular embodiments, the screen contains a pore size, e.g., a mean or median pore size, of at least or about 100 μm, 200 μm, 250 μm, 500 μm, or 1,000 μm. In certain embodiments, the whole human milk is filtered through a screen with a median or mean pore size of or of about 200 μm.

In certain embodiments, the process to reduce bioburden is or includes heat treatment, e.g., pasteurization. In some embodiments, the whole human milk is pasteurized or heat treated by any suitable means, including but not limited to techniques such as vat or batch pasteurization, high-temperature short-time (HTST), higher heat shorter time (HHST), ultra-pasteurization (UP), or ultrahigh temperature (UHT). In some embodiments, portions, or fractions of milk with high fat or lipid contents, e.g., greater than 10%, are pasteurized at a higher temperature than whole milk or lower fat fractions, e.g., at least 3° C. higher.

In some embodiments, human milk pooled from individual donors are separated into one or more fractions, e.g., cream, permeate, and retentate. In some embodiments, the fractions are separated by a process that includes the steps of (a) filtering the pooled, whole human milk through a 200 μm filter, (b) pasteurizing the milk at a temperature of or of about 63° C. for or for about 30 minutes, (c) generating a cream fraction and a skim milk fraction from the whole human milk by centrifugation, and (d) ultra-filtering some or all of the skim milk is to generate permeate and retentate fractions. In some embodiments, the one or more of the human milk cream, skim, permeate, or retentate fractions are formulated into the topical formulation. In some embodiments, the cream, skim, permeate, or retentate fractions are further processed such as to enrich one or more compounds, e.g., by filtration or centrifugation, prior to incorporation into a topical formulation.

In particular embodiments, the methods of manufacturing the topical formulation are or include isolating, separating, purifying, or obtaining cream (or a fragment or portion originating from or derived from cream) and one or more of retentate or permeate (or portions or fractions derived from or originating from retentate or permeate) from whole human milk for incorporation into the topical formulation. In some embodiments, the methods of manufacturing the composition are or include isolating, separating, purifying, or obtaining cream (or a fragment or portion originating from or derived from cream) and permeate (or portions or fractions derived from or originating from the permeate) for incorporation into the topical formulation.

In various embodiments, the methods of manufacturing a composition are or include isolating, separating, purifying, or obtaining cream and permeate (or portions or fractions derived from or originating from cream and/or permeate) from whole human milk and formulating or incorporating the cream and the permeate (or the fragments or portions originating from or derived from the cream and/or permeate) into the composition, e.g., the topical formulation. In some embodiments, the methods of manufacturing the composition are or include formulating or incorporating cream (or a sub-fragment or portion originating from or derived from cream such as butteroil or milk fat globule membrane solids) and one or more of the retentate or permeate (or portions or fractions derived from or originating from the permeate) that was isolated, separated, purified, or obtained from whole human milk into the composition, e.g., the topical formulation.

Cream and Skim Fractions

In some embodiments, the cream and skim milk fractions are obtained by removing the fat or cream from whole human milk. In some embodiments, the whole human milk is separated into skim milk and cream fractions, such as by known or routine methods, including but not limited to centrifugation. In some embodiments, the whole human milk is centrifuged to separate cream and skim milk fractions. In particular embodiments, the topical formulation contains cream obtained by centrifuging whole human milk. In certain embodiments, the compositions, e.g., topical formulation, contain skim milk obtained by removing cream by centrifugation or creaming separation.

Particular embodiments contemplate that methods and techniques for the removal of cream from whole milk, e.g., whole human milk or breast milk, are known and can be performed as a matter of routine. In some embodiments, the methods of manufacturing a composition are or include isolating, separating, purifying, or obtaining cream (or a fragment or portion originating from or derived from cream such as butteroil or milk fat globule membrane solids sub-fraction) from whole human milk and formulating or incorporating the cream, or the sub-fraction thereof, into the composition, e.g., the topical formulation. In some embodiments, the methods of manufacturing the composition are or include formulating or incorporating cream or the sub-fraction thereof, that was isolated, separated, purified, or obtained from whole human milk into the topical formulation.

In certain embodiments, the cream, e.g., human milk cream, is obtained from a method or technique that is or includes creaming separation or centrifugation. In some aspects, creaming separation allows for the combination of fat globules over time thus allowing for the accumulation of fat globules in a cream layer towards the surface of the milk. In some aspects, whole milk may be heated, e.g., to between 20° C. and 50° C. (68° F. and 122° F.), to increase the speed of the fat globule combinations and accumulation in a cream layer. In particular embodiments, the topical formulation is or includes cream, e.g., human milk cream, obtained or collected by creaming separation. In some embodiments, the cream is obtained by centrifugation of whole milk. In certain aspects, centrifugation relies on spinning whole milk in a centrifuge, e.g., at between 5,000 and 10,000 RCF, to produce cream and skim layers. In certain aspects, the cream fraction may be further centrifuged, e.g., such as to enrich or concentrate the lipid, fat, or fatty acid content. In certain embodiments, the topical formulation contains cream, e.g., human milk cream, that is separated or obtained by centrifugation.

In some embodiments, the human milk cream is homogenized prior to formulation into the topical formulation. In some aspects, the homogenization may be performed by any suitable known means as a matter of routine, including but not limited to mechanical homogenization, pressure homogenization, or ultrasonic homogenization. In certain embodiments, homogenization of the human milk cream results in a mean fat globule size of about or under 3 μm, 2 μm, 1 μm, 0.5 μm, or 0.1 μm in diameter. In particular embodiments, homogenization of the human milk cream results in a mean fat globule size of about or under 0.5 μm in diameter. In some embodiments, about or at least 60%, 70%, 75%, 80%, or 95% of the milk fat globules of the homogenized cream have a diameter from 0.2 μm to 2 μm.

In some embodiments, the methods include measuring the fat, lipid, or fatty acid content of the human milk cream. In certain embodiments, the cream fraction may be further processed, such as by centrifugation, to achieve a target fat, lipid, or fatty acid content. In some embodiments, the human milk cream initially contains between 20% and 60% fat, lipid, of fatty acid content, and is further processed, e.g., centrifuged, to increase the fat, lipid, or fatty acid content to a target concentration, e.g., 50% to 60% or at least 70%, 80%, 90%, 95%, or more fat, lipid, or fatty acid content (such as by w/w or w/v).

In certain embodiments, the manufacture includes one or more steps for isolating, separating, or obtaining a fraction (also referred to herein as a sub-fraction), portion, or component of the cream. For example, in some embodiments, the human milk cream is further processed, e.g., centrifuged, to remove additional or residual skim milk present in the cream. In particular embodiments, the human milk cream is centrifuged to produce a fraction or portion of the cream that is or contains enriched or concentrated human milk lipids, e.g., as compared to an untreated cream. In some embodiments, the centrifugation of the cream produces or results in a human milk butter or butteroil. In some embodiments, the human milk butter or butteroil is incorporated into a composition, e.g., topical formulation. In certain embodiments, human butter is or includes at least 60%, 65%, 70%, 75%, 80%, 85%, 90%, 95%, 99% or more human milk lipids or human milk fats.

In some embodiments, one or more lipids or subsets of lipids are purified, isolated, separated, or obtained from human milk cream, butteroil, or butter. In certain embodiments, at least one of, a plurality of, or all or substantially all of the neutral complex lipids (e.g., free fatty acids, cholesteryl esters, diacylglycerols, triacylglycerols, monoacylglycersols), sphingolipids (e.g., ceramides, dihyroceramindes, hexosylceramides, lactosylceramides, sphingomyelins), phospholipids (e.g., phosphatidylcholines, lysophosphatidylcholines, phosphatidylethanolamines, lysophophatidylethanolamines, phosphatidylinositols), or a combination any or all of the foregoing, are purified, isolated, separated, or obtained from human milk cream, milk fat, butter, or butteroil and are formulated into the topical formulation.

In some embodiments, one or more lipids found in or native to human milk are synthesized from, isolated from, or purified from a non-human milk source and are formulated into a composition, e.g., a topical formulation. In some embodiments, non-human milk sources may include, but are not limited to, milk from non-human mammals, e.g., bovine, goat, or camel, or lipids found in or produced by plants. In come embodiments, one or more lipids found in human milk are chemically synthesized from chemical or lipid precursors and are formulated into the topical formulation.

In some embodiments, one or more fatty acids are purified, isolated, separated, or obtained from human milk cream, butter, or butteroil. In certain embodiments, at least one of, a plurality of, or all or substantially all of oleic acid, palmitic acid, linoleic acid, omega 3 fatty acid, omega 6 fatty acid, omega 9 fatty acid, stearic acid, myristic acid, lauric acid, palmitoleic, vaccenic acid, Heptadecanoic acid, myristolic acid, heptadecenoic, docosahexaenoic, gamma linolenic, docosapentaenoic acid, eicosapentaenoic acid (EPA), arachidonic acid (AA) and docosahexaenoic acid (DHA), or a combination any or all of the foregoing, are purified, isolated, separated, or obtained from human milk cream or butter and are formulated into the topical formulation.

In some embodiments, one or more fatty acids found in or native to human milk are synthesized from, isolated from, or purified from a non-human milk source and are formulated into a composition, e.g., a topical formulation. In some embodiments, non-human milk sources may include, but are not limited to, milk from non-human mammals, e.g., bovine, goat, or camel, or lipids found in or produced by plants. In come embodiments, one or more lipids found in human milk are chemically synthesized from chemical or lipid precursors and are formulated into a topical formulation.

Cream Sub-Fractions

In certain embodiments, the human milk cream is separated into one or more sub-fractions. In certain embodiments, the human milk cream sub-fractions are obtained by removing water and/or nonfat solids. In certain embodiments, sub-fractions of the human milk cream are generated or produced by a process that is or includes centrifuging the human milk cream. In particular embodiments, the human milk cream is homogenized prior to the centrifugation. In some aspects, homogenization may be performed as a matter of routine, including but not limited to mechanical homogenization, pressure homogenization, or ultrasonic homogenization.

In certain embodiments, the human milk cream is homogenized prior to centrifugation, resulting in a mean fat globule size of about or under 3 μm, 2 μm, 1 μm, 0.5 μm, or 0.1 μm in diameter. In some embodiments, the human milk cream is homogenized to result in a mean fat globule size of about or under 0.5 μm in diameter. In some embodiments, about or at least 60%, 70%, 75%, 80%, or 95% of the milk fat globules of the homogenized cream have a diameter from 0.2 μm to 2 μm.

In some embodiments, the human milk cream is pressure homogenized and the homogenized human milk cream is centrifuged to generate the human milk cream sub-fractions. In some embodiments, the human milk cream sub-fractions are produced or generated by pressure homogenizing the human milk cream at a pound or pound-force per square inch (psi) of about or at least 1,000 psi, 2,500 psi, 5,000 psi, 10,000 psi, 15,000 psi, 20,000 psi, 25,000 psi, 30,000 psi, 40,000 psi, or 50,000 psi at a rate of at least 1 mL/min, 10 mL/min, 25 mL/min, 50 mL/min, 60 mL/min, 70 mL/min, 80 mL/min, 90 mL/min, 100 mL/min, 150 mL/min, 200 mL/min, 300 mL/min, 400 mL/min, 500 mL/min, or 1,000 mL/min, and then centrifuging the homogenized human milk cream. In certain embodiments, the human milk cream may be pressure homogenized for one cycle or circulation. In particular embodiments, the human milk cream may be pressure homogenized for multiple cycles or recirculations, e.g., pressure homogenized for at least two, three, four, five, or ten recirculations.

In particular embodiments, the human milk cream sub-fractions are produced or generated by pressure homogenizing the human milk cream at 10,000 psi to 25,000 psi at a rate of at least 10 mL/min to 100 mL/min for at least one cycle or circulation and then centrifuging the homogenized human milk cream. In particular embodiments, the human milk cream is pressure homogenized at a pressure of at least or about 20,000 psi at a rate of at least or about 60 mL/min for at least one cycle or circulation.

In certain embodiments, the human milk cream sub-fractions are produced or generated by homogenizing the human milk cream and then centrifuging the homogenized human milk cream at about or at least 100 g, 500 g, 1,000 g, 1,500 g, 2,000 g, 2,500 g, 3,000 g, 4,000 g, 5,000 g, or 10,000 g for about or for at least 1, 5, 10, 15, 30, 45, 60, 75, or 90 minutes or for about or for at least 1, 2, 3, 6, 12, 18, or 24 hours. In certain embodiments, the homogenized human milk cream is centrifuged at 3500 g for 30 minutes, or at an alternative protocol to achieve equivalent sedimentation. Particular embodiments contemplate that one of skill may calculate alternative centrifugation settings or protocols to achieve the same or similar sedimentation results as those resulting from a given g force and time.

In certain embodiments, the human milk cream, e.g., homogenized human milk cream, is centrifuged, e.g., at 3500 g for 30 minutes or equivalent protocol, resulting in separate layers. In certain embodiments, the top layer following centrifugation of the human milk cream is a butteroil sub-fraction of the human milk cream. In some embodiments, the human milk cream is centrifuged into three layers that include a top layer that is or includes butteroil, a middle layer that is or includes human milk fat globule membrane solids, and a bottom layer that is or includes human skim milk. In certain embodiments, the butteroil fraction contains at least 80%, 90%, 95%, 99%, or 99.8% human milk fatty acids by weight/weight. In certain embodiments, the relative centrifugal force or g-force and/or the time of the centrifugation may be increased, such as to increase the fatty acid content and reduce the water or skim milk content of the butteroil layer.

In some embodiments, butteroil is obtained or derived from human milk. In some embodiments, the butteroil is obtained or derived from human milk by an Alfa-Laval process. In certain embodiments, the Alfa-Laval process is or includes a step of heating or warming human milk cream, e.g., human milk cream having 30-40% fat or fatty acid content, such as to a temperature of 55-58° C. and then passing the warmed or heated human milk cream through a pre-concentrator, such as a hermetic separator, to concentrate the fat or fatty acid concentration to about 70% to about 75% or higher. In some embodiments, the resulting concentrated cream composition is then homogenized, e.g., with a centrifixator, and then passed through a paring chamber, e.g., for phase inversion into a water-in-oil emulsion. In certain embodiments, fat is separated from the water-in-oil emulsion, e.g., such as by passing the oil and water emulsion through a concentrator to separate out the fat and/or the fatty acids. In some embodiments, remaining residual water may be removed through the use of a vacuum chamber. In some embodiments, the butteroil is obtained or derived from human milk by a Westfalia process. In certain embodiments, the Westfalia process is or includes a step for removing nonfat milk solids from human milk cream, e.g., such as by placing the human milk cream into a concentrator that removes the nonfat-milk solids portion. In particular embodiments, the Westfalia process also includes a step for homogenization, such as to break the membrane around the fat globules, and then centrifugation to remove the aqueous phase in the centrifuge.

Permeate Fractions

In some embodiments, the human skim milk is filtered, thereby producing or resulting in a retentate fraction and a permeate fraction, e.g., human milk retentate and human milk permeate fractions. In certain embodiments, the human skim milk undergoes filtration, e.g., ultrafiltration, such as with a membrane or filter having a pore size capable of filtering substances with a molecular weight of between 1-10,000 kDa. In some embodiments, the skim milk is filtered to obtain the human milk retentate and the human milk permeate. Details of this process can be found, for example, in U.S. Pat. Nos. 8,545,920; 7,914,822; 7,943,315; 8,278,046; 8,628,921; and 9,149,052; and in PCT Publication WO 2018/053535, each of which is hereby incorporated by reference in its entirety.

In particular embodiments, the retentate, e.g., human milk retentate, is the fraction that is captured during or by the ultra-filtering of the human skim milk. In certain embodiments, the retentate contains concentrated or enriched milk proteins, e.g., having a high molecular weight with respect to filter. In certain embodiments, the permeate fraction is or is obtained from the liquid that passes through the filter during the filtration of the human skim milk. In certain embodiments, the permeate contains human milk oligosaccharides. In some embodiments, the human milk oligosaccharides of the permeate fraction are concentrated or enriched with respect to the skim or retentate fractions.

In some embodiments, the human skim milk is filtered with an ultrafiltration membrane(s). In particular embodiments, the membrane is sized to prevent the passage of any substance with a molecular weight greater than or greater than about 0.1 kDa, 1 kDa, 5 kDa, 10 kDa, 20 kDa, 25 kDa, 30 kDa, 40 kDa, or 50 kDa. In certain embodiments, the membrane is sized to prevent the passage of substances having a molecular weight of over 40 kDa. In particular embodiments, the membrane prevents or captures substances having a molecular weight greater than a molecular weight within the range of between 1 kDa and 40 kDa. In some embodiments, the filter is or includes an ultra-filter. In particular embodiments, the filter is or includes a 10 kDa filter. In certain embodiments, the filter is or includes a 25 kDa filter. In some embodiments, the filter is or includes a 40 kDa filter. In some embodiments, the membrane has a mean or median pore size of less than 5 μm, 1 μm, 0.5 μm, 0.25 μm, 0.2 μm, 0.1 μm, or smaller. In particular embodiments, the filter is a 0.45 μm filter. In particular embodiments, the filter is a 0.3 μm filter. In certain embodiments, the filter is a 0.2 μm filter. In some embodiments graded filtration can be used (e.g., a first filtration at 0.45 μm and a second at 0.3 μm and a third at 0.2 μm, or any combination thereof.)

Other suitable membranes may include ultrafiltration membranes having a 3.5 kDa or less molecular weight cut-off, for example, from Dow Denmark, Naskov, Denmark. Ultrafiltration membranes made of ceramic materials may also be used. Ceramic filters have an advantage over synthetic filters as they may be sterilized with live steam.

In some embodiments, the methods include measuring the human milk oligosaccharide content of the human milk permeate. Suitable methods for measuring human milk oligosaccharide content are known and include, but are not limited to, liquid chromatography-mass spectrometry (LC-MS) techniques. In certain embodiments, the permeate fraction may be further processed, such as filtration e.g., nanofiltration, to achieve a target human milk oligosaccharide content. In some embodiments, the human milk permeate initially contains between or between about 0.1% and 1.0% human milk oligosaccharide content, e.g., by w/w or w/v. In some embodiments, the human milk permeate is processed into an HMO composition as described herein, such as to enrich HMO content and/or reduce the content of one or more sugars, e.g., lactose.

In some embodiments, the permeate fraction may be filtered a second time, such as to produce a second retentate fraction. In certain embodiments, the additional filtration further removes proteins and residual lipids or other macromolecules from the permeate. In certain embodiments, the second retentate fraction is added or pooled to the first retentate fraction, such as to increase biological agent concentration (e.g. protein concentration).

In certain embodiments, the permeate fraction is measured for HMO content prior to steps for formulation with one or more excipients. In certain embodiments, permeate is assessed to confirm that the HMO content is above a minimum concentration prior to adding to one or excipients. In certain embodiments, only permeate fractions that contain at least 0.01%, 0.05%, 0.1%, 0.5%, or 1% HMO (w/w) are formulated with one or more excipients to manufacture of topical formulation. In some embodiments, only permeate fractions that contain at least 0.1% HMO (w/w) are formulated with one or more excipients to manufacture of topical formulation. In certain embodiments, only permeate fractions that contain at least 0.5% HMO (w/w) are formulated with one or more excipients to manufacture of topical formulation. In particular embodiments, only permeate fractions that contain at least 1.0% HMO (w/w) are formulated with one or more excipients to manufacture of topical formulation.

In some embodiments, the permeate is further or additionally processed prior to mixing, formulating, or adding to one or more excipients. In particular embodiments, the permeate is further processed to enrich or purify HMOs, e.g., as compared to whole human milk, human skim milk, or the permeate fraction prior to the additional processing steps. In some embodiments, the permeate further or additional processing reduces one or more components of the permeate, e.g., lactose or minerals. In certain embodiments, the permeate is further or additionally processed to reduce, e.g., substantially reduce, levels of lactose, e.g., as compared to whole human milk, human skim milk, or the permeate fraction prior to the additional processing steps.

In particular embodiments, suitable additional or further steps for permeate are described in PCT publication No. WO 2018053535 (incorporated herewith in its entirety). In some embodiments, the further or additional processing steps are or include biochemical and/or enzymatic reduction or removal of lactose from the permeate fraction, such as without or with minimal loss of yield or change in molecular profile of the HMO content of the human permeate. In certain embodiments, the permeate is processed to reduce or remove lactose and/or to enrich or increase HMO concentration prior to its incorporation into a topical formulation.

In particular embodiments, the permeate may be processed to further enrich or purify HMOs, e.g., as compared to whole milk, skim milk, or unprocessed permeate. In certain embodiments, such processing may include additional filtration or chromatography techniques.

In some embodiments, the permeate undergoes one or more steps for concentrating or increasing the HMO content. In some embodiments, the permeate undergoes filtration, such as with nanofiltration or reverse osmosis, to concentrate or increase HMO content. In some embodiments, the nanofiltration or reverse osmosis is performed with a nano-filter, e.g., a filter between having a pore size of from or from about 1 to 10 nanometers. In particular embodiments, the filter is from or from about 100 Da to 1,000 Da in size. In certain embodiments, the filter is from or from about 400 Da to 600 Da in size. In some embodiments, the filter is from or from about 400 Da to 500 Da in size. In certain embodiments, the filter is or is about 400 kDa. In certain embodiments, the filter is or is about 500 kDa. In certain embodiments, the permeate is passed through one or more filters, e.g., by nanofiltration or reverse osmosis, that collect or prevent flow through of human milk oligosaccharides. In some such embodiments, material that does not pass through the filter is enriched for human milk oligosaccharides, and is collected as the processed permeate for use the formulation or manufacture of a topical formulation.

In certain embodiments, the HMO content of the permeate is concentrated or increased to contain at least 0.5%, 1%, 2%, 3%, 4%, 5%, 6%, 7%, 8%, 9%, or 10% HMO (w/w or w/v). In some embodiments, the HMO content of the permeate is concentrated to between 1% and 10%, 2% and 12%, or 4% and 8%. In particular embodiments, the HMO concentration of the permeate is concentrated to at least 5%.

In some embodiments, the permeate undergoes one or more processing steps to remove or reduce lactose content. In some embodiments, a lactase enzyme is contacted or added to the permeate, and the resulting permeate/lactase mixture is incubated under conditions that allows for enzymatic digestion of lactate. In some embodiments, such conditions may include but are not limited to pH, lactase concentration, temperature, mixing conditions, and duration of time. In certain embodiments, the permeate/lactase mixture is incubated at a pH of between or between about 4.3 to 4.7, e.g., at a pH of 4.5 at a temperature of between or between about 48° C. and 55° C., e.g., at or at about 50° C. In some embodiments, the permeate/lactase mixture contains between or between about 0.01% and 1.0% lactase (w/w or w/v), such as 0.1% lactase. In certain embodiments, the mixture may be incubated for at least 5, 15, 30, 60, 90, 120, or 180 minutes. In some embodiments, the mixture is incubated for or for about 60 minutes. After the incubation, in some embodiments, the mixture may be cooled, such as to between or between about 20° C. to about 30° C., e.g., to or to about 25° C.

In some embodiments, the lactase is removed from the mixture via filtration. In some embodiments, the lactase is removed from the mixture with a filter having a pore size that collects or prevents flow through of substances having a molecular weight above 50 kDa. In some such embodiments, material that flows through the filter is collected as the processed permeate to be formulated with one or more excipients into a topical formulation. In particular embodiments, the filtration removes at least 50%, 75%, 90%, 95%, 99%, 99.9%, or 99.99% of the lactase from the mixture.

In certain embodiments, the processed permeate is further filtered through one or more additional filters, such as additional filters having a pore size of or of about 2,000 to about 3,000 Daltons. In some embodiments, the one or more additional filters comprises a membrane with a pore size of <600 Daltons. In some embodiments, the composition is filtered through both a filter comprising a membrane of about 2,000 to about 3,000 Daltons followed by filtration through a membrane of <600 Daltons.

In some embodiments, the permeate fraction or portion thereof is or includes a purified HMO composition, e.g., an HMO composition having enriched or purified HMOs as compared to whole human milk or human permeate, e.g., that has not been additionally processed or filtered. In some embodiments, the purified HMO composition has a reduced level of lactose and minerals compared to whole human milk or human permeate e.g., that has not been additionally processed or filtered.

In certain embodiments, the permeate or fraction or portion thereof, e.g., the HMO composition, is stored below 37° C., below 25° C., below 10° C., or between or between about 2° C. and 8° C., such as at or at about 4° C. In some embodiments, the permeate or fraction or portion thereof is frozen and stored. In some embodiments, the permeate or fraction or portion thereof is stored until subsequent use, e.g., incorporation into a topical formulation.

In some embodiments, the pH of the human milk permeate or fraction or portion thereof is adjusted prior to incorporation into the topical formulation. In certain embodiments, the pH of the human milk permeate or the fraction or portion thereof is adjusted to an acid pH. In particular embodiments, the pH of the human milk permeate or the fraction or portion thereof is adjusted to a pH of between 5.0 and 7.0, 5.0 and 6.0, or 5.3 and 5.7. In certain embodiments, the pH of the human milk permeate is adjusted to or to about 5.2. In some embodiments, the pH of the human milk permeate is adjusted to or to about 5.5.

Retentate Fractions

In particular embodiments, the retentate fraction is or contains proteins and compounds having a molecular weight that is above a value between or between about 1 kDa and 10,000 kDa such as between or between about 1 kDa and 200 kDa. In certain embodiments, the retentate fraction is enriched for proteins and compounds having a molecular weight that is above a value between or between about 1 kDa and 10,000 kDa such as between or between about 1 kDa and 200 kDa, e.g., as compared to skim or permeate fractions In some embodiments, the retentate includes human milk substances having a molecular weight of, of about, or of greater than 0.5 kDa, 1 kDa, 2 kDa, 3 kDa, 4 kDa, 5 kDa, 10 kDa, 15 kDa, 20 kDa, 25 kDa, 30 kDa, 40 kDa, 50 kDa, 60 kDa, 70 kDa, 75 kDa, or 100 kDa. In some embodiments, the retentate includes human milk substances, e.g., proteins, that have a molecular weight that is above or above a value that is less than 10 kDa, 5 kDa, 3.5 kDa, 2.5 kDa, or 1 kDa. In particular embodiments, the retentate includes human milk substances, e.g., proteins, having a molecular weight of, of about, or of at least 40 kDa. In some embodiments, the retentate includes human milk substances, e.g., proteins, having a molecular a value that is less than 3.5 kDa.

In some embodiments, human milk proteins that are collected or trapped by the ultrafilter include one or more of (with approximate molecular weights noted in parentheses) lactalbumin (14 kDa); casein (23 kDa); lactoglobulin (37 kDa); albumin (65 kDa); and immunoglobulins (>100 kDa). In some embodiments, the retentate includes or contains one or more of human lactalbumin, human casein, human lactoglobulin, human albumin, and human immunoglobulins. In particular embodiments, the human retentate is or includes enriched or purified human milk proteins, e.g., human lactalbumin, human casein, human lactoglobulin, human albumin, or human immunoglobulins, as compared to whole human milk.

In particular embodiments, the retentate, e.g., human milk retentate fraction is stored below 37° C., below 25° C., below 10° C., or between or between about 2° C. and 8° C., such as at or at about 4° C. In some embodiments, the human retentate is frozen and stored. In certain aspects, the human retentate is stored until subsequent use, e.g., incorporation into a topical formulation, or further fractionation to produce permeate and retentate fractions.

Exemplary Methods

In some embodiments, the methods of manufacturing the topical formulation are or include isolating, separating, purifying, or obtaining one or more fractions, portions, or components from human milk, e.g., whole human milk pooled from the milk of multiple individual donors. In some embodiments, the whole milk is processed, such as by centrifugation or creaming separation, to collect, separate, or derive human milk cream and human skim milk. In some embodiments, the lipid, fat, or fatty acid content of the cream is measured, and optionally further centrifuged such as to increase the lipid, fat, or fatty acid content to a target value or range of values of lipid, fat, or fatty acid content, e.g., between 50% and 60% w/v or w/w. In particular embodiments, the fat content of the cream is measured, and optionally further centrifuged such as to increase the fat content to a target value or range, e.g., at least 60%, 70%, 80%, 90% w/w or more. In certain embodiments, the skim milk may optionally be further centrifuged to further remove fat, lipid, or fatty acid content, such as to generate a skim having less than 1%, 0.9%, 0.8%, 0.7%, 0.6%, 0.5%, 0.4%, 0.3%, 0.2%, or 0.1% total fat, lipid, or fatty acid content, e.g., w/w or w/v.

In some embodiments, the methods of manufacturing the topical formulation are or include isolating, separating, purifying, or obtaining one or more fractions from whole human milk pooled from the milk of multiple individual donors. In some embodiments, the human milk is pooled from the milk of at least 10, 20, 30, 40, 50, 75, or 100 individual donors. In certain embodiments, the human milk is pooled from the milk of from 50 to 200 donors.

In certain embodiments, the human skim milk is filtered, e.g., ultra-filtered, to produce human milk permeate and human milk retentate fractions. In certain embodiments, the oligosaccharide, e.g., HMO, content of the human milk permeate may be measured, such as to ensure or confirm that the permeate contains a threshold HMO content, e.g., at least 0.1%, 0.5%, 1%, or 5% HMO w/w or w/v, and in some instances, may be optionally processed, e.g., by an additional filtration step, to increase the HMO concentration to achieve a target value or range of HMO content, e.g., at least 0.5%, 1%, or 5% HMO w/w or w/v.

In particular embodiments, the protein content of the human milk retentate may be measured, such as to ensure or confirm that the retentate contains a threshold protein content, e.g., at least 1%, 3%, 5%, 6%, 7%, 8%, 9% or 10% or between 5% and 8% or 6% to 7% total protein content, e.g., w/w or w/v, and in some instances, the human milk retentate may be optionally processed, e.g., by an additional filtration step, to increase the protein concentration to achieve a target value or range of protein content, e.g., at least 6%, 7%, 8%, 9% or 10% total protein content by w/w or w/v.

In some embodiments, whole human milk or one or more human milk fractions, portions, or components thereof are added to, mixed with, or combined with one or more pharmaceutically acceptable excipients to create, formulate, or produce a topical formulation. In some embodiments, the whole human milk or one or more human milk fractions, portions, or components thereof are formulated into a topical formulation that is an oil-in water emulsion, a water-in-oil emulsion, a nanoemulsion, an emulsified gel, an oleaginous ointment, a hydrocolloid system, a cream preparation, or any other pharmaceutically acceptable formulation suitable for use as a topical formulation.

In some embodiments, human milk cream is added or incorporated into the topical formulation to contain a target human milk cream content. In some embodiments, the human milk cream is added in an amount sufficient for the topical formulation to contain or include at least 5%, 10%, 20%, 25%, 30%, 40%, or 50%, or between 1% and 75%, 5% and 50%, 10% and 50%, 10% and 30%, 10% and 20%, 30% and 50%, 40% and 60%, or 50% and 75% human milk cream, e.g., w/w or w/v, each inclusive. In certain embodiments, the human milk cream is added or incorporated into the topical formulation to contain at least or about between 10% and 30% human milk cream, e.g., by w/v or w/w, inclusive. In particular embodiments, the human milk cream is added or incorporated into the topical formulation to contain about 20% human milk cream, e.g., by w/v or w/w.

In some embodiments, human milk cream is added or incorporated into the topical formulation to contain a target fatty acid, fat, or lipid content. In some embodiments, the human milk cream is added in an amount sufficient for the topical formulation to contain or include at least 5%, 10%, 20%, 25%, or 30%, or between 5% and 25%, 10% and 20%, 1% and 20%, or 5% and 15% human milk lipid, fat, or fatty acid content, e.g., by w/w or w/v. In particular embodiments, the human milk cream is added or incorporated into the topical formulation to contain or include about 20% human milk lipid, fat, or fatty acids. In certain embodiments, the human milk cream is added or incorporated into the topical formulation to contain or include about 10% human milk lipid, fat, or fatty acids.

In certain embodiments, human milk permeate is added or incorporated into the topical formulation to contain a target human milk permeate content. In some embodiments, the human milk permeate is added in an amount sufficient for the topical formulation to contain or include at least 1%, 10%, 20%, 30%, 40%, 50%, 60%, or 70% human milk permeate, e.g., w/w or w/v. In particular embodiments, the human milk permeate is formulated into a topical formulation by substituting an amount of human milk permeate for the amount of water that is typically added to a standard topical formulation, e.g., a typical or known formulation for an oil-in water emulsion, a water-in-oil emulsion, a nanoemulsion, an emulsified gel, an oleaginous ointment, a hydrocolloid system, a cream preparation, or any other pharmaceutically acceptable formulation suitable for use as a topical formulation. Thus, if water is typically added to produce a topical formulation, e.g., an oil-in water emulsion, a water-in-oil emulsion, a nanoemulsion, an emulsified gel, an oleaginous ointment, a hydrocolloid system, or a cream preparation, the amount of human milk permeate is added in place of the water to produce the topical formulation. In particular embodiments, the permeate contains at least 0.5% HMO w/v or w/w.

In certain embodiments, an HMO composition, such as an HMO composition derived from human milk permeate as described herein, is added or incorporated into the topical formulation to contain a target HMO composition content. In some embodiments, the HMO composition is added in an amount sufficient for the topical formulation to contain or include at least 1%, 10%, 20%, 30%, 40%, 50%, 60%, or 70% of the HMO composition, e.g., w/w or w/v. In particular embodiments, the HMO composition is formulated into a topical formulation by substituting an amount of human milk permeate for the amount of water that is typically added to a standard topical formulation, e.g., a typical or known formulation for an oil-in water emulsion, a water-in-oil emulsion, a nanoemulsion, an emulsified gel, an oleaginous ointment, a hydrocolloid system, a cream preparation, or any other pharmaceutically acceptable formulation suitable for use as a topical formulation. In particular embodiments, the HMO composition contains at least 5% HMO w/v or w/w.

In some embodiments, human milk retentate is added or incorporated into the topical formulation to contain a target human milk protein content. In some embodiments, the human milk retentate is added in an amount sufficient for the topical formulation to contain or include at least with at least 1%, 5%, 10%, 20%, 25%, 30%, 40%, 50%, 60%, or 70% or more human milk retentate, e.g., w/w or w/v. In certain embodiments, the human milk retentate is added or incorporated into the topical formulation to contain at least or about between 10% and 30% human milk retentate, e.g., by w/v or w/w. In particular embodiments, the human milk retentate is added or incorporated into the topical formulation to contain between 0.1% and 20%, 1% and 5%, or 0.1% and 2% human milk retentate e.g., by w/v or w/w.

In some embodiments, human milk retentate is added or incorporated into the topical formulation to contain a target protein content. In some embodiments, the human milk retentate is added in an amount sufficient for the topical formulation to contain or include at least 0.1%, 0.5%, 1%, 2%, 3%, 4%, or 5% human milk protein content, e.g., by w/w or w/v.

In some embodiments, the topical formulation is formulated with human milk cream or a sub-fraction thereof, e.g., a butteroil fraction or a milk fat globule membrane solids fraction. In certain embodiments, the topical formulation is formulated with human milk cream or a sub-fraction thereof, and human milk permeate or a portion or fraction thereof. In some embodiments, the topical formulation is formulated with human milk cream, or a sub-fraction thereof, to contain from 10% to 30% w/w fat, fatty acid, or lipid content. In certain embodiments, the topical formulation is formulated with one or more excipients, such as one or more emulsifiers, one or more preservatives, one or more thickening agents, one or more surfactants, one or more antioxidants, and/or one or more hydrocolloids. In particular embodiments, the topical formulation is formulated by adding the human milk cream or a sub-fraction thereof in an amount sufficient for a final concentration of fat, fatty acid, or lipids of 10% to 30% w/w with human milk permeate and the one or more excipients; mixing the contents; and then adding a hydrocolloid or gum while mixing. In particular embodiments, the contents are mixed with a mechanical disruptor at a speed of at least 500 rpm, 1,000 rpm, 5,000 rpm, 10,000 rpm, 25000 rpm, 50,000 rpm or more for at least 5 minutes, 15 minutes, 30 minutes, 60 minutes, 90 minutes, 120 minutes, 150 minutes, 180 minutes, 2 hours, 3 hours, 4 hours, 6 hours, 8 hours, or 12 hours. In particular embodiments, the xanthan gum is added while mixing at a low speed, e.g., at or less than 5,000 rpm, 2,000 rpm, 1,000 rpm, or 500 rpm.

In particular embodiments, the topical formulation is formulated with human milk cream or a sub-fraction thereof, e.g., a butteroil fraction or a milk fat globule membrane solids sub-fraction, human milk permeate or a fraction or portion thereof, and one or more excipients that are or include an emulsifier, a preservative, a thickening agent, a nonionic surfactant, an antioxidant, and/or a hydrocolloid. In certain embodiments, the topical formulation is formulated by adding the human milk cream, or a sub-fraction thereof, in an amount sufficient for a final concentration of fat, fatty acid, or lipids of 10% to 30% w/w with human milk permeate, and an emulsifier, a preservative, a thickening agent, a nonionic surfactant, an antioxidant, mixing the contents, and then adding a hydrocolloid while mixing. In certain embodiments, the emulsifier is or includes TWEEN 80 or polysorbate 80. In some embodiments, the preservative is or includes phenoxyethanol. In certain embodiments, the thickening agent is or includes liquid paraffin. In some embodiments, the antioxidant is or includes alpha tocopherol. In particular embodiments, the hydrocolloid is or includes xanthan gum. In particular embodiments, the topical formulation is formulated by adding the human milk cream, or a sub-fraction thereof, in an amount sufficient for a final concentration of fat, fatty acid, or lipids of 10% to 30% w/w with human milk permeate and Tween 80, phenoxyethanol, liquid paraffin, Span 83, and alpha tocopherol; mixing the contents; and then adding xanthan gum while mixing. In particular embodiments, the contents are mixed with a mechanical disruptor at a speed of at least 500 rpm, 1,000 rpm, 5,000 rpm, 10,000 rpm, 25000 rpm, 50,000 rpm or more for at least 5 minutes, 15 minutes, 30 minutes, 60 minutes, 90 minutes, 120 minutes, 150 minutes, 180 minutes, 2 hours, 3 hours, 4 hours, 6 hours, 8 hours, or 12 hours. In particular embodiments, the xanthan gum is added while mixing at a low speed, e.g., at or less than 5,000 rpm, 2,000 rpm, 1,000 rpm, or 500 rpm.

In particular embodiments, the topical formulation is formulated with human milk cream and human milk permeate. In some embodiments, human milk cream is added or incorporated into the topical formulation to contain a target fat, fatty acid, or lipid content in an amount sufficient for the topical formulation to contain or include at least 5%, 10%, 20%, 25%, or 30%, or between 5% and 25%, 10% and 20%, 1% and 20%, or 5% and 15% human milk lipid, fat, or fatty acid content, e.g., by w/w or w/v. In certain embodiments, human milk cream is added or incorporated into the topical formulation to contain a target human milk fat content of at least 5%, 10%, 20%, 25%, or 30%, or between 5% and 25%, 10% and 20%, 1% and 20%, or 5% and 15% human milk fat, e.g., by w/w. In particular embodiments, the human milk permeate is formulated into the topical formulation by substituting an amount of human milk permeate for the amount of water that is typically added to a standard topical formulation, e.g., a typical or known formulation for an oil-in water emulsion, a water-in-oil emulsion, a nanoemulsion, an emulsified gel, an oleaginous ointment, a hydrocolloid system, a cream preparation, or any other pharmaceutically acceptable formulation suitable for use as a topical formulation. In particular embodiments, the topical formulation is formulated with an amount of human milk cream sufficient for the topical formulation to contain about 10% human milk lipid, fat, or fatty acids, and with human milk permeate in place of water, e.g., in an amount sufficient for the topical formulation contain between 0.1% and 0.5% HMO by w/w or w/v. In some embodiments, the topical formulation is formulated with an amount of human milk cream sufficient for the topical formulation to contain about 20% human milk fat and with human milk permeate in place of water, e.g., in an amount sufficient for the topical formulation contain between 0.1% and 0.5% HMO (e.g., by w/w).

### IV. Formulations Containing Human Milk Fractions, Portions, or Components

In some embodiments, the provided topical formulation may be formulated as a cosmetic or pharmaceutical composition suitable for topical administration to a subject, e.g., such as locally and/or directly to the skin. In certain embodiments, the topical formulation may be formulated into one or more of a lotion, a gel, emulsion, an oil-in water emulsion, a water-in-oil emulsion, a nanoemulsion, an emulsified gel, an oleaginous ointment, a hydrocolloid system, a cream preparation, a serum, a nanoemulsion, or a powder. In certain embodiments, the topical formulation may be formulated as an oil-in water emulsion, a nanoemulsion, an emulsified gel, an oleaginous ointment, a hydrocolloid system, or a cream preparation.

In some embodiments, the topical formulation is or remains stable, e.g., does not undergo phase separation, changes in chemical composition, oxidation of fatty acids, changes in color, smell, or appearance, or changes in viscosity, over a period of time. In some embodiments, the topical formulation is or remains stable when refrigerated, e.g., stored under 10° C. or between or between about 2° C. and 8° C., such as at about 4° C. In particular embodiments, the topical formulation is stable for at least 1 week, 2 weeks, 3 weeks, 4 weeks, 5 weeks, 6 weeks, 7 weeks, 8 weeks, 9 weeks, 10 weeks, 12 weeks, 14 weeks, 2 months, 3 months, 4 months, 6 months, 12 months, 18 months, 1 year, 2 years, or 5 years when refrigerated, e.g., stored under 10° C. or between or between about 2° C. and 8° C., such as at about 4° C. In some embodiments, the topical formulation is stable for at least 3 months when refrigerated. In particular embodiments, the topical formulation is stable for at least 6 months when refrigerated. In some embodiments, the topical formulation is stable for at least one year when refrigerated.

In particular embodiments, the topical formulation is or remains stable when stored at room temperature, e.g., stored between or between about 20° C. and 30° C., such as at between or between about 23° C. and 27° C. or at or at about 25° C. In some embodiments, the topical formulation is stable for at least 1 week, 2 weeks, 3 weeks, 4 weeks, 5 weeks, 6 weeks, 7 weeks, 8 weeks, 9 weeks, 10 weeks, 12 weeks, 14 weeks, 2 months, 3 months, 4 months, 6 months, 12 months, 18 months, 1 year, 2 years, or 5 years at room temperature e.g., stored between or between about 20° C. and 30° C., such as at between or between about 23° C. and 27° C. or at about 25° C. In some embodiments, the topical formulation is stable for at least 3 months at room temperature. In some embodiments, the topical formulation is stable for at least 6 months at room temperature. In certain embodiments, the topical formulation is stable for at least one year at room temperature.

In some embodiments, the topical formulation contains one or more portions, fractions, or components of human milk and is formulated or composed as a cream preparation, an oil-in-water emulsion, a water-in-oil emulsion, a nanoemulsion, a hydrocolloid system, an emulsified gel, or an oleaginous ointment. In some embodiments, the topical formulation includes human milk cream or a portion or component thereof. In certain embodiments, the topical formulation includes human milk permeate, e.g., from the ultrafiltration of human milk skim, or a portion or component thereof. In certain embodiments, the topical formulation includes human milk cream and human milk permeate and is formulated or composed as a cream preparation, an oil-in-water emulsion, a water-in-oil emulsion, a nanoemulsion, a hydrocolloid system, an emulsified gel, or an oleaginous ointment.

In some embodiments, the topical formulation may be delivered by a variety of applicators appropriate for localized and general application. Such applicators can include droppers, applicator wands, cotton swabs, or any other suitable device. It is to be appreciated that the compositions herein may also be applied locally and/or directly by using one's finger or in other conventional or routine manners. In some embodiments, the topical formulation is administered locally, e.g., to all or a region of the skin or mucous membrane. In certain embodiments, the topical formulation is administrated directly, e.g., to all or a region of the skin or mucous membrane.

In certain embodiments, the topical formulation includes one or more excipients, such as including, but not limited to a hydrocolloid, an emulsifier, a structuring agent, an antioxidant, a silicone containing compound, a thickening agent, a preservative, an additional skin conditioning agent, an emollient, a humectant, or an essential oil.

In some embodiments, the topical formulation includes one or more excipients that are or include one or more hydrocolloids. Hydrocolloids, also referred to herein and in the art as gums, are in some aspects hydrophilic polymers of vegetable, animal, microbial or synthetic origin, that generally contain many hydroxyl groups. In some embodiments, the hydrocolloid is or includes a polyelectrolyte. Hydrocolloids are in certain aspects well-known in the art and include, but are not limited to, xanthan gum, cellulose, carboxymethyl cellulose, hydroxyethyl cellulose, hydroxypropyl cellulose, methyl and ethyl cellulose, natural gums, natural starches, and deionized starches (e.g., starch octenyl succinate). In certain embodiments, non-limiting examples of gums that can be used with the provided compositions and formulations include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, nano gum, potassium alginate, potassium carrageenan, propylene glycol alginate, sclerotium gum, sodium carboxymethyl dextran, sodium carrageenan, tragacanth gum, xanthan gum, and mixtures thereof.

In particular embodiments, the provided topical formulation includes one or more excipients that is or includes an emulsifier. In certain embodiments, addition of the emulsifier reduces the interfacial tension between phases and improves the formulation and stability of the emulsion. In certain embodiments, the emulsifiers include nonionic, cationic, anionic, and zwitterionic emulsifiers (See McCutcheon in “Detergents and Emulsifiers” North American Ed. 1986; U.S. Pat. Nos. 5,011,681; 4,421,769; 3,755,560). Suitable emulsifiers include, but are not limited to, esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20, also known as Tween 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof. In some embodiments, the emulsifier is or includes paraffin, e.g., soft white paraffin, liquid paraffin, cetomacrogol 1000, Tween 60, span 60, Brij S2 (Brij 72), Brij 5721 (Brij 721), and/or ascorbyl palmitate.

In some embodiments, the topical formulation includes and/or is formulated with a surfactant. In some embodiments, the surfactant is or includes a non-ionic surfactant. Nonionic surfactants may include, but are not necessarily limited to, alkyl polyglycosides, sorbitan esters, methyl glucoside esters, alcohol ethoxylates, or polyglycol esters. In some embodiments, the nonionic surfactant is Span 83 or Span 60. In some embodiments, the surfactant is or includes a cationic surfactant. Cationic surfactants may include, but are not limited to, arginine methyl esters, alkanolamines and alkylenediamides. In particular embodiments, the surfactant is an anionic surfactant. Anionic surfactants may include, but are not limited to, alkali metal alkyl sulfates, alkyl or alkylaryl sulfonates, linear or branched alkyl ether sulfates and sulfonates, alcohol polypropoxylated and/or polyethoxylated sulfates, alkyl or alkylaryl disulfonates, alkyl disulfates, alkyl sulphosuccinates, alkyl ether sulfates, linear and branched ether sulfates, and mixtures thereof.

In certain embodiments, the topical formulation contains one or more excipients that are or include a structuring agent. In certain embodiments, the structuring agent assists in providing rheological characteristics to the composition and/or contribute to the composition's stability. In some embodiments, the structuring agent functions as an emulsifier or surfactant. Suitable structuring agents for use as excipients include, but are not limited to, stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

In some embodiments, the topical formulation contains one or more excipients that are or include an antioxidant. In some aspects, the antioxidants may include, but are not limited to, one or more of acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

In some embodiments, the topical formulation contains one or more excipients that are or include a silicone containing compound. In some aspects, silicone containing compounds include any member of a family of polymeric products whose molecular backbone is made up of alternating silicon and oxygen atoms with side groups attached to the silicon atoms. In some aspects, the —Si—O— chain lengths, side groups, and crosslinking, silicones of the silicone containing compounds can be synthesized into a wide variety of materials. They can vary in consistency from liquid to gel to solids.

In certain embodiments, silicone containing compounds include, but are not limited to silicone oils (e.g., volatile and non-volatile oils), gels, and solids. In certain embodiments, the silicon containing compound is or includes a silicone oil such as a polyorganosiloxane. In particular embodiments, the polyorganosiloxane is or includes dimethicone, cyclomethicone, polysilicone-11, phenyl trimethicone, trimethylsilylamodimethicone, stearoxytrimethylsilane, or mixtures of these and other organosiloxane materials. In certain embodiments, polyoranosiloxanes and organosiloxame materials may be mixed in any given ratio in order to achieve the desired consistency and application characteristics depending upon the intended application (e.g., to a particular area such as the skin, hair, or eyes). In some embodiments, a “volatile silicone oil” is or includes a silicone oil have a low heat of vaporization, i.e. normally less than about 50 cal per gram of silicone oil. In some aspects, suitable silicone oils may include, but are limited to: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Mich. Cyclomethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. In certain embodiments, suitable volatile silicone oils may also include those available from General Electric Co., Silicone Products Div., Waterford, N.Y. and SWS Silicones Div. of Stauffer Chemical Co., Adrian, Mich.

In some embodiments, the topical formulation is or contains one or more thickening agents. In certain embodiments, the thickening agents may include but are not limited to carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerytritol (e.g., Carbopol™ 900 series from B.F. Goodrich). In certain embodiments, the thickening agents are or include carabopol, carrageenan, HPC, paraffin wax, PEG 4000, sodium alginate, and Avacel CL-611.

Non-limiting examples of crosslinked polyacrylate polymers include cationic and nonionic polymers. Examples are described in U.S. Pat. Nos. 5,100,660; 4,849,484; 4,835,206; 4,628,078; 4,599,379.

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C10-C30 straight chain or branched chain alkyl group through an ether linkage. Typically, these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three unit.

In particular embodiments, the topical formulation contains one or more preservatives. Non-limiting examples of preservatives that can be used in the formulation of the provided topical formulations include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal or combinations thereof.

In certain embodiments, the topical formulation is formulated with an additional skin conditioning agent. In some aspects, such an agent may comprise a substance that enhances the appearance of dry or damaged skin, as well as a material that may adhere to the skin to reduce flaking, restore suppleness, and generally improve the appearance of skin. Specific examples of skin conditioning agents include, without limitation, acetyl cysteine, N-acetyl dihydrosphingosine, acrylates/behenyl acrylate/dimethicone acrylate copolymer, adenosine, adenosine cyclic phosphate, adenosine phosphate, adenosine triphosphate, alanine, albumen, algae extract, allantoin and derivatives, aloe barbadensis extracts, amyloglucosidase, arbutin, arginine, bromelain, buttermilk powder, butylene glycol, calcium gluconate, carbocysteine, carnosine, beta-carotene, casein, catalase, cephalins, ceramides, Chamomilla recutita (matricaria) flower extract, cholecalciferol, cholesteryl esters, coco-betaine, corn starch modified, crystallins, cycloethoxymethicone, cysteine DNA, cytochrome C, darutoside, dextran sulfate, dimethicone copolyols, dimethylsilanol hyaluronate, elastin, elastin amino acids, ergocalciferol, ergosterol, fibronectin, folic acid, gelatin, gliadin, beta-glucan, glucose, glycine, glycogen, glycolipids, glycoproteins, glycosaminoglycans, glycosphingolipids, horseradish peroxidase, hydrogenated proteins, hydrolyzed proteins, jojoba oil, keratin, keratin amino acids, kinetin, lactoferrin, lanosterol, lecithin, linoleic acid, linolenic acid, lipase, lysine, lysozyme, malt extract, maltodextrin, melanin, methionine, niacin, niacinamide, oat amino acids, oryzanol, palmitoyl hydrolyzed proteins, pancreatin, papain, polyethylene glycol, pepsin, phospholipids, phytosterols, placental enzymes, placental lipids, pyridoxal 5-phosphate, quercetin, resorcinol acetate, riboflavin, saccharomyces lysate extract, silk amino acids, sphingolipids, stearamidopropyl betaine, stearyl palmitate, tocopherol, tocopheryl acetate, tocopheryl linoleate, ubiquinone, Vitis vinifera (grape) seed oil, wheat amino acids, xanthan gum, zinc gluconate, or combinations thereof. In some embodiments, a skin conditioning agent, other than those listed above, may also be incorporated or formulated into the provided topical formulations, as is readily appreciated by those skilled in the art.

In certain embodiments, the topical formulation contains one or more emollients. In some aspects, an emollient is an ingredient that may help skin maintain a soft, smooth, and pliable appearance. In some embodiments, the emollient may include, but is not limited to, acetyl arginine, acetylated lanolin, algae extract, apricot kernel oil polyethylene glycol-6 esters, avocado oil polyethylene glycol-11 esters, bis-polyethylene glycol-4 dimethicone, butoxyethyl stearate, C₁₈ C₃₆ acid glycol ester, C₁₂ C₁₃ alkyl lactate, caprylyl glycol, cetyl esters, cetyl laurate, coconut oil polyethylene glycol-10 esters, di-C₁₂ C₁₃ alkyl tartrate, diethyl sebacate, dihydrocholesteryl butyrate, dimethiconol, dimyristyl tartrate, disteareth-5 lauroyl glutamate, ethyl avocadate, ethylhexyl myristate, glyceryl isostearates, glyceryl oleate, hexyldecyl stearate, hexyl isostearate, hydrogenated palm glycerides, hydrogenated soy glycerides, hydrogenated tallow glycerides, isostearyl neopentanoate, isostearyl palmitate, isotridecyl isononanoate, laureth-2 acetate, lauryl polyglyceryl-6 cetearyl glycol ether, methyl gluceth-20 benzoate, mineral oil, myreth-3 palmitate, octyldecanol, octyldodecanol, Odontella aurita oil, 2-oleamido-1,3 octadecanediol, palm glycerides, polyethylene glycol avocado glycerides, polyethylene glycol castor oil, polyethylene glycol-22/dodecyl glycol copolymer, polyethylene glycol shea butter glycerides, phytol, raffinose, stearyl citrate, sunflower seed oil glycerides, tocopheryl glucoside, paraffin (e.g., soft white paraffin or liquid paraffin), or combinations thereof. Those skilled in the art will readily appreciate that emollients, other than those listed above, may also be formulated or incorporated into the provided topical formulations.

In some embodiments, the topical formulation is formulated with one or more humectants. In certain aspects, humectants are ingredients that may help maintain moisture levels in skin. Specific examples of humectants include, without limitation, acetyl arginine, algae extract, aloe barbadensis leaf extract, 2,3-butanediol, chitosan lauroyl glycinate, diglycereth-7 malate, diglycerin, diglycol guanidine succinate, erythritol, fructose, glucose, glycerin, honey, hydrolyzed wheat protein/polyethylene glycol-20 acetate copolymer, hydroxypropyltrimonium hyaluronate, inositol, lactitol, maltitol, maltose, mannitol, mannose, methoxy polyethylene glycol, myristamidobutyl guanidine acetate, polyglyceryl sorbitol, potassium PCA, propylene glycol, sodium PCA, sorbitol, sucrose, urea, or combinations thereof.

In certain embodiments, the topical formulation may include one or more essential oils. In certain embodiments, the essential oils include oils derived from herbs, flowers, trees, and other plants. Such oils are typically present as tiny droplets between the plant's cells, and can be extracted by several methods known to those of skill in the art (e.g., steam distilled, enfleurage (i.e., extraction by using fat), maceration, solvent extraction, or mechanical pressing). In some aspects, when these types of oils are exposed to air they tend to evaporate (i.e., a volatile oil). In certain embodiments, essential oils may be insoluble in water but soluble in alcohol, ether, fixed oils (vegetal), or other organic solvents. In certain embodiments, typical physical characteristics found in essential oils include boiling points that vary from about 160° to 240° C. and densities ranging from about 0.759 to about 1.096.

The topical formulation herein may include one or more sunscreen actives (or sunscreen agents) and/or ultraviolet light absorbers. Herein, “sunscreen active” collectively includes sunscreen actives, sunscreen agents, and/or ultraviolet light absorbers. Sunscreen actives include both sunscreen agents and physical sunblocks. Sunscreen actives may be organic or inorganic. Examples of suitable sunscreen actives are disclosed in Personal Care Product Council's International Cosmetic Ingredient Dictionary and Handbook, Thirteenth Edition, as “sunscreen agents.” Particularly suitable sunscreen actives are 2-ethylhexyl-p-methoxycinnamate (commercially available as PARSOL™ MCX), 4,4′-t-butyl methoxydibenzoyl-methane (commercially available as PARSOL™ 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxypropyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, menthyl anthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, zinc oxide, benzylidene camphor and derivatives thereof, titanium dioxide, and mixtures thereof.

In some embodiments, the topical formulation is adjusted to a target pH or apparent pH. In particular embodiments, the topical formulation contains a neutral pH or apparent pH. In some embodiments, the topical formulation contains a pH or apparent pH of between or between about 5.0 and 8.0, inclusive. In certain embodiments, the topical formulation contains a pH or nn apparent pH of between or between about 6.6 and 6.9, inclusive. In some embodiments, the composition contains an acidic pH or apparent pH, such as when the composition is formulated as a topical cream or cream preparation. In particular embodiments, the pH or apparent pH is between or between about 4.0 and 5.0 or 4.4 and 4.7, each inclusive. In some embodiments, the pH or apparent pH of the topical formulation is from 4.0 to 5.5. In certain embodiments, the pH or apparent pH of the topical formulation is or is about 5.5. In particular embodiments, the pH or apparent pH of the topical formulation is or is about 5.2.

In some embodiments, the topical formulation is filterable, e.g., to remove or reduce bioburden. In some embodiments, the topical formulation is filterable through a filter that is between or between about 0.1 μm and 1.0 μm in size, e.g., a filter that is or is about 0.45 μm or 0.2 μm in size. In some embodiments, the composition is filterable through a filter that is or is about 0.2 μm in size.

In certain embodiments, the topical formulation is or includes an emulsion, e.g., an oil-in-water emulsion. In some aspects, the oil-in-water emulsion contains an oil phase that is dispersed into an aqueous phase. In some embodiments, the aqueous phase is or contains human milk permeate, or a portion, fraction, or component thereof. In certain embodiments, the aqueous phase is or contains human milk oligosaccharides. In certain embodiments, the oil phase contains human milk cream. In certain embodiments, the oil phase contains human milk globules. In particular embodiments, the oil phase contains human milk lipids. In certain embodiments, oil-in-water emulsion contains oil droplets (e.g., 1-1,000 nm in diameter). In some embodiments, the composition contains oil droplets, e.g., droplets containing or composed of human milk cream, lipids, and/or globules, or fractions or portions thereof. In certain embodiments, the droplets are relatively uniform in size, e.g., at least 50%, 60%, 70%, 75%, 80%, 85%, 90%, or 95% of the droplets fall within 2, 1.5, 1, or 0.5 standard deviations from the average or median droplet size. In some embodiments, the average or median droplet size is between 100 nm and 2 μm. In particular embodiments, the average or median droplet size is between 250 nm and 1,000 nm, 500 nm and 2,000 nm, or 500 nm and 1,000 nm.

In certain embodiments, the topical formulation is or includes an emulsion, e.g., a nanoemulsion, such as an emulsion having nanometer sized droplets (e.g., 1-1,000 nm in diameter). In some embodiments, the composition contains oil droplets, e.g., droplets containing or composed of human milk lipids or human milk globules or portions or fractions thereof. In certain embodiments, the droplets are relatively uniform in size, e.g., at least 50%, 60%, 70%, 75%, 80%, 85%, 90%, or 95% of the droplets fall within 2, 1.5, 1, or 0.5 standard deviations from the average or median droplet size. In some embodiments, the average or median droplet size is less than or less than about 1,000 nm, 750 nm, 500 nm, 400 nm, 300 nm, 200 nm, or 100 nm in diameter. In particular embodiments, the average or mediate droplet size is between or between about 100 nm and 250 nm in diameter. In certain embodiments, the average or mean droplet size is or is about 200 nm in diameter.

In certain embodiments, the topical formulation is or includes a hydrocolloid system, e.g., containing human milk cream or lipids. In some embodiments, the hydrocolloid system is formulated with or contains between or between about 50% and 75% (inclusive) human milk cream or a portion or fraction derived from or originating from human milk cream. In some embodiments, the hydrocolloid system includes one or more hydrocolloids. In certain embodiments, the hydrocolloid system includes one or more thickening agents. In particular embodiments, the hydrocolloid system includes one or more antioxidants. In certain embodiments, the hydrocolloid system includes one or both of xanthan gum and HPMC hydrocolloids, and one or more thickening agents selected from carabopol, carrageenan, HPC, paraffin wax, PEG 4000, sodium alginate, and Avacel CL-611. In some embodiments, the hydrocolloid systems are or include formulations containing 50% human milk cream and one or more hydrocolloids selected from xanthan gum, sodium alginate, Protanal HF120PBS, and Avicel CL-611. In some embodiments, benzyl alcohol is added is a preservative, and ascorbyl palmitate and alpha-tocopherol are added as antioxidants to the hydrocolloid system.

In some embodiments, the provided topical formulation is or includes a cream preparation. In some embodiments, the cream preparation contains between or between about 5% and 20%, inclusive, human milk cream is formulated from aqueous and oil phases. In some embodiments, the aqueous phases are composed of or includes one or more of xanthan gum, cetostearyl alcohol, phenoxyethanol, benzyl alcohol, or steric acid. In certain embodiments, the lipid phase contains or includes one or more of paraffin, e.g., soft white paraffin, liquid paraffin, cetomacrogol 1000, Tween 60, span 60, Brij S2 (Brij 72), Brij 5721 (Brij 721), ascorbyl palmitate, or ascorbic acid. In some embodiments, the cream preparation includes one or more antioxidants.

In certain embodiments, the provided composition is or includes an emulsified gel composition. In certain embodiments, the emulsified gel composition contains between or between about 5% and 20% human milk cream. In certain embodiments, the emulsified gel contains an aqueous phase and an oil phase. In particular embodiments, the aqueous phase includes one or both of Tween 80 and benzyl alcohol. In certain embodiments, the oil phase includes the human milk cream and one or both of liquid paraffin and SPAN 83. In some embodiments, the emulsified gel also includes one or more antioxidants. In particular embodiments, the emulsified gel includes xanthan gum as a gelling agent.

In some embodiments, the composition is formulated as an oleaginous ointment composition. In some embodiments, the oleaginous ointment contains between or between about 5% and 20% human milk cream, inclusive. In some embodiments, the oleaginous ointment contains one or more liquid paraffin, IPM, crodamol GRCC, castor oil, white soft paraffin, cetomacrogol 1000, and cetostearyl alcohol.

In certain embodiments, the topical formulation is or includes an oil-in-water emulsion. In some embodiments, the oil-in-water emulsion contains an aqueous phase and an oil phase. In some embodiments, the oil-in-water emulsion contains human milk cream. In certain embodiments, the oil-in-water emulsion contains homogenized human milk cream. In certain embodiments the oil-in-water emulsion contains human milk lipids.

In particular embodiments, the aqueous phase contains human milk permeate, e.g., permeate resulting from the ultrafiltration of human skim milk. In certain embodiments, the aqueous phase contains human milk oligosaccharides. In some embodiments, the aqueous phase contains human milk cream. In particular embodiments, the aqueous phase contains human milk fat, fatty acids, and/or lipids. In particular embodiments, the aqueous phase includes one or more excipients that include an emulsifier. In some embodiments, the emulsifier is a polysorbate, e.g., polysorbate 80 (Tween 80). In certain embodiments, aqueous phase contains one or more excipients that are or include a preservative. In certain embodiments, the preservative is phenoxyethanol. In certain embodiments, the oil-in-water emulsion contains an aqueous phase that is or includes human milk permeate, e.g., resulting from the ultrafiltration of human skim milk, an emulsifier, and a preservative. In some embodiments, the oil-in-water emulsion contains an aqueous phase that is or includes human milk oligosaccharides, an emulsifier, and a preservative.

In certain embodiments, the oil phase includes one or more excipients that are or include an emulsifier. In particular embodiments, the emulsifier is or includes Span 83. In some embodiments, the oil phase includes one or more excipients that are or include an emollient. In some embodiments, the emollient is or includes paraffin, e.g., liquid paraffin. In various embodiments, the oil phase is or includes an antioxidant. In particular embodiments, the oil phase includes alpha tocopherol. In some embodiments, the oil phase contains liquid paraffin, Span 83, and alpha tocopherol.

In some embodiments, the oil-in-water emulsion is formulated by combining the aqueous phase and oil phase mixtures, adding human milk cream, and adding a thickening agent. In some embodiments, the human milk cream is homogenized prior to adding. In certain embodiments, the human milk cream is added to achieve a target concentration of human milk fatty acids, fat, or lipids. In some embodiments, the target concentration is between 1% and 20%, 5% and 15%, 7.5% and 12.5%, or 8% and 12% total fat, fatty acid, or lipids by weight over volume (w/v) of the oil-in-water emulsion. In some embodiments, the thickening agent is a gum. In certain embodiments, the gum is a xanthene gum.

In some embodiments, the topical formulation is formulated with human milk cream or a sub-fraction thereof, e.g., a butteroil fraction or a milk fat globule membrane solids sub-fraction, human milk permeate or a fraction or portion thereof, and one or more excipients. In particular embodiments, the topical formulation is formulated with human milk cream, human milk permeate, and one or more excipients. In certain embodiments, the topical formulation is formulated with human milk butteroil, human milk permeate, and one or more excipients. In certain embodiments, the topical formulation contains a human milk fat content of 10% to 30% w/w. In particular embodiments, the topical formulation contains at least 0.1%, 0.3%, 0.5%, 1%, or 3% human milk oligosaccharides by W/W.

In certain embodiments, the one or more excipients are or include one or more of an emulsifier, a preservative, a thickening agent, a nonionic surfactant, an antioxidant, and/or a hydrocolloid. In particular embodiments, the topical formulation is formulated with human milk cream or a sub-fraction thereof, human milk permeate or a fraction or portion thereof, and excipients that include an emulsifier, a preservative, a thickening agent, a nonionic surfactant, an antioxidant, and a hydrocolloid. In certain embodiments, the emulsifier is or includes Tween 80 or polysorbate 80. In some embodiments, the preservative is or includes phenoxyethanol. In certain embodiments, the thickening agent is or includes liquid paraffin and/or glycerin. In some embodiments, the antioxidant is or includes alpha tocopherol. In particular embodiments, the hydrocolloid is or includes xanthan gum.

### V. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the described or claimed subject matter. This applies regardless of the breadth of the range.

Throughout this disclosure, ranges that are presented or expressed as “between” two endpoints, e.g., “between A and B” are understood to include the endpoints, e.g. “A” and “B”, unless otherwise indicated.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. In some aspects, “about” a value means within a range of ±25%, ±10%, ±5%, ±1%, ±0.1%, or ±0.01% of the value.

Unless otherwise indicated, the term “topical formulation” is used herein to generally include a composition or formulation suitable for topical delivery or administration, e.g., to the skin or mucosa.

As used herein the term “pharmaceutical composition” means, for example, a mixture or formulation containing a specified amount, e.g. a therapeutically effective amount, of an active ingredient such as a human milk fraction, in a pharmaceutically acceptable carrier to be administered to a mammal, e.g., a human.

As used herein the term “pharmaceutically acceptable” refers to those compounds, materials, compositions and/or dosage forms, which are, within the scope of sound medical judgment, suitable for contact with the tissues of mammals, especially humans, without excessive toxicity, irritation, allergic response, and other problem complications commensurate with a reasonable benefit/risk ratio. Such reasonable benefit/risk ratios may be determined by of skill as a matter of routine.

By “human milk oligosaccharide(s)” (also referred to herein as “HMO(s)”) is meant a family of structurally diverse unconjugated glycans that are found in human breast milk. Human milk oligosaccharides are carbohydrates that contain lactose at the reducing end and, typically, fucose, sialic acid or N-acetylglucosamine at the non-reducing end (Morrow et al., J. Nutri. 2005 135:1304-1307).

The terms “mammalian milk lipid,” “mammalian milk protein,” or “mammalian milk oligosaccharide” as used herein refer to a lipid, protein, or oligosaccharide, respectively, that is naturally found in or originates from mammalian milk. Likewise, the terms “human milk lipid” or “human milk protein” refer herein to a lipid or protein, respectively, that is naturally found in or originates from human milk.

By “permeate” is meant a portion of milk (e.g. pooled human milk) obtained as the filter flow-through product during ultrafiltration. Specifically, the liquid that flows through after the retention of proteins and lipids during ultrafiltration (e.g. through a filter of about 1-10,000 kDa). The liquid that passes through this ultrafiltration process is referred to as permeate. The retentate of this process concentrates human milk protein which may then be used to create other life-saving formulations, for example, to make human milk fortifier compositions, such as those described in, U.S. Pat. No. 8,377,455. Thus, in contrast to methods that rely on protein precipitation with solvents, which may contaminate the HMO product, the use of ultrafiltration to obtain a substantially protein-free starting material as used herein, preserves the remainder of the valuable macronutrients in human milk while avoiding the use of organic solvents.

By “milk” is meant the fluid that is produced by the mammary gland of a mammal and expressed by the breast. Milk includes all lactation products including, but not limited to colostrum, whole milk and skim milk taken at any point post parturition or during the cycle of lactation, in the production of hind milk and foremilk. Unless otherwise specified, as used herein “milk” refers typically to whole human milk.

By “whole milk” is meant milk (e.g. pooled human milk) from which no fat has been removed.

By “skim milk” is meant milk (e.g. pooled human milk) from which at least 75% of fat has been removed or alternatively, milk that has been subject to centrifugation to remove the fat.

The term “essentially free” of a substance, e.g., of a non-human milk lipid or non-human milk oligosaccharide, means that none of the substance, e.g., no non-human milk lipid or non-human milk oligosaccharide, has been intentionally added, or that no more than trace amounts of the substance, e.g., non-human milk lipid or non-human milk oligosaccharide, is present.

Unless otherwise indicated, “human milk cream” or “human cream” is meant the portion of whole human milk separated from the human skim milk. Typically, the cream comprises long chain, medium chain, and short chain fatty acids at a concentration higher than that of skim milk obtained from the same preparation.

By “substantially” as in “substantially reduced lactose- and/or mineral content” is meant that the reduction in the level of minerals and/or lactose represents a statistical difference when compared to concentrated permeate that has not been subject to the current methods. By way of example, in some embodiments, the HMO compositions with substantially reduced lactose comprise lactose levels of <5%.

By “consisting essentially” of, as used herein refers to compositions containing particular recited components while excluding other major bioactive factors. For example, a composition consisting essentially of HMOs, would exclude such things as intact protein, fat, or exogenously added material, but may contain other inert or trace materials, such as water, acceptable levels of certain salts, microRNAs, or exosomes, for example.

### EXAMPLES

The following examples are included for illustrative purposes only and are not intended to limit the scope of the invention.

### Example 1: Generation of Cream, Permeate, and Retentate Fractions of Human Milk

Human milk pooled from individual donors was separated into cream, permeate, and retentate fractions. The pooled whole human milk was filtered through a 200 μm filter, pasteurized at a temperature of at least 63° C. for 30 minutes and then cooled to between 2° C. and 8° C. The milk was then transferred to a centrifuge to separate the cream from the skim. The cream was collected and stored at −20° C. and the skim milk was ultra-filtered with a 10 kDa membrane. The material that did not pass through the filter was collected as the retentate fraction, and material that passed through the filter was the permeate fraction.

### Example 2: Effects of Human Milk Fractions on a Three-Dimensional In Vitro Healthy Human Skin Model

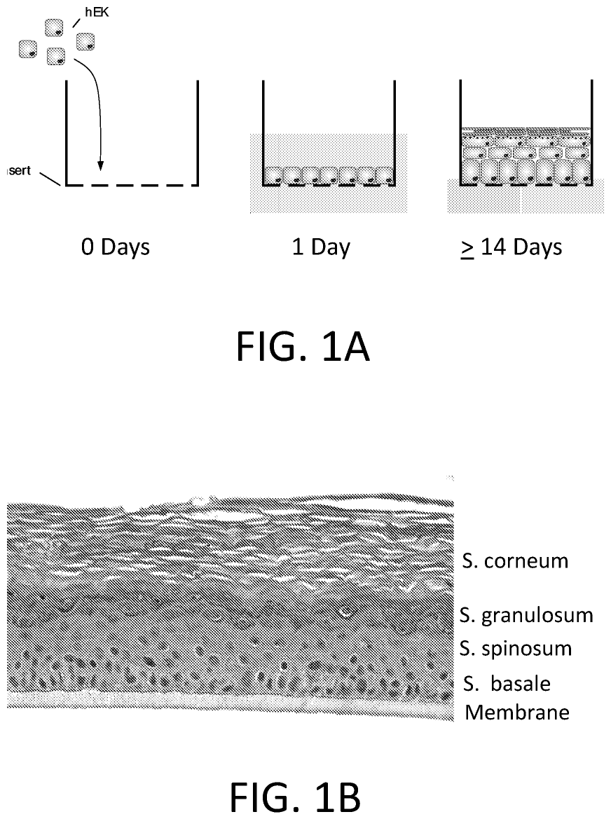

Effects on skin of human milk fractions generated as described in example 1 were evaluated with a three-dimensional in vitro human skin model. The in vitro skin model was similar to those described in PCT Application No. WO2001092477 (hereby incorporated by reference in its entirety). Preparations of the human skin model were generated by embedding human dermal fibroblasts into a biomatrix containing matrix proteins and submerged in culture media as illustrated in FIG. 1A. After approximately one day, the media volume was reduced to convert the preparation to an air-liquid interface culture. Keratinocytes were then seeded on top of the fibroblast layer which served as a scaffold. The cells were cultured for two to three weeks under conditions that permitted the keratinocytes to differentiate to a multilevel epidermis with stratum basale, stratum spinosum, stratum granulosum, and stratum corneum layers as well as a functional basal membrane composed of matrix proteins as shown in FIG. 1B.

Human milk cream, retentate, and permeate fractions generated as described in Example 1 were formulated to concentrations of 1%, 5%, 10%, and 30% in a PBS vehicle containing 1% DMSO. A vehicle only condition served as a negative control, and a plant extract that was previously shown to increase barrier function in the in vitro skin preparations was used as a control. The formulations were applied to the in vitro skin preparations once a day for three days. Once each day, the transepithelial/transendothelial electrical resistance (TEER) of the in vitro human skin preparations was measured at a reference frequency at 1,000 Hz by impedance spectroscopy to assess barrier function. Media was also collected daily and assessed for levels of IL-8 by ELISA, glucose consumption with a photometric hexokinase assay, and lactate dehydrogenase (LDH) with an enzymatic photometric assay. After 72 hours, the in vitro human skin preparations were sectioned and stained with hematoxylin and eosin (H&E) or evaluated with an MTT assay to assess cellular viability.

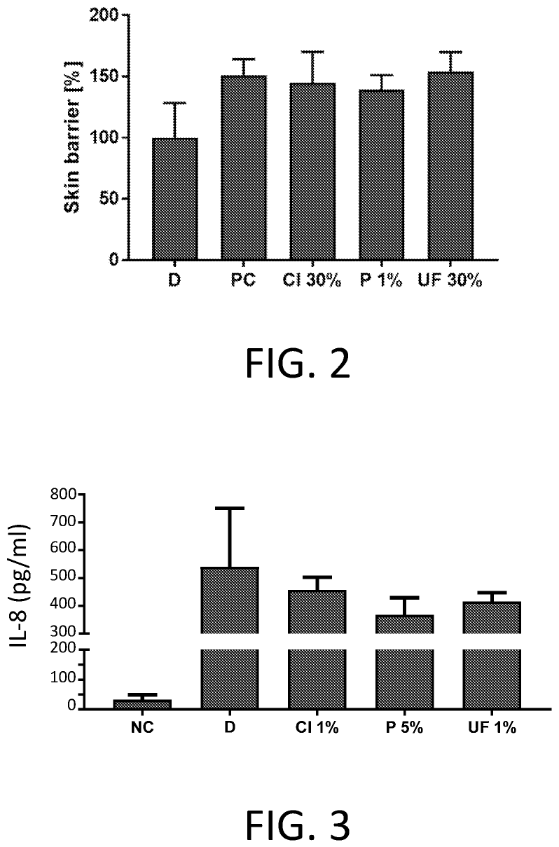

As shown in FIG. 2, application of the formulations containing each human milk fraction increased the TEER measurements observed in the in vitro human skin preparations. Formulations containing human milk cream or retentate displayed a trend of a dose-dependent increase in TEER (as measured in Ohm*cm² at 1,000 Hz) as compared to vehicle only control. Formulations containing 1% permeate increased TEER as compared to vehicle only control, although decreases in TEER were observed at higher concentrations of permeate. The observations of increased TEER are consistent with an increased barrier function following application of the human milk fractions.

No differences were observed in the media concentrations of IL-8, glucose, or LDH or in the MTT assay results following the application the human milk fractions to the skin preparations in any of the formulations. These results are consistent with an absence of adverse effects of the human milk fractions on skin inflammation, cellular metabolism, membrane integrity, and cellular viability. In agreement with these observations, no damage was observed in H&E stained cross sections of skin preparations following application of any of the formulations.

### Example 3: Effects of Human Milk Fractions on Three-Dimensional In Vitro Models of Skin Inflammation

Human milk fractions generated as described in Example 1 were evaluated for effects on skin inflammation. Three-dimensional in vitro skin preparations generated as described in Example 2 were incubated with 50 ng/ml TNF-alpha to model skin inflammation. After three hours of incubation, formulations containing the vehicle only control or human milk fractions similar to as described in Example 2 were applied to the in vitro skin preparations. An additional control that was untreated with TNF-alpha was also tested. After 24 hours, media was collected from the skin preparations and assessed for IL-8 levels by ELISA.

As shown in FIG. 3, a trend of reduced IL-8 was observed in media following treatments with formulations containing human milk cream, permeate, and retentate as compared to the vehicle only control. Treatment with formulations containing 5% permeate reduced IL-8 levels by approximately 30%. The reduced levels of IL-8 were consistent with anti-inflammatory effects of human milk fractions on human skin.

### Example 4: Effects of Human Milk Fractions on Three-Dimensional In Vitro Models of Damaged Skin

Human milk fractions generated as described in Example 1 were evaluated with three-dimensional in vitro models of damaged human skin.

In one experiment, three-dimensional in vitro skin preparations generated as described in Example 2 were mechanically disrupted with a friction stress resulting in reduced barrier function. Approximately twenty-four hours after mechanical disruption, formulations containing vehicle only (1% DMSO in PBS), a combination of human milk cream and permeate, or 30% skim milk in vehicle were applied to the in vitro skin preparations for 7 days. TEER was measured in the in vitro skin preparations similar to as described in Example 2 at approximately 3, 5, and 8 days after the mechanical disruption and was expressed as a percentage of TEER measurements collected from vehicle-only controls prior to the mechanical disruption.

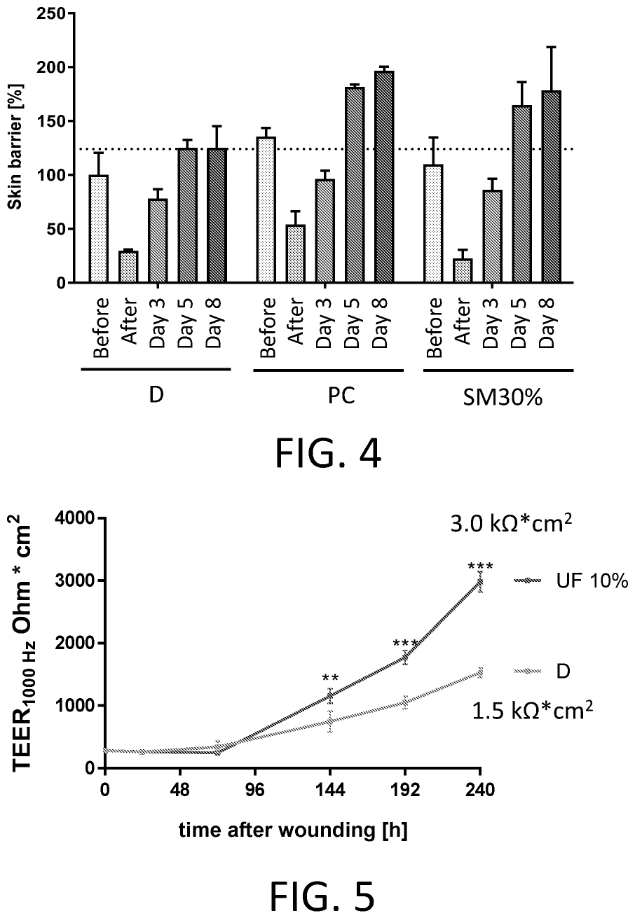

As shown in FIG. 4, increased TEER measurements were observed 5 and 8 days after the mechanical disruption in cream/permeate and skim milk treated skin preparations as compared to vehicle only controls. Since elevated TEER measurements indicate increased barrier function, these results are consistent with combined cream/permeate and skim milk fractions improving repair and barrier function in human skin.

In a second experiment, a dermal punch was removed from the in vitro skin preparations to evaluate re-epithelialization. Immediately after the dermal punch was collected, formulations containing vehicle only or human milk retentate were applied to the in vitro skin preparations. TEER was measured prior to and immediately after, and approximately 24 hours, 72 hours, 144 hours, 192 hours and 240 hours after the dermal punch was removed. As shown in FIG. 5, increased TEER measurements were observed in the in vitro skin preparations treated with formulations containing human milk retentate as compared to the vehicle only controls by the 144-hour time point, consistent with increased re-epithelialization and barrier function recovery.

### Example 5: Characterization of Human Milk Cream

The stability of human milk cream obtained as described in Example 1 was assessed by exposing samples of the cream to accelerated physical stress and heat stress. Rheological properties of human milk cream were also examined.

Response to accelerated physical stress was assessed by centrifuging samples of human milk cream at 13,000 rpm on a tabletop centrifuge for 2 minutes. The centrifugation separated the cream into three distinct layers: a clear bottom layer, a cream-colored middle layer, and a yellow liquid top layer.

Response to heat stress was assessed by storing samples of human milk cream at temperatures of 2-8° C., 25° C., 40° C., 50° C., or 70° C. Samples were visually inspected after 3 hours, 6 hours, and 24 hours of storage at each temperature. Phase separation was observed at all time points at 25° C. and above. A direct relationship between heat and the level of phase separation was observed. Sedimentation and color change were observed at 40° C. and above.

Rheology was assessed in samples containing either 100% human milk cream or 50% cream and 25% hydrocolloids. An oscillation stress sweep was performed to characterize the mechanical nature of the samples. Results indicated that the addition of hydrocolloids increased complex modulus and yield stress of the samples, consistent with a potential of hydrocolloids to reduce the probability of phase separation in formulations containing human milk cream. Rheology of samples containing 100% cream were also assessed with a frequency sweep. At low frequency, the loss modulus dominated the storage modulus, consistent with a relatively weak structure and high probability of phase separation of the cream. These observations were consistent with the cream having properties of a viscous solution and not a solid at rest.

Taken together, results from the stress tests and rheology assessment were consistent with human milk cream having a low density and a high probability of phase separation.

### Example 6: Generation of Hydrocolloid Systems Containing Human Milk Cream

Test hydrocolloid systems containing human milk cream obtained as described in Example 1 were formulated and assessed for stability. Hydrocolloid systems were prepared from various formulations containing between 50% and 75% (inclusive) human milk cream, one or both of xanthan gum and HPMC hydrocolloids, and one or more thickening agents selected from carabopol, carrageenan, HPC, paraffin wax, PEG 4000, sodium alginate, and Avacel CL-611. Samples prepared from each formulation were stored at 2-8° C., 25° C., 40° C., or 50° C. Visual and odor inspections were performed immediately after formulation (0 days) and after approximately two weeks of storage at each temperature. At 0 days, all of the hydrocolloid systems had a slight dairy odor. After two weeks, all formulations possessed a strong rancid odor regardless of physical appearance. Results of the visual inspections are summarized in Table E1.

TABLE E1
Summary of Visual Assessments of Hydrocolloid Systems

| Test | | 2 Weeks | | | |
|---|---|---|---|---|---|
| Formula | 0 Days | 2-8° C. | 25° C. | 40° C. | 50° C. |
| 1 | A | A | C | D | D |
| 2 | A | A | C | C | C |
| 3 | B | B | C | C | E |
| 4 | A | A | C | D | C |
| 5 | A | B | E | D | E |
| 6 | A | A | D | D | E |
| 7 | B | B | D | D | E |
| 8 | A | A | B | B | Not |
| 9 | B | A | C | D | Assessed |
| 10 | B | B | C | D | |
| 11 | A | A | D | D | |
| 12 | B | B | C | D | |

Macroscopic appearance
(A) = Pale yellow, low to medium viscosity, pourable, smooth application
(B) = Pale yellow, not easily pourable, medium to high viscosity
(C) = Slight phase separation (2 layers), fats on top layer, medium to high viscosity
(D) = Phase separation (2 layers): fatty yellow top layer, pale yellow bottom layer
(E) = Phase separation (3 layers)

All of the formulations appeared to improve stability as compared to human milk cream alone. Test formula 8 appeared to be the most stabile formulation, as no phase separation was observed at any temperature tested, although viscosity increased after 2 weeks at 25° C. and 40° C. None of the other formulations prevented phase separation from occurring after two weeks of storage at temperatures of 25° C. or greater.

These results are consistent with a compatibility of human skin cream to be formulated into a stable hydrocolloid system, with the degree of the stability depending on the specific formulations.

### Example 7: Generation and Characterization of Different Compositions Containing Human Milk Cream

Human milk cream was formulated into skin cream, hydrocolloid system, emulsified gel, and oleaginous ointment compositions. Samples from each composition were evaluated shortly after formulation (0 days) and after two and four weeks of storage at 2-8° C., 25° C., 40° C., or 50° C. At each time point, samples were assessed for apparent pH, macroscopic and microscopic appearance, and rheology. Rheology was assessed similar to as described in Example 6. For all test compositions, an increase in the complex modulus and apparent yield stress was observed following two weeks at 50° C., and so rheology was not assessed at four-week time points for 50° C. conditions.

No rancid odors were detected following storage after 2 weeks at any temperature or at 25° C. and 40° C. after 4 weeks in any of the compositions prepared from any of the formulations. The apparent pH was measured between 6.6 and 6.9 for the hydrocolloid system, emulsified gel, and oleaginous ointment compositions, and between 4.4 and 4.7 in the cream preparations. The pH values did not appear to change over time in any of the compositions.

### A) Skin Cream Preparations

Test skin cream preparations containing either 5% or 20% human milk cream obtained as described in Example 1 were formulated from aqueous and oil phases. The aqueous phases were composed of xanthan gum, cetostearyl alcohol, and the presence or absence of phenoxyethanol, benzyl alcohol, and steric acid. The lipid phases contained white soft paraffin and the presence or absence of liquid paraffin, cetomacrogol 1000, Tween 60, span 60, Brij S2 (Brij 72), Brij S721 (Brij 721), ascorbyl palmitate, and ascorbic acid. Formulations also contained the presence or absence of anti-oxidants.

At 0 days, the skin cream preparations had a white color, with one exception having a pale-yellow color that turned to an off-white color following 4 weeks of storage at 25° C. or 40° C. The skin cream preparations had a high viscosity and were not pourable. No rancid odors were detected in cream preparations from any of the formulations following 2 weeks or at 25° C. and 40° C. after 4 weeks. Aeration was observed following 2 weeks in all of the cream preparations at all temperatures tested.

Small homologous oil droplets could be observed under the microscope at 0 days in all of cream preparations. An increase in droplet size was observed following storage at 50° C. for two weeks although no observable increase was observed in any of the preparations at lower temperatures. Only one of the skin cream formulations underwent phase separation which occurred after two weeks at 50° C.

Measured values of the complex modulus and apparent yield stress of the cream preparations were similar at 0 days and at 4 weeks following storage at 25° C. and 40° C.

### B) Hydrocolloid Systems

Test hydrocolloid systems were prepared from formulations containing 50% human milk cream, one or more hydrocolloids selected from xanthan gum, sodium alginate, Protanal HF120PBS, and Avicel CL-611. Benzyl alcohol was added as a preservative, and ascorbyl palmitate and alpha-tocopherol were added as antioxidants to each formulation. Samples prepared from each formulation were stored at 2-8° C., 25° C., 40° C., or 50° C. The hydrocolloid systems were assessed for pH, visual appearance, and odor after 0 days, approximately 2 weeks, and approximately four weeks of storage at each temperature.

At 0 days, the hydrocolloid systems had a pale-yellow color. Orange particles appeared in the hydrocolloid systems after 2 weeks at 50° C. and after 4 weeks at 25° C. and 40° C. In half of the formulations tested, viscosity increased after 4 weeks of storage at 25° C. and 40° C.

Non-homologous oil droplets were observed with a microscope in hydrocolloid systems at 0 days and following 2 and 4 weeks storage at 25° C., 40° C., and 50° C. No excipient crystals were observed in any condition.

Rheological measurements collected at 0 days, 2 weeks, and 4 weeks from the test hydrocolloid systems were consistent with a greater instability among hydrocolloid systems as compared to other cream preparations and emulsified gels. The complex modulus of the test hydrocolloid systems increased by two weeks in storage at 40° C. or 50° C. However, some of the formulations maintained similar values for the complex modulus over two and four weeks storage at 25° C.

### C) Emulsified Gel Compositions

Emulsified gel compositions were produced from formulations containing either 5% or 20% human milk cream, an aqueous phase, and an oil phase. The aqueous phase included polysorbate 80 and benzyl alcohol. The oil phase included the cream, liquid paraffin, and Span 83. The formulations also included antioxidants and xanthan gum as a gelling agent.

At 0 days, the emulsified gels had a slight yellow color, low viscosity, and were pourable. A medium viscosity and slow pourability were observed following storage for two weeks at 40° C. and four weeks at 25° C. A high viscosity and a lack of pourability were observed following storage for two weeks at 50° C. and four weeks at 40° C. A slight translucent layer was observed in the emulsified gel containing 20% cream following storage for weeks at 40° C.

As typical for emulsified gel compositions, small oil droplets were observed under a microscope at 0 days and following storage for two weeks at 40° C. and 50° C. and four weeks at 25° C. and 40° C.

Rheological measurements indicated that, among the different types of compositions tested, the emulsified gels displayed the least changes in mechanical behavior during storage.

### D) Oleaginous Ointments

Oleaginous ointment compositions were produced from formulations containing either 5% or 20% human milk cream. The excipients of each formulation included liquid paraffin, IPM, crodamol GRCC, castor oil, whit soft paraffin, cetomacrogol 1000, and cetostearyl alcohol.

At 0 days, the oleaginous ointment composition containing 5% human milk cream had an off-white color. The composition containing 20% cream appeared yellow. The oleaginous ointment compositions initially possessed a medium viscosity and were not pourable. An increase in viscosity was observed following storage for two and four weeks under all temperature conditions.

Large solid fluorescent particles (potentially due to cetostearyl alcohol excipient) were observed at 0 days and following storage for 2 and 4 weeks at all temperatures.

Measurements of complex modulus and apparent yield stress from the oleaginous ointments were variable at all time points and at all temperatures examined, consistent with a greater instability of oleaginous ointments as compared to the other test compositions.

### Example 8: Generation of Nanoemulsions Containing Human Milk Cream

Human milk cream that was generated similar to as described in Example 1 was formulated into different nanoemulsion formulations. Nanoemulsions from 27 different test formulations containing either 10%, 20%, or 30% human milk cream by weight and different combinations of excipients were prepared. The nanoemulsions were prepared in 1 mL volumes with either one or two rounds of highspeed bead-beading followed by centrifugation to remove foam. The resulting nanoemulsions were evaluated for appearance and clarity. Translucent and uniform samples were inspected by microscope for the presence of oil droplets. Samples were placed on a rotator at room temperature for 2-3 days and inspected for appearance and particle size via laser diffraction analysis to identify nanoemulsions that remained translucent and uniform.

Based on the results of the initial testing, four test formulations were selected for further evaluation. Forty grams of nanoemulsion for each of the four test formulations were produced. The formulations included 10% human milk cream by weight, in solution containing EDTA and glycerin. The formulations also contained various amounts of soy lecithin and the presence or absence of histidine and benzalkonium chloride. Three of the formulations were then pH adjusted to 7.5±0.5 and the fourth was adjusted to 11.5±0.5. The formulations were processed with a Microfluidizer, and the resulting nanoemulsions were each pH adjusted to 7.0±0.5 and filtered through a 0.22 μm membrane into a sterile container. The filtrates were stored at 2-8° C., 25° C., or 40° C. and inspected for appearance and particle size via laser diffraction analysis twice weekly. Results are summarized in Tables E1-E4 below.

TABLE E1
Appearance and Particle Size of Nano-Emulations at Day 0

| | Test | Test | Test | Test |
|---|---|---|---|---|
| | Formula 1 | Formula 2 | Formula 3 | Formula 4 |
| Appearance | Off-white/ | Off-white/ | Off-white/ | Off-white/ |
| | Translucent | Translucent | Translucent | Translucent |
| Z-Avg (nm) | 97 | 93 | 96 | 94 |

TABLE E2
Appearance and Particle Size of Nano-Emulations at 2-8° C.

| | | Test | Test | Test | Test |
|---|---|---|---|---|---|
| | Day | Formula 1 | Formula 2 | Formula 3 | Formula 4 |
| Appearance | 3 | Off-white/ | Off-white/ | Off-white/ | Off-white/ |
| | | Opaque | Opaque | Opaque | Opaque |
| Z-Avg (nm) | | 124 | 104 | 110 | 139 |
| Appearance | 6 | Off-white/ | Off-white/ | Off-white/ | Off-white/ |
| | | Opaque | Opaque | Opaque | Opaque |
| Z-Avg (nm) | | 144 | 111 | 118 | 147 |
| Appearance | 10 | Off-white/ | Off-white/ | Off-white/ | Off-white/ |
| | | Opaque | Opaque | Opaque | Opaque |
| Z-Avg (nm) | | 135 | 117 | 126 | 156 |
| Appearance | 16 | Off-white/ | Off-white/ | Off-white/ | Off-white/ |
| | | Opaque | Opaque | Opaque | Opaque |
| Z-Avg (nm) | | 131 | 121 | 126 | 155 |

TABLE E3
Appearance and Particle Size of Nano-Emulations at 25° C.

| | | Test | Test | Test | Test |
|---|---|---|---|---|---|
| | Day | Formula 1 | Formula 2 | Formula 3 | Formula 4 |
| Appearance | 3 | Off-white/ | Off-white/ | Off-white/ | Off-white/ |
| | | Trans- | Trans- | Trans- | Trans- |
| | | lucent | lucent | lucent | lucent |
| Z-Avg (nm) | | 125 | 109 | 117 | 139 |
| Appearance | 6 | Off-white/ | Off-white/ | Off-white/ | Off-white/ |
| | | Trans- | Trans- | Trans- | Opaque |
| | | lucent | lucent | lucent | |
| Z-Avg (nm) | | 129 | 112 | 118 | 143 |
| Appearance | 10 | Off-white/ | Off-white/ | Off-white/ | Off-white/ |
| | | Trans- | Trans- | Trans- | Opaque |
| | | lucent | lucent | lucent | |
| Z-Avg (nm) | | 140 | 117 | 123 | 151 |
| Appearance | 16 | Off-white/ | Off-white/ | Off-white/ | Off-white/ |
| | | Opaque | Trans- | Trans- | Opaque |
| | | | lucent | lucent | |
| Z-Avg (nm) | | 151 | 121 | 124 | 146 |

TABLE E4
Appearance and Particle Size of Nano-Emulations at 40° C.

| | | Test | Test | Test | Test |
|---|---|---|---|---|---|
| | Day | Formula 1 | Formula 2 | Formula 3 | Formula 4 |
| Appearance | 3 | Off-white/ | Off-white/ | Off-white/ | Off-white/ |
| | | Trans- | Trans- | Trans- | Opaque |
| | | lucent | lucent | lucent | |
| Z-Avg (nm) | | 157 | 130 | 139 | 179 |
| Appearance | 6 | Off-white/ | Off-white/ | Off-white/ | Off-white/ |
| | | Trans- | Trans- | Trans- | Opaque |
| | | lucent | lucent | lucent | |
| Z-Avg (nm) | | 159 | 135 | 147 | 174 |
| Appearance | 10 | Off-white/ | Off-white/ | Off-white/ | Off-white/ |
| | | Trans- | Trans- | Trans- | Opaque |
| | | lucent | lucent | lucent | |
| Z-Avg (nm) | | 168 | 139 | 155 | 190 |
| Appearance | 16 | Off-white/ | Off-white/ | Off-white/ | Off-white/ |
| | | Opaque | Trans- | Trans- | Opaque |
| | | | lucent | lucent | |
| Z-Avg (nm) | | 272 | 142 | 157 | 184 |

Translucent samples appeared clear under a microscope, while small particles could be detected in opaque samples. The nanoemulsions appeared uniform at all time points and temperatures examined and the average globule size was stable over time. Taken together, these observations are consistent with production of stable nanoemulsions containing human milk cream.

### Example 9: Generation of Oil-In-Water Emulsions Containing Human Milk Cream

Various oil-in-water emulsions were generated containing one or both of human milk cream and permeate fractions produced as described in Example 1. The emulsions were produced by mixing various aqueous and oil phases for use as test emulsions. Aqueous phases included those formulated by combining water or permeate in the absence or presence of various levels of human milk cream, Tween 80, and phenoxyethanol. The oil phases were formulated with liquid paraffin and Span 83. Alpha tocopherol was added as an antioxidant, and xanthene gum was added as a thickening agent.

The oil-in-water emulsions were analyzed with a particle size analyzer (Malvern 2000, Malvern Panalytical, Malvern, U.K.). Emulsions containing Tween 80 had lower mean particle size than comparable emulsions lacking Tween 80. In addition, it was observed that viscosity of the emulsions increased with Tween 80 concentration. For emulsions containing human milk cream, those formulated with homogenized human milk cream generally had narrower particle size distributions than similar emulsions formulated with non-homogenized cream.

### Example 10: Effects of Oil-In-Water Emulsions Containing Human Milk Cream and Permeate on TNF-Induced IL-8 Secretion in an In Vitro Model of Human Skin

Oil-in-water emulsions containing one or both of the human milk cream and permeate fractions produced as described in Example 1 were generated as test emulsions. The test emulsions were assessed on an in vitro model of human skin similar to as described in Example 2. The test emulsions were generated by combining (i) aqueous phases produced by combining water or permeate with Tween 80 and phenoxyethanol with or without human milk cream with (ii) oil phases formulated with liquid paraffin and Span 83. Alpha tocopherol was added as an antioxidant, and xanthene gum was added as a thickening agent. An emulsion that lacked human milk permeate and cream served as a vehicle control, and a commercially available topical formulation served as a positive control.

The test emulsions and controls were applied to the in vitro human skin models. TNF (long/ml) was applied to the human skin models in parallel with additional experimental controls of untreated skin models incubated with or without TNF. The human skin models were incubated in the presence of the control and test emulsions for 72 hours, followed by a 48 hour post treatment incubation in the absence of the control and test emulsions. The human skin models were evaluated for IL-8 secretion at 24 and 72 hour time points and after the 48 hour incubation by solid-phase sandwich ELISA.

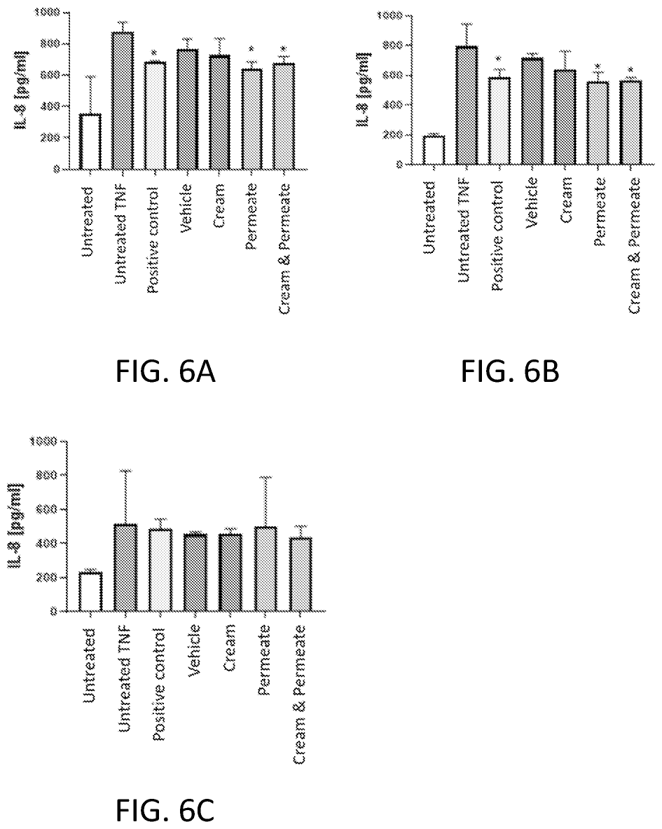

As shown in FIG. 6A-C, TNF exposure was associated with increased levels of extracellular IL-8. Application of test emulsions containing human milk permeate or both human milk permeate and cream resulted in a significant reduction of the TNF-stimulated IL-8 secretion at 24 hour (FIG. 6A) and 72 hour (FIG. 6B) time points. No effects on TNF stimulated IL-8 secretion were observed following the 48 hour post-treatment incubation (FIG. 6C). These results are consistent with anti-inflammatory properties of human milk permeate and human milk cream.

### Example 11: Generation of an Oil-In-Water Emulsion Containing Human Cream and Permeate Fractions

An oil-in-water emulsion was produced containing human cream and human milk permeate fractions that were generated similar to as described in Example 1. The cream fraction was pressure homogenized using a Lactoscope FT-A (Perkin Elmer) and the total fat content of the cream was measured after homogenization. The emulsion was formulated by first gently combining separate aqueous phase and oil phase mixtures, adding the homogenized cream, and then adding the xanthene gum as a thickening agent. The aqueous phase was formulated from Tween 80, phenoxyethanol, and a permeate fraction containing at least 0.5% HMO w/v. The oil phase contained liquid paraffin, Span 83, and alpha tocopherol. The homogenized cream was added in an amount sufficient for the final emulsion to contain 10% human milk fat (w/v).

Samples from the resulting oil-in-water emulsion were analyzed with a particle size analyzer (Malvern 2000, Malvern Panalytical, Malvern, U.K.). The mean particle size of the emulsion was determined to be between 0.5 and 1.0 microns in diameter.

Anti-microbial efficacy tests (AET) were performed on samples of the emulsion. The samples were stored in refrigerator for 4 weeks before AET testing. Controlled inoculums of challenge organisms that included S. aureus, P. aeruginosa, E. coli, C. albicans, and A. brasiliensis were placed in suspension with samples of the emulsion and counts of surviving challenge organisms were assessed at baseline (day 0) and days 7, 14, and 28. At day 7, 14, and 28 time points, counts were at or below the threshold of detection and consistent with a preservative activity of the emulsion capable of preventing microbial growth.

Fatty acid profiles of samples collected from the emulsion were generated by gas chromatography-mass spectrometry (GC-MS). Samples were collected at day 0 and at various timepoints after formulation. Fatty acid profiles were generated by calculating the value of the percentage by weight of individual fatty acid species as compared to total fatty acid content. Comparisons of fatty acid profiles were performed by determining the correlation between measurements of the individual fatty acid species for each profile. A comparison of the fatty acid profiles measured from samples of the same manufacturing lot that were stored between 2° C. and 8° C. or at room temperature and collected at day 0 and at 3-month, 6-month, 9-month, and 12-month time points indicated little change in the fatty acid profile between these time points from day 0, consistent with a stability of the emulsion for at least 1 year when refrigerated or stored at room temperature.

### Example 12: Treatment of Psoriasis with a Topical Formulation Containing Human Milk Ingredients

The safety and tolerability of a topical formulation containing human milk ingredients that is formulated similar to as described in Example 11 is assessed for the treatment of psoriasis in an open label, single-site study. The study enrolls male and female subjects aged 18 or older who meet criteria including: i) a clinical diagnosis of chronic plaque psoriasis and stable disease for at least 6 months prior to the study; and ii) body surface area involvement of 2% to 20% (excluding scalp, palms, fingernails, toenails, and soles).

The subjects apply the topical formulation to affected areas twice daily approximately 12 hours apart for 8 weeks. The ingredients of the topical formulation are summarized in Table E5.

TABLE E5
ingredient list

| Ingredient | Concentration (% w/w) |
|---|---|
| Polysorbate 80 | ≤2 |
| Phenoxyethanol | ≤2 |
| Liquid Paraffin | ≤10 |
| Sorbitan Monooleate | ≤2 |
| Xanthan Gum | ≤3 |
| Alpha-Tocopherol | ≤0.005 ml/100 g |
| Human milk components | ≥80 (10% fat) |

Lesions will be photographed at baseline (day 1) and 2, 4, and 8 weeks. Assessments of the treatment may include peak pruritus numeric rating scale and surveys to assess tolerability and satisfaction with the treatment. Safety assessment will be performed for incidence, frequency, and duration of any treatment related adverse events (TRAEs), serious adverse events (SAEs), or adverse events (AEs) leading to study discontinuation.

Fewer severe and/or total lesions or affected areas as compared to baseline may be observed during treatment. Additional observations may be consistent with reduced psoriasis severity, reduced skin irritation or itching sensation, and/or improved skin appearance during the treatment as compared to baseline. Results may also indicate that the topical formulations are well tolerated. A low incidence, frequency, and/or duration of TRAEs, SAEs, and/or AEs, if any, will be observed during treatment.

### Example 13: Treatment of Psoriasis with a Topical Formulation Containing Human Milk Ingredients

A clinical study assessing safety and tolerability of a topical formulation for treatment of psoriasis was performed similar to as described in Example 12. The topical formulation was produced similar to as described in Example 11 and contained the ingredients summarized in Table E5. Eleven subjects completed the study.

The topical formulation was generally well tolerated by patients. The subjects maintained a patient tolerability score of “0” throughout the study, with the exception of one subject who on week 2 reported a tolerability score of “1”, which was defined as “slight—an awareness, but no discomfort and no intervention required.” This subject reported a score of “0” for all subsequent visits. No evidence of irritation or intolerability of the topical formulation was observed by the investigator assessment at weeks 2, 4, and 8. No adverse events or serious adverse events were reported.

Subjects assessed itch with a ten-point Peak Pruritus Numerical Rating Scale, with higher numbers indicating more severe itch sensation. The mean Peak Pruritus scores are shown in Table E6.

TABLE E6
Mean Peak Pruritus Assessments

| Visit | Mean Score |
|---|---|
| Baseline | 4.1 |
| Week 2 | 2.4 |
| Week 4 | 2.6 |
| Week 8 | 2.9 |

Five of the subjects reported scores lower than baseline during the course of the study, and only 1 subject reported an increases score at week 8 relative to baseline. Review of the sequential photographs revealed a trend toward improvement of the underlying disease state psoriasis as evidenced by reduced erythema, scale, desquamation and lichenification.

Taken together, these data are consistent with human milk derived topical formulations as a safe remedy for the symptoms of plaque psoriasis.

### Example 14: Treatment of Atopic Dermatitis with a Topical Formulation Containing Human Milk Ingredients

The safety and tolerability of two topical formulations containing human milk ingredients are assessed for the treatment of psoriasis in an open label, single-site study. The two topical formulations were formulated similar to as described in Example 11, with one of the formulations having 10% w/w human milk fat and the other having 20% w/w human milk fat. The ingredients are summarized in Table E7.

TABLE E7
ingredient list

| Ingredient | Concentration (% w/w) |
|---|---|
| Polysorbate 80 | ≤2 |
| Phenoxyethanol | ≤2 |
| Liquid Paraffin | ≤10 |
| Sorbitan Monooleate | ≤2 |
| Xanthan Gum | ≤3 |
| Alpha-Tocopherol | ≤0.005 ml/100 g |
| Human milk components | ≥80 (10% or 20% fat) |

The study enrolls male and female subjects aged 18 or older who meet criteria including: i) a clinical diagnosis of chronic atopic dermatitis and stable disease for at least 6 months prior to the study; and ii) body surface area involvement of less than or equal to 35% (excluding scalp, palms, fingernails, toenails, and soles).

The subjects will each apply one of the topical formulation to affected areas twice daily approximately 12 hours apart for 8 weeks.

Lesions will be photographed at baseline (day 1) and 2, 4, and 8 weeks. Assessments of the treatment may include surveys to assess tolerability and satisfaction with the treatment and investigator assessments of the presence and overall degree of irritation at the application sites e.g., according to a 5-point scale such as the one depicted by Table E7.

TABLE E8
Local Tolerability

| Score | Severity | Description |
|---|---|---|
| 0 | None | Clear skin - Minor residual hypo/hyperpigmentation; |
| | | no erythema or induration/papulation: no oozing/ |
| | | crusting |
| 1 | Mild | Almost clear - Trace faint pink erythema, with barely |
| | | perceptible induration/papulation and no oozing/ |
| | | crusting |
| 2 | Moderate | Mild severity - Faint pink erythema with mild |
| | | induration/papulation and no oozing/crusting |
| 3 | Severe | Moderate severity - Pink-red erythema with moderate |
| | | induration/papulation with or without oozing/crusting |
| 4 | Very | Severe- Deep or bright red erythema with severe |
| | Severe | induration/papulation and with oozing/crusting |

Safety assessment will be performed for incidence, frequency, and duration of any or adverse events (AEs), serious adverse events (SAEs), or any adverse events leading to study discontinuation.

Fewer severe and/or total lesions or affected areas as compared to baseline may be observed during treatment. Additional observations may be consistent with reduced psoriasis severity, reduced skin irritation or itching sensation, and/or improved skin appearance during the treatment as compared to baseline. Results may also indicate that the topical formulations are well tolerated, with a low incidence, frequency, and/or duration of SAEs and/or AEs, if any, observed during treatment.

### Example 15: Treatment of Atopic Dermatitis with a Topical Formulation Containing Human Milk Ingredients

The safety and tolerability of topical formulations containing cream from human milk was evaluated for the treatment of atopic dermatitis in a clinical study that was performed similar to as described in Example 12. The topical formulation was produced similar to as described in Example 11 and contained the ingredients summarized in Table E7. The first six subjects who enrolled received the topical formulation containing 10% human milk fat, while the remaining eight subjects received to topical formulation with 20% human milk fat.

The topical formulations were generally well tolerated by subjects. None of the subjects reported irritation or irritability due to either of the topical formulations and no evidence of irritation or irritability due to the topical formulations was observed in subjects at any of the 2, 4, or 8 week study visits.

Subjects assessed itch with a ten-point Peak Pruritus Numerical Rating Scale, with higher numbers indicating more severe itch sensation. The mean Peak Pruritus scores for subjects who received topical formulations containing 10% or 20% human milk fat are shown in Table E9. A trend of reduced peak pruritus scores was observed across the 8 week treatment in both study groups.

TABLE E9
Mean Peak Pruritus Assessments

| Visit | 10% Fat | 20% Fat |
|---|---|---|
| Baseline | 5.0 | 5.3 |
| Week 2 | 4.3 | 4.5 |
| Week 4 | 3.6 | 5.6 |
| Week 8 | 3.8 | 3.3 |

Taken together, these data are consistent with human milk derived topical formulations as a safe remedy for the symptoms of atopic dermatitis.

### Example 16: Generation of a Topical Formulation Containing Human Milk Ingredients

A topical formulation was produced that contained human milk cream and human milk permeate fractions that were generated similar to as described in Example 1. The total fat content of the cream was measured with a Lactoscope FT-A (Perkin Elmer). Components of the topical formulation is shown are Table E10. A topical formulation containing 20% fat and the human milk permeate with Tween 80, phenoxyethanol, liquid paraffin, Span 83, and alpha tocopherol and mixing with an IKA T25 Dispersing unit at 25000 rpm for about 30 minutes at room temperature. The final topical formulation was produced by slowly adding Xanthan gum and mixing at 700 rpm for about 15 minutes.

TABLE E10
Ingredients of Topical Formulation

| Ingredient | Concentration (% w/w) |
|---|---|
| Polysorbate 80 | ≤6 |
| Phenoxyethanol | ≤1 |
| Liquid Paraffin/Humectant(Glycerin) | ≤10 |
| Sorbitan Monooleate | ≤3 |
| Xanthan Gum | ≤2 |
| Alpha-Tocopherol | ≤0.005 ml/100 g |
| Human milk components | ≥78 (10% or 20% fat) |
| (cream and permeate) | |

### Example 17: Generation of Human Milk Cream Sub-Fractions

Human milk cream was separated from whole human milk as described in Example 1. The resulting human milk cream fraction was then homogenized at 20,000 psi at a rate of 60 ml/min for one passage. The homogenized human milk cream was then centrifuged at 3,500 g for 30 minutes at ambient temperature. The homogenized cream separated into three subfractions: a top layer composed of butteroil, a middle layer composed of milk fat globule membrane solids, and a bottom layer containing skim milk. Topical formulations containing 20% fat (w/w) were produced from the butteroil and milk fat globule membrane solids sub-fractions by substituting the human milk cream with the butteroil or milk fat globule membrane solids sub-fractions in the procedure described in Example 16.

Additional topical formulations are produced by substituting human milk cream with the butteroil or the milk fat globule membrane solids sub-fractions in any of the methods, processes, or recipes for generating topical formulations described in Examples 6-9, 11, 12, or 14.

1. A topical formulation comprising one or more human milk fractions and one or more pharmaceutically acceptable excipients,
wherein the one or more human milk factions comprise one or more of human milk cream or a sub-fraction thereof, human skim milk, human milk permeate, or human milk retentate, and wherein the human milk permeate and the human milk retentate are obtained from filtration of human skim milk.

2. The topical formulation of claim 1, wherein the topical formulation comprises human milk cream or a sub-fraction thereof.

3. The topical formulation of claim 1 or 2, wherein the topical cream comprises human milk butteroil and/or human milk globule membrane solids.

4. The topical formulation of any of claims 1-3, wherein the topical formulation comprises human milk fatty acids.

5. The topical formulation of any of claims 1-4, wherein the topical formulation comprises one or more of oleic acid, palmitic acid, linoleic acid, omega 3 fatty acid, omega 6 fatty acid, omega 9 fatty acid, stearic acid, myristic acid, lauric acid, palmitoleic acid, vaccenic acid, myristolic acid, heptadecenoic acid, gamma linolenic acid, docosapentaenoic acid, eicosapentaenoic acid, arachidonic acid, and docosahexaenoic acid.

6. The topical formulation of any of claims 1-5, wherein the topical formulation comprises oleic acid, palmitic acid, linoleic acid, omega 3 fatty acid, omega 6 fatty acid, omega 9 fatty acid, stearic acid, myristic acid, lauric acid, palmitoleic acid, vaccenic acid, myristolic acid, heptadecenoic acid, gamma linolenic acid, docosapentaenoic acid, eicosapentaenoic acid, arachidonic acid, and docosahexaenoic acid.

7. The topical formulation of any of claims 1-6, wherein the topical formulation comprises at least 1% human milk fat (weight/weight).

8. The topical formulation of any of claims 1-7, wherein the topical formulation comprises from 1% to 40% or 10% to 30% human milk fat (weight/weight).

9. The topical formulation of any of claims 1-8, wherein the topical formulation comprises about 10% human milk fat (weight/weight).

10. The topical formulation of any of claims 1-8, wherein the topical formulation comprises about 20% human milk fat (weight/weight).

11. The topical formulation of any of claims 1-8, wherein the topical formulation comprises about 30% human milk fat (weight/weight).

12. The topical formulation of any of claims 1-11, wherein the topical formulation comprises human milk permeate, wherein the human milk permeate is obtained from filtration of human skim milk.

13. The topical formulation of claim 12, wherein the filtration comprises ultra-filtration.

14. The topical formulation of any of claims 1-13, wherein the topical formulation comprises at least one human milk oligosaccharide.

15. The topical formulation of any of claims 1-14, wherein the topical formulation comprises at least 2, 5, 10, 25, 50, or more different human milk oligosaccharides.

16. The topical formulation of any of claims 1-15, wherein the topical formulation comprises at least 0.1% human milk oligosaccharides (weight/weight).

17. The topical formulation of any of claims 1-16, wherein the topical formulation comprises at least 0.5% human milk oligosaccharides (weight/weight).

18. The topical formulation of any of claims 1-16, wherein the topical formulation comprises from 0.1% to 1.0% human milk oligosaccharides (weight/weight).

19. The topical formulation of claim 18, wherein the topical formulation comprises about 0.3% human milk oligosaccharides (weight/weight).

20. The topical formulation of any of claims 1-19, wherein the topical formulation comprises one or more of 2′-fucosyl-lactose, 3-fucosyl-lactose, 3′-sialyl-lactose, 6′-sialyl-lactose, lacto-N-tetraose, lacto-N-difucohexaose I, lactodifucotetraose, lacto-N-fucopentaose I, sialylacto-N-tetraose c, sialylacto-N-tetraose b, and disialyllacto-N-tetraose.

21. The topical formulation of any of claims 1-20, wherein the topical formulation comprises 2′-fucosyl-lactose, 3-fucosyl-lactose, 3′-sialyl-lactose, 6′-sialyl-lactose, lacto-N-tetraose, lacto-N-difucohexaose I, lactodifucotetraose, lacto-N-fucopentaose I, sialylacto-N-tetraose c, sialylacto-N-tetraose b, and disialyllacto-N-tetraose.

22. The topical formulation of any of claims 1-21, comprising human milk retentate, wherein the human milk retentate is obtained from filtration of human skim milk.

23. The topical formulation of claim 22, wherein the filtration is or comprises ultra-filtration.

24. The topical formulation of any of claims 1-23, comprising one or more human milk proteins.

25. The topical formulation of any of claims 1-24, comprising one or more of casein, α-lactalbumin, lactoferrin, secretory immunoglobulin IgA, lysozyme, serum albumin, lactadherin, dermcidin, immunoglobulins, cytokines, mucins, or growth factors,

26. The topical formulation of any of claim 1-7 or 12-25, wherein the topical formulation comprises at least 0.1% human milk oligosaccharides (weight/weight) and between 1% and 40% human milk fat (weight/weight).

27. The topical formulation of claim 26, wherein the topical formulation comprises about 10% human milk fat (weight/weight).

28. The topical formulation of claim 25, wherein the topical formulation comprises about 20% human milk fat (weight/weight).

29. The topical formulation of claim 25, wherein the topical formulation comprises about 30% human milk fat (weight/weight).

30. The topical formulation of any of claims 1-29, wherein the one or more excipients comprise one or more of a hydrocolloid, a thickening agent, an antioxidant, a preservative, or a gelling agent.

31. The topical formulation of any of claims 1-30, wherein the topical formulation is or comprises an oil-in water emulsion, a water-in-oil emulsion, a nanoemulsion, an emulsified gel, an oleaginous ointment, a hydrocolloid system, or a cream preparation.

32. The topical formulation of any of claims 1-31, wherein the topical formulation is or comprises an oil-in-water emulsion.

33. A method of treatment, comprising administering the topical formulation of any of claims 1-32 to a subject in need thereof.

34. The method of claim 33, wherein the subject displays or is at risk of displaying one or more symptoms associated with a skin condition or disorder.

35. The method of claim 34, wherein administering the topical formulation—comprises applying the topical formulation directly to a region of skin displaying or at risk of displaying the one or more symptoms.

36. The method of claim 34 or 35, wherein the skin condition or disorder comprises one or more of dermatitis, atopic dermatitis, contact dermatitis, hand dermatitis, allergic dermatitis, gravitational dermatitis, asteatotic dermatitis, nummular dermatitis, seborrhoeic dermatitis, infective dermatitis, acne, acne vulgaris, rosacea, epidermolysis bullosa simplex, chronic superficial scaly acrodermatitis, chondrodermatitis, perioral dermatitis, dermatomyositis, neutrophilic dermatosis, transient acantholytic dermatosis, eczema, ichthyosis, xerosis, asteatosis, psoriasis, lupus erythematosus, exfoliative keratolysis, impetigo, allergic inflammation of the skin, urticaria, laceration, burn, or a wound resulting from a surgical incision or a biopsy.

37. The method of any of claims 34-36, wherein the skin condition or disorder comprises atopic dermatitis.

38. The method of any of claims 34-37, wherein the one or more symptoms associated with the skin condition or disorder comprise one or more of dryness, lesions, cracks, fissures, scaling, discoloration, thickening, redness, bleeding, swelling, flaking, ulcers, sores, wounds, blistering, rashes, inflammation, infection, or loss of flesh.

39. The method of any of claims 34-38, wherein the administering the topical formulation comprises directly applying the topical formulation to a region of skin having or at risk of having the one or more symptoms.

40. The method of any of claims 34-39, wherein the one or more symptoms comprises inflammation.

41. A method for reducing or preventing inflammation in a subject in need thereof, comprising applying the topical formulation of any of claims 1-32 directly to a region on the subject's skin or in the subject's mouth that is or is at risk of being inflamed.

42. A method of treating a wound, comprising applying the topical formulation of any of claims 1-32 to a subject in need thereof, wherein the topical formulation is applied directly to the wound, optionally to promote healing or reduce probability or degree of scarring.

43. The method of any of claims 33-42, wherein the subject is or is at risk of experiencing pain associated with breastfeeding.

44. The method of claim 43, wherein the administration comprises applying the topical formulation to the subject's breast or nipple.

45. A method of reducing transepidermal water loss in a subject in need thereof, comprising applying the topical formulation of any of claims 1-32 directly to a region on the subject's skin.

46. A method for treating mucositis, comprising administering the topical formulation of any of claims 1-32, to a subject in need thereof, wherein the topical formulation is administered orally and/or topically to the oral mucosa.

47. The method of claim 46, wherein the one or more symptoms associated with mucositis comprises one or more of pain, inflammation, or ulceration.

48. The method of claim 46 or 47, wherein the topical formulation is applied to an oral lesion.

49. A method for improving the appearance of aged or damaged skin, comprising administering the topical formulation of any of claims 1-32 to a subject in need thereof.

50. The method of claim 49, wherein administering the topical formulation comprises directly applying the topical formulation to the aged or damaged skin.

51. The method of claim 49 or 50, wherein the aged or damaged skin comprises one or more of inflammation, irritation, and erythema of the skin, wrinkles, skin blemishes, “marionette” lines, smile lines, deep nasolabial fold lines, crow's feet, fine lines, vertical lines between the eyebrows, horizontal forehead lines, sagging thin/frail skin, dryness, dullness, loss of elasticity, lack of radiance, appearance of spider vessels or red blotchiness, exaggerated lines, and wrinkles.

52. The topical formulation of any of claims 1-32 for use in the treatment or prevention of one or more symptoms associated with a skin condition or disorder.

53. The topical formulation of any of claims 1-32 in the manufacture of a medicament for the treatment or prevention of one or more symptoms associated with a skin condition or disorder.

54. A method of making a topical formulation comprising mixing one or more human milk fractions with one or more excipients, wherein the one or more human milk fractions comprise one or more of human milk cream or a sub-fraction thereof, human skim milk, human milk permeate, and human milk retentate, wherein the human milk permeate and the human milk retentate are obtained from ultra-filtration of human skim milk.

55. The method of claim 54, wherein the topical formulation comprises a sub-fraction of human milk cream, wherein the sub-fraction of human milk cream is human milk butteroil or human milk fat globule membrane solids.

56. The method of claim 54 or 55, wherein the human milk fractions are obtained from a pool of human milk collected from multiple human milk donors.

57. The method of claim 56, wherein the pool of human milk comprises human milk collected from at least 5, 10, 25, 50, or 100 human milk donors.

58. The method of any of claims 54-57, wherein the one or more human milk fractions comprises human milk cream, optionally wherein the human milk cream comprises from 30% to 70% human milk fat (weight/weight), optionally 55% to 65% human milk fat (weight/weight).

59. The method of claim 58, wherein the topical formulation is formulated with about or at least 1%, 3%, 5%, 10%, 20%, 30%, or 50% human milk cream (weight/weight).

60. The method of claim 58 or 59, wherein the topical formulation is formulated with less than 75%, 60%, or 50% human milk cream (weight/weight).

61. The method of any of claims 58-60, wherein the topical formulation is formulated with from 10% to 70%, from 15% to 55%, from 15% to 20%, or from 30% to 37% human milk cream (weight/weight).

62. The method of any of claims 58-61, wherein the human milk cream is mixed in an amount sufficient to provide a final concentration of from 1% to 40% human milk fat (weight/weight).

63. The method of any of claims 58-62, wherein the amount of human milk cream is sufficient to provide a final concentration of from 10% to 30% human milk fat (weight/weight).

64. The method of claim 62 or 63, wherein the amount of human milk cream is sufficient to provide a final concentration of about 20% human milk fat (weight/weight).

65. The method of claim 62 or 63, wherein the amount of human milk cream is sufficient to provide a final concentration of about 10% human milk fat (weight/weight).

66. The method of any of claims 54-65, wherein the one or more human milk fractions comprises human milk permeate obtained from filtration of human skim milk.

67. The method of 66, wherein the filtration is or comprises ultra-filtration.

68. The method of claim 66 or 67, wherein the permeate comprises at least 0.1%, 03%, 0.5% or 1.0% human milk oligosaccharides (weight/weight).

69. The method of any of claims 66-68, wherein prior to mixing the permeate with the one or more pharmaceutically acceptable excipients, the permeate undergoes one or more steps for concentrating human milk oligosaccharide content to at least 0.5%, 1%, 3%, 5%, or 10% human oligosaccharides (weight/weight).

70. The method of claim 69, wherein the one or more steps for concentrating human milk oligosaccharide content comprises one or both of nanofiltration or reverse osmosis.

71. The method of any of claims 66-70, wherein prior to mixing the permeate with the one or more pharmaceutically acceptable excipients, the permeate undergoes one or more steps to reduce the lactose content.

72. The method of claim 71, wherein the one or more steps to reduce lactose content comprises (i) contacting the permeate with a lactase enzyme to produce a permeate/lactase mixture, (ii) incubating the permeate/lactase mixture at a temperate of between 45° C. and 55° C., and (iii) filtering the permeate/lactase mixture to remove at least 90% of the lactase from the permeate, thereby reducing the lactose content of the permeate.

73. The method of any of claims 54-72, wherein the topical formulation is formulated with at least 5%, 10%, or 25% permeate (weight/weight) obtained from the ultra-filtration of human skim milk.

74. The method of any of claims 54-72, wherein the topical formulation is formulated with less than 90%, 80%, or 75% permeate (weight/weight) obtained from the ultra-filtration of human skim milk.

75. The method of any of claims 66-74, wherein the human milk permeate is mixed in an amount sufficient to provide a final concentration of at least 0.1% human milk oligosaccharides (weight/weight).

76. The method of claim 75, wherein the amount of the human milk permeate is sufficient to provide a final concentration of at least 0.3% human milk oligosaccharides (weight/weight).

77. The method of claim 75, wherein the amount of the human milk permeate is sufficient to provide a final concentration of from 0.1% and 0.5% human milk oligosaccharides (weight/weight).

78. The method of claim 75, wherein the amount of the human milk permeate is sufficient to provide a final concentration of from 0.3% human milk oligosaccharides (weight/weight).

79. The method of any of claims 54-78, wherein the topical formulation is formulated with from 10% to 70%, from 15% to 55%, from 15% to 20%, or from 30% to 37% human milk cream (weight/weight) and human milk permeate obtained from the ultra-filtration of human skim milk, wherein the human milk cream and the human milk permeate make up from 70% to 90% weight/weight of the topical formulation.

80. The method of any of claims 54-79, wherein the one or more excipients comprise one or more of a hydrocolloid, an emulsifier, a structuring agent, an antioxidant, a silicone containing compound, a thickening agent, a preservative, an additional skin conditioning agent, an emollient, a humectant, or an essential oil.

81. The method of any of claims 54-80, wherein the topical formulation is or comprises an oil-in water emulsion, a water-in-oil emulsion, a nanoemulsion, an emulsified gel, an oleaginous ointment, a hydrocolloid system, or a cream preparation.
